SEQUENCE LISTING (77 sequences)

SEQ ID NO: 1 - DNA, 401 bp, Homo sapiens
cccccatcctgggaagagcattttgtctggaaattctaccaggaaaacaaggaagggttgggcccctataatttctgcacctgctcaaacaggaagtagcagaaagtttggggccaagaaaaacaagttcaaggtcatcctggatatcagcatcccccatagccaaggagtcaccccatcccaggaggagctctcttggcrattaatgattaacagacagatggggtataaagaggggatggcccaggacccaccctttagcctgtcaccaaaggcttaagaggaagaaaaattcacctagtcttgtctagcatacctttggtgacagggagctcactaccaaaccagccccccattgccctgagaaagtatgaaagtgaagctgccttcccacaactggg

SEQ ID NO: 2 - DNA, 401 bp, Homo sapiens
tgggcctgtgggggaagaaatttcaagttggtcaagcctgttgtagccttggggttttagccttacgatggctctgtgagcttggcagggcaggccttgctctcagtttaggggagagaaaacccaagccttccaggagggctagggccttggtcagatcaccctgagctcatggcagagccaagtgtagactgagttcayggaccactggggcctggcctgccccaggctgggagatgcatcttttagggaaaaaagataggaaacccacttaagggttggccaggtgggcctgaccatggcaggccaggatgcccagtggaaccaggagccccattgtctagggccagctctgccggcttgctccatcaccctggggcagtcactacctccctttccct

SEQ ID NO: 3 - DNA, 401 bp, Homo sapiens
cctagccggcctcaggcacttgcggtgactcacaccccccacccctaagtctgaatgttgagagccccaggctaagaccacttgcctctgccactttccatgtgactgaggcaagacctccttgccaaggcctcagtttccccctctgtacttcaaaggaggcagctcaggcaggtgtcttaataatttaaacgacgaatrttgctaaagagataaggggcggggagagagagagatcactgtgggccatggttagggaagggtggaggagctgggtcctgaaagggtggactttggtcgggtggagaaaatagccaagaaggcagtccctgctggcagcacagcaaagagaagtgagaattagggggagcagaggtgaggctggaggggactttggttgg

SEQ ID NO: 4 - DNA, 401 bp, Homo sapiens
tggaggaagatcccctagccggcctcaggcacttgcggtgactcacaccccccacccctaagtctgaatgttgagagccccaggctaagaccacttgcctctgccactttccatgtgactgaggcaagacctccttgccaaggcctcagtttccccctctgtacttcaaaggaggcagctcaggcaggtgtcttaataatktaaacgacgaatattgctaaagagataaggggcggggagagagagagatcactgtgggccatggttagggaagggtggaggagctgggtcctgaaagggtggactttggtcgggtggagaaaatagccaagaaggcagtccctgctggcagcacagcaaagagaagtgagaattagggggagcagaggtgaggctggagg

SEQ ID NO: 5 - DNA, 401 bp, Homo sapiens
gagagatcactgtgggccatggttagggaagggtggaggagctgggtcctgaaagggtggactttggtcgggtggagaaaatagccaagaaggcagtccctgctggcagcacagcaaagagaagtgagaattagggggagcagaggtgaggctggaggggactttggttggggcaagattatttggggtccaaataacaasgatgtttagacgtattgccattgtgctctggggagccacggaaagctcttgagctgaggagtgacttggagaaagtttttcagccaatgcaatagagggagttggtacctttccttctgccaccctgagtttctggtgcctacggtagggtagggattgttctgccctgccttcagtgctttgagtgcttgaaacagaca

SEQ ID NO: 6 - DNA, 401 bp, Homo sapiens
acttctttcttccctttccaggaacttaacgggctccccacctcttgggtgctaggacaagtgggtggacctcagggaaggaaccgtgacccctcgagcgggtgggcgccagctcctttgcaaagtttctctccatcaagctcaggaggcaggacgagacggagggggaggtgcgagcagcattttctttttccagtaacrggatcctcggctggaaatgcgtgtgatcccgggggctgcgtttgggcgggtggtggccgggacttccttccctggctgagtaggagggagggaagcccctgggaggaggaggccttcaaaggggctctggggaggctggagtcctaggcagaggttcaggcccaccgagggtgcccgtggggtggaagggaggagaaaga

SEQ ID NO: 7 - DNA, 401 bp, Homo sapiens
cgagacggagggggaggtgcgagcagcattttctttttccagtaacaggatcctcggctggaaatgcgtgtgatcccgggggctgcgtttgggcgggtggtggccgggacttccttccctggctgagtaggagggagggaagcccctgggaggaggaggccttcaaaggggctctggggaggctggagtcctaggcagagrttcaggcccaccgagggtgcccgtggggtggaagggaggagaaagacggcgccttagcagggacctgggccgagttgacaccatcaggttatcttgaaagtgggaagccgggccctgtgccaacaaatgccatgcagggaatctggtagaccaagaggggccacattcccaccctgcaggctgccgttgtgtgacctggg

SEQ ID NO: 8 - DNA, 401 bp, Homo sapiens
cctggctgagtaggagggagggaagcccctgggaggaggaggccttcaaaggggctctggggaggctggagtcctaggcagaggttcaggcccaccgagggtgcccgtggggtggaagggaggagaaagacggcgccttagcagggacctgggccgagttgacaccatcaggttatcttgaaagtgggaagccgggccctrtgccaacaaatgccatgcagggaatctggtagaccaagaggggccacattcccaccctgcaggctgccgttgtgtgacctgggctgagctgcctctgaacttcagttttcttgtcggtgtgagggtaaatgaagcaggcaggtagtgttcggttgcttccaccagaatggggaagacttttcccacagtcaggtctgagt

SEQ ID NO: 9 - DNA, 401 bp, Homo sapiens
tgtggaggattcagtgaagaaactgagaaatgggagctttgaaaatgcccagtactgttcagaccaccctggggcctctccttctgcacccacctcacgtcacctcacccacccgcccctcctttgcctcagaaatgccctgtgtgaccttttcctcttctcacctcagcccagttccatccctgactgtaatgggagtckcgctcctgttcagctgacctcctgctcagctgaccaggcaggggataatcaatgtgtcaggggctgtcacctgagccccagctcccagaccccacatacacaggatcactgcttgtgtcccagcttgaaggagctcacggcctgcagagcggggaggagactgtctctgaacagaagttatatctggggagatcagggag

SEQ ID NO: 10 - DNA, 401 bp, Homo sapiens
agattccgtctcaaaaaaagaaaaaagaaaagaaaaagaaagaaaacgtgatgccagtggggactggacccacctaggtggtttctcggcatccctaccctgagaggctgccacaggtctcactggtacttgttcagagcagcagagctttagcgagccacctagtatgtgtgacctgggtgccaggggctgccacagcaratatagccagcccccgccctcgggagctcagaggccaaggggaagagtcttggcataaggttagcagatggtgtgactggaaaggtaggcagggccttgagtggtgagcaaagaggggagaacagcccctggcagaccactgggaatcaaaggaaggattttgaaaataacaggaatgataacagtgatctccatgtctt

SEQ ID NO: 11 - DNA, 401 bp, Homo sapiens
gctaatttattcttttgtttcattatcatttcccacttgggtctcgcatccccagggccctgaggtggctgttggaggtatttatggtcccgggtggggtgcagggaccagttagaaaccagtcccactcaggccatgatgcatgggtgtgttcacatgtgaggaggagccaggcacagactgagctcaaggagagggaaygcatgggtcagggccttgaaacattctcaggagtggtgctgagtggagagatgggggagggcagtgggctagcatgtttgtggtgggctctagagcttgggagcttcagagttgctgcagttgtggctggggttggagccccaggtgctgtgcagggaggttgggctcttttcctggggctgggggtagggggactgtca

SEQ ID NO: 12 - DNA, 401 bp, Homo sapiens
gcaaaaccttctcaatgaggtaagaaacaagacttggatgcaggtctctgccacatttcaatgcaaaaaggcaggaaataaactaattatggttattgggaaagagaggtgaaagatggaattgtcttcctagacttttccaacagaaaaatggccccttttaggtaaagtggcctgaatacacaataaatatacaaaatygagattgcttccccagataccagttggtgcctgttagtgagattgcagagggtgggggtgggctggtgatggctaccaggagaggtgggtggataggggaccgaaaatctggacaacccagtgagggtgcacacacagtctggatggtcactagacccctggccaaggtggaaagcctgtctccagtgcacaccccacaa

SEQ ID NO: 13 - DNA, 401 bp, Homo sapiens
taaaaaaagaaggcctggagtgatgaggagttagttacagctgcagccagggtgtagggtggaggctggaggcaccctgtcctcctcctccttggtgggaggcagctagggcagccattgccctccctccactaaagcctcagacgcctgactctaccccagagccacaaccaccacctgagggcagggccccacccaggragaaactcaggctgaggaagccaccctgtggcctttgagccagtcccttctgaccttgaacttcagcctccccatccacatgggggcttggtcccatggtgtggtagcatcggacctcagaggagtgggtggtgcctagcatctcagcctggatcagactcttgcccccacaccctttctggcctctggcactagcaggg

SEQ ID NO: 14 - DNA, 401 bp, Homo sapiens
gctttgagaaggccacctcccttcacagccaatgacttcactgggtgttcatggcaattgtctctcacagtcttggtcaaagcttgggagggaggcaggggagtggccccgttggcatctggttgcctcctccacctgcctgggatagctcaggtgggacctccccggcctgcccctgccccctggatcctgctgtcattyatgacctgccaaccatgccctttcccagggcatgcgacaggtctcagggcctcagccatgtttctccaccaaagcccttagtgctgagggttcagagtcccctggtgtgaggtccctggagcctggcagacacctgaggcctaatccccagagctctctaggtcctcgaaggaggccacaggccccagtgaatctgctgg

SEQ ID NO: 15 - DNA, 401 bp, Homo sapiens
gtgtttaaaaatggatatggtgaaattgcacatggcagagaggagcaaacacatatggacaccctgagacctccttagtttcctgaactccagcagcacagagtaaatgccagctctctgccagccctggaatgggccctgcagatgcagagaaaagaatcgtgggaagaaaactccagaagaaaaaatggggaaagtgayactgccgactcaaattactagaaatcctcagaaagtaaaaaaggaaaaagaaactcagggttgagagaagactttgggccggtgaattcatccttggagtgttcaggaaatccagagatttttggaaggctgggaattctttggaagtgaggcgtgtctggagataaggaataggaagcatgggctgtcaaaagcaattg

SEQ ID NO: 16 - DNA, 401 bp, Homo sapiens
ggaggttgcagcgagctgagatcgcgccactacactctagcctgggtggcagagtgaaattccatctaaaaaaaaaaaaaaaagaaactgaccttgactccaccccatacccatgggccttctgaggccaggactcccaggtgaaccttggcccgcagatggtaaatcacccaggagaaatagcctataaaatcctctccyaaaatggtgcattttgtattctgtagtgaaaagggcttttaaaagcactgcttactcaggtggatttatggagtttcccccacccctccctcccactagtgtttactggtcctcagtcctggagctccctggggtcctgttttttccttggtggctcttggtccagggagagaaacaagatagcatctctttcctgcctt

SEQ ID NO: 17 - DNA, 401 bp, Homo sapiens
agcatctcctcctctgggaagccttccctgacagggttaggcaaagttagcgactcccttttctgggctctcccagcatcccacatggaccctctcatagcatttctttcttacaagggactggagttctcatgagtcagctctccccattacactgtgtgccccctcagagcagggaccttgtcttatccataaccacaygcccatactagctcagggcttgtcttagtgggtgcccagggaatgtgttagagtccaaaggaagaaaaagctgaaatatcagaagttgtttctggagaaagggtttatctaggaggtggggcttgaatagcaccttagaaggcgttgaagttcaagaaacagcaagaatgagagagagcatgccaggtcagccgagacag

SEQ ID NO: 18 - DNA, 401 bp, Homo sapiens
cttgaacctgggaggtggtgattgcagtgagccgagatcgtgccactgcactgcagcctggcaacagagtgagactccgtctaaaaaaaacaaaaaacaaaacaaaacaaagaacaaaaattagccgggcatggtggtgcatgcctgtaatcccagctactcagaaggctgaggcaagagaattgcttgaacccaggaggygaaggttgcagtgagctgagattgcaccactgcattccagcctgggcgactgacctagactccatctcaaaacaacaacaacaacaaattcttatcattagattattctcacctcgataattcaggatgatttcccatctcaaaatccttaacctcaaccacatctgccaagttccttttgccatgtaaggcaacatatt

SEQ ID NO: 19 - DNA, 401 bp, Homo sapiens
ggtgcatgcctgtaatcccagctactcagaaggctgaggcaagagaattgcttgaacccaggaggcgaaggttgcagtgagctgagattgcaccactgcattccagcctgggcgactgacctagactccatctcaaaacaacaacaacaacaaattcttatcattagattattctcacctcgataattcaggatgatttcycatctcaaaatccttaacctcaaccacatctgccaagttccttttgccatgtaaggcaacatattcacaggttctgggaattaagagtgtggacttctttgggggaccatcattctgccttactcattctggatgtaacacagtttgtttaaccattcatctatagaaggatgtttggttgtttccagttttggggctat

SEQ ID NO: 20 - DNA, 401 bp, Homo sapiens
ttgagagtttttatcatgaaaggatgttgaatttattaaatgatttctcagcatcaattgaaatgattatatggtttttgtccttcattctgaagatacaatatattacattgatttacatatgttgaaccatccttatattcctgggattgtaaatgtagatttatttatgctctctgttattaggctgtggggctcaarttttattcaacctagtataattatcacctaacatgatgcttggcctgtggtaagctcaataaaggaaaggccatatttcttaaccacagagcaatgaaagtgggaattaataacaagagcttacctgctgcttagaaatctcatcaacagatgaatggataaggaaaatatggtgtgtgtatatatatatacacacacac

SEQ ID NO: 21 - DNA, 401 bp, Homo sapiens
attttggtatctgattggattttggtatcctggatccaatacccctgggataccgagagacgcctgtatggcactgcacaaagcagtgggctccctggaggaaaggcgcagggagctcagagagagtgttgcaggagggctgtggaggaggcggtgaattgagagaggggagtggtggtttttctggaaaggaagagtgartgcgagtcagccaggtggctcaagagagctgtggcagggaaggtgagccagaaagagtggccaaaggccagcagggccagggcagctagggcaggggctgtgtttcagatttcactctggctgttttatggagaaccacactgaggaggcagcggaggatttggggggacagttcggggggatggaggtccccaaaggta

SEQ ID NO: 22 - DNA, 401 bp, Homo sapiens
ttttttttgagacggagttttgcttttgttacccaggctggagtgcaatggcgcgatctcagctcaccgcaacctctgcctcccgggttcaagcgattctcctgcctcagcctcccgagtagctgggattacaggcatgcaccaccacacccggctaattttgtattttcagtagagacggggtttctccatgttggtcasgctggtctcgaactcccagcctcaggtgatacacccgcctcggcctcccaaagtgctgggattacaggcatgagccaccgtgcctggctgggaccctggctttgataaggtctgcaagaggcctctggggcaacagcttggacttcatgagttgggcgagcctgagaacagccctgaggtgggggggttttgtggttccc

SEQ ID NO: 23 - DNA, 401 bp, Homo sapiens
gtggaacccaccctaatccccagccccatgtgaccttgggtgagtcgctccctcccttggtctcacttccccatcttcaggaagggatgctgccccaccctgactgctctgtgctgggatgagagagccaaccagaccctcagtgattgcagagttgatgccagatcccagtttgccatgggttagggagggtcctctggrtaagcagctgattttgagggcctcagcttcccccgttcatcagtttctctcaaggttccttctacctcggacattccggggctttatgacaggctgtcgacgtgtacgagggagacgtgcatgtcctcttctctgggcttctggaggactggaggggacctgtaggtggccctttggggaatggggagatttttagatcc

SEQ ID NO: 24 - DNA, 401 bp, Homo sapiens
tccgccatacaaaggaatgaattaatggcattcccagaaacctggatgggaattggagaatattattctaagtgaagtaactcaggaaaggaaaaccaaacatcatatattctcactcataagtgggagctaagctatgaggatgcaaaggcagaaaaatgacacaatggactttggggactcagggaacgggtggaaagrgggtgaaggataaaagactacaaattgggtttagtgtatcccaagttactgctcgggatatgggtgcaccaaaatctcacaaatcaccactgaagaacttactcatgtaaccaaatgccaactgttccccaaaaacctatggaaataaaaaatttaaaaaataaaaaactgaggggatgcttgagctgggtcctgaaggg

SEQ ID NO: 25 - DNA, 401 bp, Homo sapiens
aaaagactacaaattgggtttagtgtatcccaagttactgctcgggatatgggtgcaccaaaatctcacaaatcaccactgaagaacttactcatgtaaccaaatgccaactgttccccaaaaacctatggaaataaaaaatttaaaaaataaaaaactgaggggatgcttgagctgggtcctgaagggtaagcaggaatkcaccaggtgggtgcagagggaagaccttggtggcagcggtccaggtcccaccaacagggcgaagactggtggtgggaaagagtgtctggaggatgaccaagggggctgaaatgggttggggtgcaagcgggaggtggtcgctttttgacacagcttggtctctgagagttcgccttgggaggcttttgaggggagggtaa

SEQ ID NO: 26 - DNA, 401 bp, Homo sapiens
gccttgaattggtcctgctgggttcaggaagtgaccaaatgaccatttttccttccttagccccctcatcttatcagctaccaccatgggcacaccctggcagggtcacagcagaccagctccctctgggcccagatgagggagggaccgaggacactgtgtttcagggcctgtagactccaggctggtcaggaaggaaaygcataggaacgtgagtgagagagtacgggtccactggccctgcctccaccacccacccgcttgctgtactatccaggaaggagtagcctgggagagcttgcgggaaccacttggggcctccctgggcctgccctgcccctattcctcttcctcccagagagcttgggctgcagggatggatgcagcctctgctttacatc

SEQ ID NO: 27 - DNA, 401 bp, Homo sapiens
ctccagcaatcctccttccagccttggccttccaaagtgctgggattacaagcatgagccaccatgtccaacccatgtttatttgaaatctatattattaaattagaacaacatggctatatgaaataacaggcttattttttctaacaaatataaacaggcataaggttttaagtgtgtacatactggcttttccatatmattttgaaactttctttacctttctgtatttgacaacttggaagtcaaattttaacttatctccaaagttctaatttaatgatttgatactaaagcaaaactgaatttcagtcctttgtgtaatttttagcctctagaacagttgactcagactgtatataagatcctgtaggggacatgaggttataatttgggaaagc

SEQ ID NO: 28 - DNA, 401 bp, Homo sapiens
gagtggggcaacttgcctgagccatcctacagaagggagcagtggccaagattctaatcctgcttcatccctgggttggggggactatccctgggagaggccagcactgtaccccaagaccctgagtgggtgtgaagagaggaagaaaggcccccacagagagcattccactcgccagttactgaacctggcagggactgrggtggtggcctttacaccttaacgggaaccagttcatctgattccatgctgcccaacaggcttaagtgcagggctggggaaatggaatttacagggatggccttctgatgtatttggaagtccccagggcaggaagaggagggggtacagaggctggcttgttcccttcacttccatcctcctcactcacactgcccaag

SEQ ID NO: 29 - DNA, 401 bp, Homo sapiens
aaacagattccagggccccactcccagcatccctgaatccagattttgatgggtagggccaaggaatctatattcttaaaagagctttctagtggctttcttgaggagccactggctaagatagttctggactcagcttgataacttttcaatctctgaatgtgccaaacgtggtcccacctcagggcgtttgcatctccrgttcctgctgcctcattcccacaaaccccagatcttcagatggacagctccttcctgtcattcaggcctcagctcaaatatcacaccctcagagaccttccctggaccccttatctcaagtgcctttgccccaatctctccatgatatcccattgccctgttttattttcttccacatagcgcttcctgaaactatctaa

SEQ ID NO: 30 - DNA, 401 bp, Homo sapiens
aagggaactgggatcgagttccaatggtctgtaagctcaggaggtggttcagaatcagaggaggttgcccactgctgctggggagtggagtctggtgggagagggacatggggacaactgtagagggctcagcatccagcccaccattcctgcccggaggatcctcaacatagagcactcgggggcagggcacagcagctsctcagaggggacgtgtttggcttggagctggtattccagaggtggggcagaggtgctaatgagacacatccttcccagttacatcccaggatgctgggtagggcccttccgcaaggacagtgcaagggtttgctcccttgttggtctctggttggccgcgttaatagtctggtgtagcctagtataaatcctacaaggtt

SEQ ID NO: 31 - DNA, 401 bp, Homo sapiens
accccccagtgatcccagagcttattggtgggtgctggattaaaggtgatttttattttcttctgttctgtaatagatatttactgctttttgtaatcttaggagttgggtttttttgtttttttttttcgagacagggtcttactctgttgcccagggcggagtgcagtggtgcgatcataggtcatcacaacctggaastccttggcccaggcaatcctccaccctcaaccttttaaacagttaacaccacaggcctgtgccaccacaacaaaaaattttaaaattttctgtagagatggggtctcactatttgcccaggctggtctcaagcttctggcctcaagtgatcctcccgccttggcctcccaaagtgttgggattacaggcataaaccatgg

SEQ ID NO: 32 - DNA, 401 bp, Homo sapiens
agatcacctgaggtcaggagttcgagaccagcctggtcaacatggtgaaacctcatctctactaaaatacaaaaattaggtgggcatggtggcacacacctgtaatcccagctactagggaggctgagacaggagaaccccttgaacctgggaagtgaaggttgcagtgagccaagatcgcaccatttgccctccagcctsggcgacagagcaagactccatctccaaaaaaaaaaaaaaaaaatgctgcattcatatccaagctgacccacttgccagctgtgtgacctagagcatgctactacatccctctgagcctgttgctgggtttacacagccaacattgactagagattatcacaagggttattataaagattcaatgagatcatagtggcaaa

SEQ ID NO: 33 - DNA, 401 bp, Homo sapiens
ataagtctggacaagaatcatggaacccaggtcctagccctagttctgccaccaatttgaagtcacttaatttcagtctctctaagccttagctgctcgtctttcaaaaagaataaaggcaggatgctaaggtagttgagtgcttgtggccatgtaaaaagatcacagatcagaaatacgagggtgctaaaaaagaatgayatcctaagtatgtgtttatatataatatatttacatgtatacatttatgacaaaatgaaaaaaattgaagtgataaacaggagaccttaataaaaatcctgtttctctctctctagaactgtgacctttggtcagtcacttcccttatctggacttattctcatatataaaatgaataggtttgattagatgatctgcag

SEQ ID NO: 34 - DNA, 401 bp, Homo sapiens
atacatataaatacacacaaatacacacaaatacacacagaaatacacacgcacacgcacacacacaaaacacatacacatacacacgaatacacacaaatacacacacaaatacacatacacgcacacacatgcacacacgcatacacacatacacacacatacacgtgtgcgcgcgcacacacacacacacacacgaasaacacagatagttgaggactccccagaccgctgaggaaagcttgcagtgtgaaaaggagaggccaacctaaccagcctgtaaaaggaaacagaggcaatgcagggaagagatgagccttaaaaaaataacccacaactggctggatgcagtggctcacgcctgtaatcccagcactttgggaggccgaggcgggtagatc

SEQ ID NO: 35 - DNA, 401 bp, Homo sapiens
gcccaggcccacacagcagcggggagccctcagcaggtgctgatctgcgctgccccctgctgggctatggagccctaggataaagtaccccttgccccaggcatcacccaggactcccagggctcctggggcgatgcacaggaaggacccctgagtggcatggcataggattccagtctcctaagcaacttgggccagcckaccccatgccctctgggcccctgttcccctgatgcaaggaaggggatgcccagaacttcagatcttggacttcgagacattccagtcccctgggatgtgggttaaagtgcagacacttgggcccaccgggcagagtctaagttagccagtctggggaacagcccaggagtctgcatttttcatcagcgggcagtggtccc

SEQ ID NO: 36 - DNA, 401 bp, Homo sapiens
aacgcggaattccaaggggtcatttcagacccaaaccttcagcaggtctgagcctcctactcaggggctaactgggcagggtcccaatccctctcccctcccttcctccaacagagagaccattggctgagacgtctcaatgtccactggagctctagtggccagttttacagcaggggagaccctgcttcttcccccacyagactcagtggtttcaaggctccacagccatgatctcaatctacctttccaattccccgagcaaggcaggaacccatcggaaccgtttcacggacgaagtggcttctctgaaatatagggaggttgccccagccctgcccactccacgggtgcttgctaggctcagccagcagagagttcaagggtatcctctgtgccag

SEQ ID NO: 37 - DNA, 401 bp, Homo sapiens
ttgtggggggtctgaaagagcctggaaatttctgaagtgccctaccaggtccgaggttatttttaggcattgaattagtgctgtccaatgaacagccccagggtctccttgaccccacacacggctctctttctccacctgctgacctcctgaggcattgaccggccacacggaggcctggcctctccagccccctccackccatagccaccctctgccaagcttccccttcgcacatagacatacacgcacatgcacacacacatacacacgcacgcgcacacacacacacatatacacaggtctgaggctgggtgggccgcactggcacacagggccccatggtatcaggcatcaaacctgcctggtgttggctgttctggctgagggggctgggggtg

SEQ ID NO: 38 - DNA, 401 bp, Homo sapiens
gcgttggtccctgttcccactctgcttagcccaaggtgttttcacggaaggtggggtccagagcagcccaggaaacggaaaagagacctcacttcaacgacccagccctgccaatcgcctccctgataatttaggattggccaggattgaacaaaaacacccaaaacttgggtgccccagggggtgcctggctctggataragaggtttggttggctgccctatcaccaattcccagggtgagctgaggaaggtctgaagtttgggagaccgctggaagccttggggatcggtaggctggcaccccccacccccaactcccgccgcccgcgggccccagctgggggcgccttcctcgcgatcccctgtattgtactctcccaagaacccagcgccagccct

SEQ ID NO: 39 - DNA, 401 bp, Homo sapiens
caaactcctgggcccaaacgatcctcctgcttcggcctcctaaagtgctgggattacaggtataagccaccatgctcatgcttggttttatttcttgaccaggataggagacacacagtgtttgttttattcttcatcactttttttttttaaggtgcagagagtcatggtgggaggccacataaaacccagtcctactcygaacttcagtttccgtattttaataacctggtcatcctttctgggaaatacctagcacaaagactggctcatgaggaagtgctcagtaaatcttaagaacatgaatagcccagtctgcaatcttcactggggtccttcacaccctagtcccagtcagctgagactgcccccgcccaagcaccaccatacaaccaggtctc

SEQ ID NO: 40 - DNA, 401 bp, Homo sapiens
tgtagattgttgcaccttgctatgggtctgtgtcctggagcagactgtgagctcttccaggtcgcaaaccttgcagccccaccacctgcaagaggcctggcctcaggaaggtttattgaatgaagaaaaggactgagtgacccaaaaactcagaaaccaaaagggatcattccaaactgtcaaaaggaaaaagcaaccccrccacctgctggactgactgcttggtcttcattgagtgttcttgatgggaatgtgaacttcaaagtcaaagagtgctcggggcctataatcccagcactttgggaggccgaggcgggtagatcacctgaggtcaggagttcgagaccagcctggtcaacatggtgaaacctcatctctactaaaatacaaaaattaggtgg

SEQ ID NO: 41 - DNA, 401 bp, Homo sapiens
atgccctctcctcttcccctgcagcctctgtcccgggaagctgtaaggaagtttcacaatgttctgggcctgctttctctgatgtcgcatttcttgtgtcctcctggtgctctctctctcattgtcgcccccctgacacaggctcccatgcacacacaggcccgtcttcccacccaggcccacacctacctctccggaacrgtgagctctttctagatccgcactacccagcccaggcccacacagcagcggggagccctcagcaggtgctgatctgcgctgccccctgctgggctatggagccctaggataaagtaccccttgccccaggcatcacccaggactcccagggctcctggggcgatgcacaggaaggacccctgagtggcatggcataggat

SEQ ID NO: 42 - DNA, 401 bp, Homo sapiens
acctgccaggctcacagcaaaacagggccaaatgccttccgcccccaccccaaacaagtcactgctcaggacttcttaattttcctccccacaggttgcagggcttctcattctgtgatttatagcccatcatataaattcccccattatctttcacatgcagcgctcggcctctctgccatttaataatacatttgactsgctgggcctctcccgggagaaatctaaagcttggctgcaattcgggggcaggcaacgcggaattccaaggggtcatttcagacccaaaccttcagcaggtctgagcctcctactcaggggctaactgggcagggtcccaatccctctcccctcccttcctccaacagagagaccattggctgagacgtctcaatgtccac

SEQ ID NO: 43 - DNA, 401 bp, Homo sapiens
agaaaggtgctcctcttccacactcccacgagggcctccccacttcctctttggcgctgacagtgaactccacccccactgcccacccattctcagacctgcgcgtcaccccttccaaactgcgcaccgaggctgcacctgcccaggcttgtgctggagtgaggctgctttctaggcacaaggggacacctggggccacaktggctcagggtggggctcctgtgtacactaagggtggccatttcaccctcctccaaagacctgcagggttgaaggactttgagcagattcagaaaaaaacaaaggcttgtgaaatgtcagaactaagagagtaaaggcggggtcaagtgtggccggccggggcctctgcctcctccatctctcctctttctggccctact

SEQ ID NO: 44 - DNA, 401 bp, Homo sapiens
gctgagtcgccaagtagcaagtctgcagggctgactgatgtgcggggttgtgagtcatccttcctaaagtgatctgggcgcccccagcattgccccagagctcaaaggatgacagttcccgcaggaggccattccagggaagagaattctagggagggggagagcataggtaaaggtgtggaggtgggacattcctgcatygtggcaggtacagcaaagagggttagcaagaacagaagccagagagtgtgtgaggtagaatggctttcggtgccaaggtgaggagtcccttctttctgagggcattgagggcattagaagcagtcgggcttagggtcctgactgtgcctggcactgcccatctcccacttctaccgctttccctagaaaggtggctgaac

SEQ ID NO: 45 - DNA, 401 bp, Homo sapiens
ccacaccagggtcctggggccctgagcccctgggagatcctgatggcagaggtgttggtgcccaccaaggtcctcctaggatgcccctctgtgaggggtcaggggcgggcagggaacctgctctctggaaaggcctcaattgataatctgaaaagcgcgctagaccttccaaataaaaaggtctccactgcagactcgagrggcgtgccataattacctgggaagggcaggtcagctgcattttttcaaatttgcattttacaaagtgtttaatggatatttatatgaaagagcagcctattgggtaaatgcgtgactttaaattaggctgaggccttccccgctctgctgtgctggctgagctcggccatcagtggggccttcggtccctccctccctta

SEQ ID NO: 46 - DNA, 401 bp, Homo sapiens
ctgggcatagcggctcatgccaccagtcacgtagtttgtggaattacaggaatagaacttgaagccagggtaggcacagtggctcacatctgtaatcccaacactttgtttgggaggctggggcgggaggatcgcttgagtccaggagtttgagaccagcctggccaacatagagagacccctctctctacaaataaaaawattaggccaggcgcggtggctcatgcctgtaatcccagcactttaggaggccgaggcaggtggatcacctgaggcggggagttcgagaccagtctgaccaacatggagaaaccctgtctctattaaaaaatacaaaataatacaaaattagctgggcatggtggcgcatgcctgtaatctcagctactcgggagactgag

SEQ ID NO: 47 - DNA, 401 bp, Homo sapiens
aggggcccaccccaactctgccccagccctagcccagccccgctcttcatacacaggaagatcatgacccaccgactctcacatcctgctgccaggagagtacacagccacagtctttgcagagagtgagtaggcaacatggatcaaaagttaaaaatccataaacgtccattttaaattcatagattctccttctacatrctatccttcagataaactctcacaagcgtgtaagaacagatgtacaaggatgtttatgcactccggtttgaagcagcatacaaaaaaaaaaaaagcaccaaaactgtaaacaacgtctgtgtccatcacagggggcctggttgaataaattatagtctctcctgaaaatggaatcctgtgtgaccgttctgcagcgcgag

SEQ ID NO: 48 - DNA, 401 bp, Homo sapiens
cagggtctccttgaccccacacacggctctctttctccacctgctgacctcctgaggcattgaccggccacacggaggcctggcctctccagccccctccactccatagccaccctctgccaagcttccccttcgcacatagacatacacgcacatgcacacacacatacacacgcacgcgcacacacacacacatatacrcaggtctgaggctgggtgggccgcactggcacacagggccccatggtatcaggcatcaaacctgcctggtgttggctgttctggctgagggggctgggggtgcctctgaggtgcaagtcagcagggagcagccgagcccacggggtgagggtgggagtggaagagcagcttcctcccagcctagccacaggtggtggctg

SEQ ID NO: 49 - DNA, 401 bp, Homo sapiens
ttttctcttgagatggtcaggtcctcctccgtgcctgtacacacccctcttgcatgcctctgtatcttgcccgcgtggtgggagaggatggtttcatacaactggttcctaaccaagagaacagttaggtaaggacctgtgtgccaccatggggggtgggtaacttctcaggggaccctggctttttttttttttgagacrgagttttgcttttgttacccaggctggagtgcaatggcgcgatctcagctcaccgcaacctctgcctcccgggttcaagcgattctcctgcctcagcctcccgagtagctgggattacaggcatgcaccaccacacccggctaattttgtattttcagtagagacggggtttctccatgttggtcaggctggtctcgaac

SEQ ID NO: 50 - DNA, 401 bp, Homo sapiens
ggggccatatccactgaccactatcctaggctaggactagagtcaaggctgtgtgggattgaatcctgtctctgccactaatcaactcttgtgcctgtaaaatgggagagtaacaggattcactgcttttggtcattagaaggacttgctgaattgacactgcctgacaaaggggaagcactgaattgggattaagtatgmactgagtggccttgggcaactctgccctctctgagctttgctttcttctagcaggtctctgcaaaccctctaggccaaagtcctctaatctccctgtgctagcagtgtgcagctcccgagggtggtcagagcacagcggcaagctggctgtattgcctcagatgggaccgtgtccctctctgccctgtaaggggatacca

SEQ ID NO: 51 - DNA, 401 bp, Homo sapiens
cagaaatgtctacatttctgcccgttgctatgaatgcaagtttgagtgctttgccagatgctctctcatttaattcttacgaccgcctctgaaaaggttctagtatctattcccttttgacagatgaggaaactgaggctcagattttaagcaacttgcacaagatcacacagcatgtagatgacagagcctgttctcaarccccatctgtttgattccagatcctgggttttgtaaccacaacacccttttcacagataggctggggtggtgggtccttacctgatgatggaagtgaaactcaaaactgcagtgactgaagcatcataggatgaagacccaggcccacaagaccctggagcagggatagtatgaatgacaggggaggagtgggaaggagg

SEQ ID NO: 52 - DNA, 401 bp, Homo sapiens
cagagctttagcgagccacctagtatgtgtgacctgggtgccaggggctgccacagcagatatagccagcccccgccctcgggagctcagaggccaaggggaagagtcttggcataaggttagcagatggtgtgactggaaaggtaggcagggccttgagtggtgagcaaagaggggagaacagcccctggcagaccactrggaatcaaaggaaggattttgaaaataacaggaatgataacagtgatctccatgtcttgagcacttacacagcaccaggggttcttctaagtcctttacatgcatcctgccttaaatcatcattctcccaggtggtgttatcagctccactttctagatgagaaaactgaggctcagactggttaagtgacttgctgagc

SEQ ID NO: 53 - DNA, 401 bp, Homo sapiens
tcagagccaatttaaagagccccatttttggcgtgcagttgggcccaggcgctgtcttcagagaggacgctggcttccccctggaattacaggcgtcctcccgccctcccgcctcgagaggcaggaatgcgaaggatcagctttcagcagagctccggctgccgttcggggagttcaccgtgcaggcccgtccccagcggycgaattaactggttctgtctgtacgactgggagagcagctctcgctaaaaatagcgccctgcccagctcgacctgcgagccgccgttggattctgagccgggacggctcaaggagccgggagccctgcacacccctcaccccgggcctggcccgggaccgaccacggaggcggaggccgcgggcaccgcgctggccccac

SEQ ID NO: 54 - DNA, 401 bp, Homo sapiens
tgaggtcaggcgttcgagaccagcctggccaacatggcgaaaccccgtctctactaaaaatacaaaaattagccaggcgtggtgacacacgcctgtaatcccagttactcaagaggctgaggcaggagaattgtttgaacctgggaggcggaggttgcagtgagctgagatcgagccattgcacttcagcctgggcttgcygagactccatctcaaaaaaaaataaaaaataaaaaaagagagagagagaaagagagaaaagaaaagaaaaagaaaagagccaggaggggaagctgcttgtcagtttatacgcaaggctttgatccactatgttatttgactcttccacaacctcacaaaatattcatatacccctttataccttaggaaatggaggcaca

SEQ ID NO: 55 - DNA, 401 bp, Homo sapiens
cccggctaattttttgtatttttagtggagacagggtttcactgtgttagccaggatggccttgatctcctgacctcgtgatccgcccacctcggcctcccaaagtactgggattacaggcgtgagccaccgcgcctggccaattttttaatttttagtagagatgaagtcttgctatgttgctcaggatggtcttgatckcctaagttcctgatgctcctgcctcagcctcccaaagtgctgggattgtaggcatgagccactgtacccagcctctgctcagttttttcacccagagtggtgaggaccctggaggggttggcagaaacctctccctcccctcaacaccttcttcctccatcctcctactgtggcctctggccctcccagccagaaggtgc

SEQ ID NO: 56 - DNA, 401 bp, Homo sapiens
gccactgcagtccagcctgcgccacagagtgagactctgtctcaaaaataaagagaaagagagagagcaaggaagaagggagggagggagggagggggagacagagggaaagagagaaagaaagagaaagaaagaaaagaaagaaagagaaagaaagagataagtaaagataggaattgtgattctccaatggttaagagygagctttggggctgggcacggtggctctcacctgtaatcccagcactttggaaggccgaggcaggtggatcacctgagatcaggagtttgaaaccagcctggccaacattgcaaaaccccatctctactaaaaataccaaaattagccaggcgtggtggcagacgcctgaaatcccagctactcaggaggctgaggcatg

SEQ ID NO: 57 - DNA, 401 bp, Homo sapiens
tttagccaacaaggcggggcagctgttgggggggatgggtggggggtcccaggtgggcatttagctgcccgcatagcacttcggcatcacctgccatccgtggatcccagattcctgtctctaagagccccaagagcctataggctctgtctaggccagtggttttcccgttgtgtccaaggagaagctcagaagttggcrtgtaggagacacttccttccttgaacccagcttggactagagcagctcaccagatccaatagaatacaaacattgacgtcaggcgtggtggctcatgcctgtaatcccagcactttgggaggctgaggtgggaggatcacaaggtcaagagtttgagaccagcctggccaacatggtgaaaccctgtctctactaaaaat

SEQ ID NO: 58 - DNA, 401 bp, Homo sapiens
gagcgctggaatggacccagggcatgctcctcatctcagcggttcaggttttcattcttctatctccatccttctatttaattctgtacttactaagacctgggggtacagggaggggcttggagcctatttgcccagctgctgaatggggaggttggagagatggatacttatggctccagtaccaggagccaactgttycccttgacaactggggaaactgaggcccacagagccaaggccacttgcccgtggttacctaaagatgttaacgagaaatccgggtctggaactcagatccctttgtatcctgtttcggtgttggtgtagtttgttgctttccctaagatgagcccagatagggaaactgaagtgcctgggctcctggttgggtcttctgc

SEQ ID NO: 59 - DNA, 401 bp, Homo sapiens
ggtaggtcacttgttcctggtcccacagccagcctgcctgcctgcctgcctgcctgcctgcctaccttccttctttccttcttccttcctctttctttcttttctttctttctttttctttctttcttttctttctttctttctgtccatttctttccctttctttctttctttctttctttctttctttctttctttctktctctctttctttcctttctttctttctctctctctctttctttctttctttctttcctttctctctctctctctctctctctctctctctctctctttctttctttctttctttctttctttctttctttctttctttctttctttctcactttctttttttttgacagggtcttcctctgttgcccaggctggagtag

SEQ ID NO: 60 - DNA, 401 bp, Homo sapiens
gcctgcctgcctgcctgcctgcctgcctaccttccttctttccttcttccttcctctttctttcttttctttctttctttttctttctttcttttctttctttctttctgtccatttctttccctttctttctttctttctttctttctttctttctttctttctttctctctttctttcctttctttctttctctctctytctttctttctttctttctttcctttctctctctctctctctctctctctctctctctctctttctttctttctttctttctttctttctttctttctttctttctttctttctcactttctttttttttgacagggtcttcctctgttgcccaggctggagtagagtgacacgatcatagctcactgtagcctcaacct

SEQ ID NO: 61 - DNA, 401 bp, Homo sapiens
gtaagcctgccatatggagccatccccttctttcattttttgcagagggaggatcagagaggcaccgtgacttattcaaggttgcacagccacacagtgacaaatctaagcctcaagcccagatctcccaccagagttctgcccttaccacttctcttcttcccctctggagaagctgcctgggcctcaccccaacttggwtctatctccctgtctattctccctgatctcacccctcacatagcctgctccccagtcctcactgtatcctctgctccagcctcaccatcctacccactgtctccagtacatgctttgttcatttccacctctggggcttttctcatgctgtctctccctccaagaatatacttttctgctcgctatgcagtcaactccta

SEQ ID NO: 62 - DNA, 401 bp, Homo sapiens
agccaggcatggtggcacacacgcctgtagtcctagctgctcgggaggctgaggcaggagggtcacatgagcccaggcattcgaggtcgcagtgagctataatcccaccactgcatttcagcctgggcaacagagcgagaccttgtctaaaaaagaagaagaagaggaggaggaagaagaggaagaagaggaagaggaagrggaaggggaagaggaaggaaacaaggaaggaaggagaaaggagggaaggaaggaaagagaaagaaggaaagaaagaaaagaaaggaaagaaaggctgggcgcagtggctcatgcctgtaatcccagcacttggggaggctgaggcaggtggatcacctgaggtcaggcgttcgagaccagcctggccaacatggcgaaac

SEQ ID NO: 63 - DNA, 401 bp, Homo sapiens
gaggtcaggcgttcgagaccagcctggccaacatggcgaaaccccgtctctactaaaaatacaaaaattagccaggcgtggtgacacacgcctgtaatcccagttactcaagaggctgaggcaggagaattgtttgaacctgggaggcggaggttgcagtgagctgagatcgagccattgcacttcagcctgggcttgctragactccatctcaaaaaaaaataaaaaataaaaaaagagagagagagaaagagagaaaagaaaagaaaaagaaaagagccaggaggggaagctgcttgtcagtttatacgcaaggctttgatccactatgttatttgactcttccacaacctcacaaaatattcatatacccctttataccttaggaaatggaggcacag

SEQ ID NO: 64 - DNA, 401 bp, Homo sapiens
aatggttcacagatgaccaggcaggcctgcttgagaagggagggggctccctgacctcacactgggaccatatgccaccccctctggtttcccagtgggtgcggagctgtctgggcccttactgaagggggaggaagaagcccagagacctggaacctcagccccaggcctgggccagggagaaaggaggggctgtgggcrtcactggggcccagggcccacctggagcttggcagcgtttctgggtcagggcagggagaggaagtacacagagagagtggctccaggcttggccatctgggctgaaatgccggctgctagggttggggtgaggaaggagggctggggctggggcctggagagtggattagtctcccctgggccacaggattggagccctc

SEQ ID NO: 65 - DNA, 401 bp, Homo sapiens
gaaatgggtgatctgtcttaagattggaggaaacactatgctttatgggcaatttaaggcttttcaaaggtaattttggcaatatgcttttttttctctcttttctcactgacttcaaccctgacaccgcagagggactcaagactgccttgggctagcctccctcccctgcatctggtgcccctgctgacccaccctggyctggcccatgatgtacctggactttcagcccagctctgatgctacccactgcttcttttccagcagggatgctgatggttttctgcctagaggggaattttttgggtccatccctccctggtcttcacaagtagcctcctgtgtccccattattcaaactgactagtggggtataccaggagccaggaggggggtgtctc

SEQ ID NO: 66 - DNA, 401 bp, Homo sapiens
gcctttaactgccctgttcctgggccaggaagaaggaaaaggagcctgggctctttctctcctagctcctgccctgcccccttcccactcagtacccggagagtacctgccccaggcccgtggagatgctcggctcagctggttggcgatgcccattcccatcccaagggtagaaatgcacaagcttcctccatttcactrgctgaccctccttcctccacaaggcagatggattgggggtccatacgttctccctataggtagggaaactaaggcacagagaaggacttgaaaagctgaagtggggagaccatgccagacctttggagcccagctgtgcttacagatgaggtcctgagttccaagttactgccccctctcccctccacacacactcctca

SEQ ID NO: 67 - DNA, 401 bp, Homo sapiens
aactgccctgttcctgggccaggaagaaggaaaaggagcctgggctctttctctcctagctcctgccctgcccccttcccactcagtacccggagagtacctgccccaggcccgtggagatgctcggctcagctggttggcgatgcccattcccatcccaagggtagaaatgcacaagcttcctccatttcactagctgaycctccttcctccacaaggcagatggattgggggtccatacgttctccctataggtagggaaactaaggcacagagaaggacttgaaaagctgaagtggggagaccatgccagacctttggagcccagctgtgcttacagatgaggtcctgagttccaagttactgccccctctcccctccacacacactcctcagacttc

SEQ ID NO: 68 - DNA, 401 bp, Homo sapiens
tgaagtggggagaccatgccagacctttggagcccagctgtgcttacagatgaggtcctgagttccaagttactgccccctctcccctccacacacactcctcagacttcctgttgggggcccaggctaagaccacttgcccacaccactgataacagtgagttgattgtgagcctcccgtcccctccagggagggctggwacctggctgatcagagggttcctgctttgggaaacaaaatgggccagatgaactcaggccctgggagcaggaccgttgcccccgtcctgagtaggacttcctgggagtccctcacccccagaacccgtggacctggctacggaggttagtcatgcctggaagtgccaagatgtggacaggtggtgctcagaaccccacct

SEQ ID NO: 69 - DNA, 401 bp, Homo sapiens
aagtggggagaccatgccagacctttggagcccagctgtgcttacagatgaggtcctgagttccaagttactgccccctctcccctccacacacactcctcagacttcctgttgggggcccaggctaagaccacttgcccacaccactgataacagtgagttgattgtgagcctcccgtcccctccagggagggctggaamctggctgatcagagggttcctgctttgggaaacaaaatgggccagatgaactcaggccctgggagcaggaccgttgcccccgtcctgagtaggacttcctgggagtccctcacccccagaacccgtggacctggctacggaggttagtcatgcctggaagtgccaagatgtggacaggtggtgctcagaaccccacctac

SEQ ID NO: 70 - DNA, 401 bp, Homo sapiens
gagggagtactaagcctttagtactccttggagggagtactaagcctttagtactccttggagggagtactaaagacctggggaaggctttatctcagtggttcaggttttggttcttctatctctgtccttctatttaattctgtacttgctaagacctggagaaaggcttagtactgttaattctgtacttactccctycccctggacttggtctgggagaccgggaatctgggctcaagctttgtgactttgggcaagtcatttcccctttctgagcttagttttcctggttgtaaaatggacctgccgtatgtggaggattcagtgaagaaactgagaaatgggagctttgaaaatgcccagtactgttcagaccaccctggggcctctccttctgc

SEQ ID NO: 71 - DNA, 401 bp, Homo sapiens
gtgtgaggtccctggagcctggcagacacctgaggcctaatccccagagctctctaggtcctcgaaggaggccacaggccccagtgaatctgctggaatgtcagcctcttgcccttgcctttgtccttcctgacccaccctcatcatctttaataatcaggaagagaacagctccctttcaccaagctgaatgtgtgcccsgccccatgccaagagctccccctgattacctcatgtctcatctcagttctccaatcctcctagcaggtaggggttattgttgtcactttcctttaccaggtgggaaacggagctctgggtgtgatatttcctctgcattttcctgtcggggtggtgaaataactggtttgaacccagtccactggactcgaaagctcatg

SEQ ID NO: 72 - DNA, 401 bp, Homo sapiens
ctgttcacccaaagtctgaggggttgcttgcaaatcagttagggctgatctagaaagaaaacgtttgggaggccgaggtgggcggatcacgaggtcaggagatcgagaccatcctggctaacacggtgaaaccccgtctctactaaaaatacaaaaaaattagccgggcgtggtggcaggctcctgtagtcccagctactygggaggctgaggcaggagaatggcatgaacccgggaggctgagcttgcagtgagccgagatcgcgccactgcactccagcctgggtgacagagcgagattccgtctcaaaaaaagaaaaaagaaaagaaaaagaaagaaaacgtgatgccagtggggactggacccacctaggtggtttctcggcatccctaccctgaga

SEQ ID NO: 73 - DNA, 401 bp, Homo sapiens
aaaagaaagaaaacgtgatgccagtggggactggacccacctaggtggtttctcggcatccctaccctgagaggctgccacaggtctcactggtacttgttcagagcagcagagctttagcgagccacctagtatgtgtgacctgggtgccaggggctgccacagcagatatagccagcccccgccctcgggagctcagargccaaggggaagagtcttggcataaggttagcagatggtgtgactggaaaggtaggcagggccttgagtggtgagcaaagaggggagaacagcccctggcagaccactgggaatcaaaggaaggattttgaaaataacaggaatgataacagtgatctccatgtcttgagcacttacacagcaccaggggttcttctaag

SEQ ID NO: 74 - DNA, 401 bp, Homo sapiens
aacaaacctgcacgttctgcacatgtatcccagaactaaaagtaaaattaaaaagaaaaagtgtgcagtgggagccaggagagctgccaatttatcaatgcattattttagcaaactctttatttggcttcatcccctatccccagccccaacacacacacacacacacacacacacacacacacacacacacacacacamcactcttaaatagacgtacacagaggcataagctggacagctcttcgtctggaaagttctaacaatctgaagattccatgacttgttgcatggtgtgttttgtttgttttcgagacagagtcttgctctgtcacccaggctggagtgcagtggtgccatcttggctcactgcaacctctgcctcccgggttcaagcaatt

SEQ ID NO: 75 - DNA, 401 bp, Homo sapiens
agatctttcgtgtacattggtaccatgcctgcgtatagttctctgccatttcatcatgtatgtagatttgagtaaccaccactgcaatcagcatccaggactattccatcaccacgaagatgtcgcttgtgctccccctttaacatcacatccacctgcctgccccaccaccatccctaacccctggcaaccactaatccrttttccatctctattgctttatccctttgagaatgccacgtacatggaatcatacactatgtgaccttttgatactggctttcctcgctcagcataatgcccaggagatccatccaagttgtcgtgtgtatcaatagtgtgtttcttttttagggctaagtagtattccatactgtgtgtatttgttttctatgactgct

SEQ ID NO: 76 - DNA, 401 bp, Homo sapiens
tttgagacagagtctcactctgctgcccaggctggagtgcagtggcacgatcttgggtcactgcaactttcgcctcccgggttcaagtgattctcccacctcagcctcccgagtagctaggactacaggcacacaacaccacacctggctaatttttgtattgtcagtagagacagggtttcaccatgttgaccaggctgrtttcgaactcctgacctcaggtgatcctcccgcctcggccttccaaagtgctgggatcacaggtgtgagccactgtgccaggcctttttcccccctttttaaaaaattgcgataaaatatgtaaaacataaaagttatcattttaactagttttaagtgtacaattcagtggcatgaagtccatttgtaatgttctgcaa

SEQ ID NO: 77 - DNA, 401 bp, Homo sapiens
cacacacaaatacagaaacacacacatacacaaatacacaaatacaaaaatacgcacaaatacacaatacacacacaaatacacataaatacacaaatacgcacacacaaatacatataaatacacacaaatacacacaaatacacacagaaatacacacgcacacgcacacacacaaaacacatacacatacacacgaayacacacaaatacacacacaaatacacatacacgcacacacatgcacacacgcatacacacatacacacacatacacgtgtgcgcgcgcacacacacacacacacacgaagaacacagatagttgaggactccccagaccgctgaggaaagcttgcagtgtgaaaaggagaggccaacctaaccagcctgtaaaaggaaac

(19) United States
(12) Patent Application Publication
(10) Pub. No.: US 2013/0224739 A1
(43) Pub. Date: Aug. 29, 2013

(54) GENETIC MARKERS FOR RISK MANAGEMENT OF VASCULAR DISEASE

(71) Applicants: Gudmar Thorleifsson, Reykjavik (IS); Solveig Gretarsdottir, Reykjavik (IS)

(72) Inventors: Gudmar Thorleifsson, Reykjavik (IS); Solveig Gretarsdottir, Reykjavik (IS)

(73) Assignee: deCODE Genetics ehf., Reykjavik (IS)

(21) Appl. No.: 13/806,045

(22) PCT Filed: Jun. 22, 2011
(86) PCT No.: PCT/IS11/50009

§ 371 Date: May 9, 2013

(30) Foreign Application Priority Data
Jun. 22, 2010 (IS) 8904

(51) Int. Cl. C12Q 1/68

(52) U.S. Cl. CPC C12Q 1/6883
USPC 435/6.11; 702/19

(57) ABSTRACT

Certain genetic markers have been found to be useful for risk management of vascular conditions, including abdominal aortic aneurysm, myocardial infarction, peripheral arterial disease and venous thromboembolism. The invention provides diagnostic applications using such markers, including methods of determining a susceptibility of vascular conditions.

As genetic polymorphisms conferring risk of common diseases are uncovered, genetic testing for such risk factors is becoming increasingly important for clinical medicine. Examples are apolipoprotein E testing to identify genetic carriers of the apoE4 polymorphism in dementia patients for the differential diagnosis of Alzheimer's disease, and of Factor V Leiden testing for predisposition to deep venous thrombosis. In the treatment of cancer, diagnosis of genetic variants in tumor cells is used for the selection of the most appropriate treatment regime for the individual patient. Genetic variation in estrogen receptor expression or heregulin type 2 (Her2) receptor tyrosine kinase expression is used to determine if anti-estrogenic drugs (tamoxifen) or anti-Her2 antibody (Herceptin) will be incorporated into breast cancer treatment plans.

Genetic testing services are now available for many of the common diseases, providing individuals with information about their disease risk based on the discovery that certain SNPs have been associated with risk of disease.

The present invention relates to genetic variants that have been found to be associated with risk of vascular diseases, including abdominal aortic aneurysm (AAA), myocardial infarction (MI), peripheral arterial disease (PAD), venous thromboembolism (VTE) and pulmonary embolism (PE). Various methods of risk assessment and management of these diseases are provided.

### SUMMARY OF THE INVENTION

The present inventors have discovered that certain variants on chromosome 9q33 are associated with vascular disease. The present invention relates to the utilization of such variants in the risk management of vascular disease.

In a first aspect, the present invention provides a method of determining a susceptibility to a condition selected from the group consisting of abdominal aortic aneurysm, myocardial infarction, peripheral arterial disease and venous thromboembolism, the method comprising analyzing nucleic acid sequence data from a human individual for at least one polymorphic marker selected from the group consisting of rs7025486, and markers in linkage disequilibrium therewith; wherein different alleles of the at least one polymorphic marker are associated with different susceptibilities to the condition in humans, and determining a susceptibility to the condition for the human individual. In one embodiment, linkage disequilibrium between markers is characterized by values of r² of greater than 0.2.

The invention also provides a method of determining a susceptibility to a condition selected from the group consisting of abdominal aortic aneurysm, myocardial infarction, peripheral arterial disease and venous thromboembolism, the method comprising analyzing nucleic acid sequence data from a human individual for at least one polymorphic marker within the human DAB2IP gene; wherein different alleles of the at least one polymorphic marker are associated with different susceptibilities to the condition in humans, and determining a susceptibility to the condition for the human individual.

Another aspect of the invention relates to a method of determining a susceptibility to a condition selected from the group consisting of abdominal aortic aneurysm, myocardial infarction, peripheral arterial disease and venous thromboembolism, in a human individual, the method comprising obtaining sequence data about a human individual identifying at least one allele of at least one polymorphic marker, wherein different alleles of the at least one polymorphic marker are associated with different susceptibilities to the condition in humans; and analyze the sequence data to determine a susceptibility to the condition from the sequence data; wherein the at least one polymorphic marker is selected from the group consisting of rs7025486, and markers in linkage disequilibrium therewith. In one embodiment, the analyzing is done by computer processor.

A further aspect of the invention relates to a method for determining a susceptibility to a condition selected from the group consisting of abdominal aortic aneurysm, myocardial infarction, peripheral arterial disease and venous thromboembolism, in a human individual, comprising determining the presence or absence of at least one allele of at least one polymorphic marker in a nucleic acid sample obtained from the individual, or in a genotype dataset from the individual, wherein the at least one polymorphic marker is selected from the group consisting of rs7025486, and markers in linkage disequilibrium therewith, and wherein determination of the presence of the at least one allele is indicative of a susceptibility to the condition.

Further provided is a method of identification of a marker for use in assessing susceptibility to a condition selected from the group consisting of abdominal aortic aneurysm, myocardial infarction, peripheral arterial disease and venous thromboembolism, in human individuals, the method comprising (a) identifying at least one polymorphic marker in linkage disequilibrium, as determined by values of r² of greater than 0.2, with rs7025486; (b) obtaining sequence information about the at least one polymorphic marker in a group of individuals diagnosed with the condition; and (c) obtaining sequence information about the at least one polymorphic marker in a group of control individuals; wherein determination of a significant difference in frequency of at least one allele in the at least one polymorphism in individuals diagnosed with the condition as compared with the frequency of the at least one allele in the control group is indicative of the at least one polymorphism being useful for assessing susceptibility to the condition.

Yet another aspect of the invention relates to a kit for assessing susceptibility to a condition selected from the group consisting of abdominal aortic aneurysm, myocardial infarction, peripheral arterial disease and venous thromboembolism, the kit comprising reagents for selectively detecting at least one allele of at least one polymorphic marker in the genome of the individual, wherein the polymorphic marker is selected from the group consisting of rs7025486, and markers in linkage disequilibrium therewith, and a collection of data comprising correlation data between the at least one polymorphism and susceptibility to the condition.

The invention also provides use of an oligonucleotide probe in the manufacture of a diagnostic reagent for diagnosing and/or assessing a susceptibility to a condition selected from the group consisting of abdominal aortic aneurysm, myocardial infarction, peripheral arterial disease and venous thromboembolism, wherein the probe is capable of hybridizing to a segment of a nucleic acid whose nucleotide sequence is given by any one of SEQ ID NO:1-77, and wherein the segment is 15-400 nucleotides in length.

Computer-implemented aspects are also provided. One such aspect relates to a computer-readable medium having computer executable instructions for determining susceptibility to a condition selected from the group consisting of abdominal aortic aneurysm, myocardial infarction, peripheral arterial disease and venous thromboembolism, the computer readable medium comprising (a) data identifying the presence or absence of at least one allele of at least one polymorphic marker for at least one human subject; and (b) a routine stored on the computer readable medium and adapted to be executed by a processor to determine risk of developing the condition for the at least one polymorphic marker; wherein the at least one polymorphic marker is selected from the group consisting of rs7025486, and markers in linkage disequilibrium therewith, as characterized by values of r² of greater than 0.2.

The invention also provides an apparatus for determining a genetic indicator for a condition selected from the group consisting of abdominal aortic aneurysm, myocardial infarction, peripheral arterial disease and venous thromboembolism, in a human individual, comprising a processor; and a computer readable memory; wherein the computer readable memory has computer executable instructions adapted to be executed on the processor to analyze marker information for at least one human individual with respect to at least one polymorphic marker selected from the group consisting of rs7025486, and markers in linkage disequilibrium therewith, as characterized by values of r² of greater than 0.2, and generate an output based on the marker information, wherein the output comprises a measure of susceptibility of the at least one marker or haplotype as a genetic indicator of the condition for the human individual.

It should be understood that all combinations of features described herein are contemplated, even if the combination of feature is not specifically found in the same sentence or paragraph herein. This includes in particular the use of all markers disclosed herein, alone or in combination, for analysis individually or in haplotypes, in all aspects of the invention as described herein.

### BRIEF DESCRIPTION OF THE DRAWINGS

The foregoing and other objects, features and advantages of the invention will be apparent from the following more particular description of preferred embodiments of the invention.

FIG. 1 provides a diagram illustrating a computer-implemented system utilizing risk variants as described herein.

FIG. 2 shows RNA expression of DAB2IP in various human tissues. Shown is the average expression of duplicate experiments with the highest expression set to unity (1.0) for presentation purposes.

### DETAILED DESCRIPTION

### Definitions

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometime referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including SNPs, mini- or microsatellites, translocations and copy number variations (insertions, deletions, duplications). Polymorphic markers can be of any measurable frequency in the population. For mapping of disease genes, polymorphic markers with population frequency higher than 5-10% are in general most useful. However, polymorphic markers may also have lower population frequencies, such as 1-5% frequency, or even lower frequency, in particular copy number variations (CNVs). The term shall, in the present context, be taken to include polymorphic markers with any population frequency.

An “allele” refers to the nucleotide sequence of a given locus (position) on a chromosome. A polymorphic marker allele thus refers to the composition (i.e., sequence) of the marker on a chromosome. Genomic DNA from an individual contains two alleles (e.g., allele-specific sequences) for any given polymorphic marker, representative of each copy of the marker on each chromosome. Sequence codes for nucleotides used herein are: A=1, C=2, G=3, T=4. For microsatellite alleles, the CEPH sample (Centre d'Etudes du Polymorphisme Humain, genomics repository, CEPH sample 1347-02) is used as a reference, the shorter allele of each microsatellite in this sample is set as 0 and all other alleles in other samples are numbered in relation to this reference. Thus, e.g., allele 1 is 1 bp longer than the shorter allele in the CEPH sample, allele 2 is 2 bp longer than the shorter allele in the CEPH sample, allele 3 is 3 bp longer than the lower allele in the CEPH sample, etc., and allele −1 is 1 bp shorter than the shorter allele in the CEPH sample, allele −2 is 2 bp shorter than the shorter allele in the CEPH sample, etc.

Sequence conucleotide ambiguity as described herein is as proposed by IUPAC-IUB. These codes are compatible with the codes used by the EMBL, GenBank, and PIR databases.

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C, G or T |
| D | A, G or T |
| H | A, C or T |
| V | A, C or G |
| N | A, C, G or T (Any base) |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. A “marker” or a “polymorphic marker”, as defined herein, is a variant. Alleles that differ from the reference are referred to as “variant” alleles.

A “microsatellite” is a polymorphic marker that has multiple small repeats of bases that are 2-8 nucleotides in length (such as CA repeats) at a particular site, in which the number of repeat lengths varies in the general population. An “indel” is a common form of polymorphism comprising a small insertion or deletion that is typically only a few nucleotides long.

A “haplotype,” as described herein, refers to a segment of genomic DNA that is characterized by a specific combination of alleles arranged along the segment. For diploid organisms such as humans, a haplotype comprises one member of the pair of alleles for each polymorphic marker or locus along the segment. In a certain embodiment, the haplotype can comprise two or more alleles, three or more alleles, four or more alleles, or five or more alleles.

Allelic identities are described herein in the context of the marker name and the particular allele of the marker, e.g., “1 rs7025486” refers to the 4 allele of marker rs7025486, and is equivalent to “rs7025486 allele 1”. Furthermore, allelic codes are as for individual markers, i.e. 1=A, 2=C, 3=G and 4=T.

The term “susceptibility”, as described herein, refers to the proneness of an individual towards the development of a certain state (e.g., a certain trait, phenotype or disease), or towards being less able to resist a particular state than the average individual. The term encompasses both increased susceptibility and decreased susceptibility. Thus, particular alleles at polymorphic markers and/or haplotypes of the invention as described herein may be characteristic of increased susceptibility (i.e., increased risk) of a vascular condition, as characterized by a relative risk (RR) or odds ratio (OR) of greater than one for the particular allele or haplotype. Alternatively, the markers and/or haplotypes of the invention are characteristic of decreased susceptibility (i.e., decreased risk) of the condition, as characterized by a relative risk of less than one.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or they can contain information about multiple markers, and they may also comprise other factors, such as particulars about diseases diagnoses, racial information, biomarkers, biochemical measurements, therapeutic methods or drugs, etc.

A “computer-readable medium”, is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

A “nucleic acid sample” as described herein, refers to a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including a blood sample, sample of amniotic fluid, sample of cerebrospinal fluid, or tissue sample from skin, muscle, buccal or conjunctival mucosa, placenta, gastrointestinal tract or other organs.

The term “therapeutic agent” refers to an agent that can be used to ameliorate or prevent symptoms associated with a particular disease or condition. For example, the therapeutic agent may be an agent for treating or preventing a condition selected from the group consisting of abdominal aortic aneurysm, myocardial infarction, peripheral arterial disease and venous thromboembolism.

The term “disease-associated nucleic acid”, as described herein, refers to a nucleic acid that has been found to be associated to a disease. This includes, but is not limited to, the markers and haplotypes described herein and markers and haplotypes in strong linkage disequilibrium (LD) therewith. In one embodiment, a disease-associated nucleic acid refers to a nucleic acid associated with risk of a condition selected from the group consisting of abdominal aortic aneurysm, myocardial infarction, peripheral arterial disease and venous thromboembolism.

The term “antisense agent” or “antisense oligonucleotide” refers, as described herein, to molecules, or compositions comprising molecules, which include a sequence of purine an pyrimidine heterocyclic bases, supported by a backbone, which are effective to hydrogen bond to a corresponding contiguous bases in a target nucleic acid sequence. The backbone is composed of subunit backbone moieties supporting the purine and pyrimidine heterocyclic bases at positions which allow such hydrogen bonding. These backbone moieties are cyclic moieties of 5 to 7 atoms in size, linked together by phosphorous-containing linkage units of one to three atoms in length. In certain preferred embodiments, the antisense agent comprises an oligonucleotide molecule.

The term “VTE”, as described herein, refers to venous tromboembolism. A venous thrombosis is a blood clot that forms within a vein. If a piece of a blood clot breaks off it can be transported in the vein and is then called an embolism.

The term “PE”, as described herein, refers to pulmonary embolism. An embolism that lodges into the lungs is a pulmonary embolism. Pulmonary embolism is thus a subtype of venous thromboembolism.

The term “PAD”, as described herein, refers to peripheral arterial disease. The disease is also called peripheral vascular disease (PVD) and peripheral arterial occlusive disease (PAOD), and includes all diseases caused by the obstruction of arteries in the arms and legs.

The term “MI”, as described herein, refers to myocardial infarction, also commonly called heart attack. During an MI event, interruption of blood supply to a part of the heart occurs, leading to death of heart cells. This is most commonly caused by occlusion of coronary arteries following rupture of atherosclerotic plaques.

The term “AAA”, as described herein, refers to abdominal aortic aneurysm. This is a localized dilation of the abdominal aorta.

The term “LD Block C09”, as described herein, refers to the Linkage Disequilibrium (LD) block on Chromosome 9 between markers rs878708 and rs10985475, corresponding to position 123,385,920-123,785,952 of NCBI (National Center for Biotechnology Information) Build 36.

The present inventors have discovered that genetic variants on chromosome 9q33 are associated with risk of vascular conditions in humans. Through an association analysis of polymorphic markers in individuals with conditions such as abdominal aortic aneurysm, myocardial infarction, peripheral arterial disease and pulmonary embolism, certain marker alleles were found to be predictive of increased risk of these conditions. Without intending to be bound by theory, it is believed that such markers are useful in diagnostic and prognostic methods of conditions such as abdominal aortic aneurysm, myocardial infarction, peripheral arterial disease and venous thromboembolism.

### Methods of Determining Susceptibility to Vascular Conditions

Accordingly, the present invention provides methods of determining a susceptibility to vascular conditions in human individuals. In one aspect, the method comprises determining a susceptibility to a condition selected from the group consisting of abdominal aortic aneurysm, myocardial infarction, peripheral arterial disease and venous thromboembolism, the method comprising analyzing nucleic acid sequence data from a human individual for at least one polymorphic marker selected from the group consisting of rs7025486, and markers in linkage disequilibrium therewith, as characterized by values of r² of greater than 0.2; wherein different alleles of the at least one polymorphic marker are associated with different susceptibilities to the condition in humans, and determining a susceptibility to the condition for the human individual.

In one embodiment, markers in linkage disequilibrium with rs7025486 are selected from the group consisting of rs584985, rs2777310, rs2797348, rs12352132, rs2777308, rs2797347, rs1003016, rs12553641, rs12554667, rs10760182, rs10818577, rs10818578, rs10818579, rs10818580, rs10985344, rs885150, rs10818583, rs7025486, rs10985349, rs10818589, rs10116069, rs2416834, rs878708, rs669128, rs2009828, rs2777320, rs4836858, rs7038469, rs2777319, rs7869336, rs2797349, rs2777311, rs62575880, s.123444035, s.123444070, rs1768732, s.123445547, s.123446322, rs7465724, s.123446681, s.123447265, s.123449587, rs12554639, s.123451318, s.123451324, s.123451615, s.123451617, rs10818576, s.123455512, rs62572789, rs12380555, rs10985347, s.123459543, rs885150, s.123461786, s.123462115, rs1984038, rs1984037, s.123469017, s.123472214, rs12000685, rs12000723, rs35661033, s.123479977, rs1571804, s.123591409, and rs10985475.

In certain embodiments, determination of the presence of at least one copy of at least one allele selected from the group consisting of rs7025486 allele A, rs584985 allele C, rs2777310 allele A, rs2797348 allele G, rs12352132 allele G, rs2777308 allele A, rs2797347 allele C, rs1003016 allele C, rs12553641 allele A, rs12554667 allele G, rs10760182 allele G, rs10818577 allele C, rs10818578 allele G, rs10818579 allele A, rs10818580 allele A, rs10985344 allele A, rs885150 allele C, rs10818583 allele A, rs7025486 allele A, rs10985349 allele T, rs10818589 allele T, rs10116069 allele T, rs2416834 allele C, rs878708 allele A, rs669128 allele C, rs2009828 allele G, rs2777320 allele A, rs4836858 allele T, rs7038469 allele T, rs2777319 allele T, rs7869336 allele T, rs2797349 allele A, rs2777311 allele C, rs62575880 allele A, s.123444035 allele G, s.123444070 allele T, rs1768732 allele C, s.123445547 allele A, s.123446322 allele A, rs7465724 allele C, s.123446681 allele A, s.123447265 allele A, s.123449587 allele T, rs12554639 allele A, s.123451318 allele G, s.123451324 allele T, s.123451615 allele T, s.123451617 allele A, rs10818576 allele G, s.123455512 allele C, rs62572789 allele A, rs12380555 allele C, rs10985347 allele T, s.123459543 allele C, rs885150 allele C, s.123461786 allele C, s.123462115 allele G, rs1984038 allele C, rs1984037 allele C, s.123469017 allele C, s.123472214 allele A, rs12000685 allele C, rs12000723 allele C, rs35661033 allele C, s.123479977 allele A, rs1571804 allele T, s.123591409 allele C, and rs10985475 allele T, (“risk allele”) is indicative of increased risk of the condition for the human individual. Determination of the absence of at least one of the at-risk alleles recited above is indicative of a decreased risk of the condition for the human individual. As a consequence, in certain embodiments, the analyzing comprises determining the presence or absence of at least one at-risk allele of the polymorphic marker for the condition. In one preferred embodiment, the determination of the presence of rs7025486 allele A is indicative of increased risk of the condition for the individual. Individuals who are homozygous for at-risk alleles are at particularly high risk. Thus, in certain embodiments determination of the presence of two alleles of one or more of the above-recited risk alleles is indicative of particularly high risk (susceptibility) of the condition.

Alternatively, the allele that is detected can be the allele of the complementary strand of DNA, such that the nucleic acid sequence data includes the identification of at least one allele which is complementary to any of the alleles of the polymorphic markers referenced above.

In certain embodiments, the nucleic acid sequence data is obtained from a biological sample containing nucleic acid from the human individual. The nucleic acids sequence may suitably be obtained using a method that comprises at least one procedure selected from (i) amplification of nucleic acid from the biological sample; (ii) hybridization assay using a nucleic acid probe and nucleic acid from the biological sample; and (iii) hybridization assay using a nucleic acid probe and nucleic acid obtained by amplification of the biological sample. The nucleic acid sequence data may also be obtained from a preexisting record. For example, the preexisting record may comprise a genotype dataset for at least one polymorphic marker. In certain embodiments, the determining comprises comparing the sequence data to a database containing correlation data between the at least one polymorphic marker and susceptibility to the condition.

It is contemplated that in certain embodiments of the invention, it may be convenient to prepare a report of results of risk assessment. Thus, certain embodiments of the methods of the invention comprise a further step of preparing a report containing results from the determination, wherein said report is written in a computer readable medium, printed on paper, or displayed on a visual display. In certain embodiments, it may be convenient to report results of susceptibility to at least one entity selected from the group consisting of the individual, a guardian of the individual, a genetic service provider, a physician, a medical organization, and a medical insurer.

Markers in the DAB2IP Gene Associate with Vascular Conditions

The sequence variant rs7025486[A] maps within intron 1 of the DAB2IP gene (DAB2 interacting protein) also called AlP1 (ASK1-interacting protein). The DAB2IP gene is a member of the RAS-GTPase-activating protein family (Iwashita, S. & Song, S. Y., Mol Biosyst 4:213-22 (2008)). The DAB2IP protein has been shown to suppress cell survival and proliferation through suppression of the PI3K-Act and RAS pathways and to induce apoptosis through activation of ASK1, a member of the JNK and p38 MAPK pathways (Xie, D., et al. Proc Natl Acad Sci USA 107:2485-90 (2009)). The sequence variant rs7025486[A] also showed nominally significant correlation with the level of DAB2IP expression in adipose tissue, mammary artery and ascending aorta. Many studies have underscored the pivotal role of the PI3K-Akt signaling pathway in the vascular endothelium, affecting endothelial cell proliferation and survival as well as endothelial cell migration, and NO production (Dimmeler, S., et al. Nature 399:601-5 (1999); Shiojima, I & Walsh, K. Circ Res 90:1243-50 (2002)).

It this contemplated that other markers in the DAB2IP gene are associated with risk of vascular conditions such as abdominal aortic aneurysm, myocardial infarction, peripheral arterial disease and venous thromboembolism. As a consequence, in one aspect, the present invention provides a method of determining a susceptibility to a condition selected from the group consisting of abdominal aortic aneurysm, myocardial infarction, peripheral arterial disease and venous thromboembolism, the method comprising analyzing nucleic acid sequence data from a human individual for at least one polymorphic marker within the human DAB2IP gene, wherein different alleles of the at least one polymorphic marker are associated with different susceptibilities to the condition in humans, and determining a susceptibility to the condition for the human individual. In one embodiment, the at least one markers is selected from the group consisting of rs7025486, and markers in linkage disequilibrium therewith. In one specific embodiment, the at least one polymorphic marker is selected from the group consisting of rs7025486, and markers in linkage disequilibrium therewith, as characterized by values of r² of greater than 0.2.

### Vascular Phenotypes Affected by Variants on Chr9q33

The surprising discovery made by the current inventors demonstrates an association between certain sequence variants and a number of vascular disorders. The vascular disorders include Arterial and Venous diseases. These disorders have traditionally been considered two distinct entities, a belief, however, challenged by a growing body of evidence (Prandoni, P., J Intern Med 262:341-50 (2007); Sorensen, H. T., et al. Lancet 370:1773-9 (2007); Braekkan, S. K., et al. J Thromb Haemost 6:1851-7 (2008)) including the current finding. For example, large population studies have shown increased incidence of cardiovascular events (MI or stroke) in patients with VTE (Sorensen, H. T., et al. Lancet 370:1773-9 (2007)), and subjects with family history of MI have increased risk of VTE (Braekkan, S. K., et al. J Thromb Haemost 6:1851-7 (2008)). Although the pathophysiology connecting these disorders has not been clarified, it has been suggested that atherosclerosis may induce a prothrombotic state that promotes development of VTE (Prandoni, P., et al. N Engl J Med 348:1435-41 (2003)). However, factors other than atherosclerosis may contribute as studies have failed to confirm subclinical atherosclerosis as an independent risk factor for VTE and the evidence that arterial and venous disease share atherosclerotic risk factors has been inconsistent (Reich, L. M., et al. J Thromb Haemost 4:1909-13 (2006); van der Hagen, P. B., et al. J Thromb Haemost 4:1903-8 (2006)). Interestingly it was recently shown, in a large study of apparently healthy individuals, that treatment with statins (HMG-CoA reductase inhibitors), drugs that are effective and widely recommended for prevention and treatment of cardiovascular disease, leads to a significant reduction in the occurrence of symptomatic VTE (Glynn, R. J., et al. N Engl J Med 360:1851-61 (2009)).

The current discovery connects diseases of the vascular system that have traditionally not been considered strongly related. The findings suggest a pathophysiology shared by different vascular diseases that is affected by rs7025486 [A]. This may involve abnormal vascular remodeling response to endothelial disruption or inflammation, increased susceptibility to thrombosis, or even common biological triggers of vascular damage. Along the same lines, statins, beneficial to both atherosclerotic arterial disease and VTE, are thought to favorably influence thrombosis, inflammation and endothelial function in addition to their well-defined effect on lipid levels. Furthermore, the DAB2IP protein is a negative regulator of the PI3K/Act pathway that participates in numerous cellular functions for cell types such as vascular endothelial cells, endothelial progenitor cells, platelets and inflammatory cells that are involved in vascular remodeling (Morello, F., et al. Cardiovasc Res 82:261-71 (2009)).

### Obtaining Nucleic Acid Sequence Data

Sequence data can be nucleic acid sequence data, which may be obtained by means known in the art. For example, nucleic acid sequence data may be obtained through direct analysis of the sequence of the polymorphic position (allele) of a polymorphic marker. Suitable methods, some of which are described herein, include, for instance, whole genome sequencing methods, whole genome analysis using SNP chips (e.g., Infinium HD BeadChip), cloning for polymorphisms, non-radioactive PCR-single strand conformation polymorphism analysis, denaturing high pressure liquid chromatography (DHPLC), DNA hybridization, computational analysis, single-stranded conformational polymorphism (SSCP), restriction fragment length polymorphism (RFLP), automated fluorescent sequencing; clamped denaturing gel electrophoresis (CDGE); denaturing gradient gel electrophoresis (DGGE), mobility shift analysis, restriction enzyme analysis; heteroduplex analysis, chemical mismatch cleavage (CMC), RNase protection assays, use of polypeptides that recognize nucleotide mismatches, such as E. coli mutS protein, allele-specific PCR, and direct manual and automated sequencing. These and other methods are described in the art (see, for instance, Li et al., Nucleic Acids Research, 28(2): el (i-v) (2000); Liu et al., Biochem Cell Bio 80:17-22 (2000); and Burczak et al., Polymorphism Detection and Analysis, Eaton Publishing, 2000; Sheffield et al., Proc. Natl. Acad. Sci. USA, 86:232-236 (1989); Orita et al., Proc. Natl. Acad. Sci. USA, 86:2766-2770 (1989); Flavell et al., Cell, 15:25-41 (1978); Geever et al., Proc. Natl. Acad. Sci. USA, 78:5081-5085 (1981); Cotton et al., Proc. Natl. Acad. Sci. USA, 85:4397-4401 (1985); Myers et al., Science 230:1242-1246 (1985); Church and Gilbert, Proc. Natl. Acad. Sci. USA, 81:1991-1995 (1988); Sanger et al., Proc. Natl. Acad. Sci. USA, 74:5463-5467 (1977); and Beavis et al., U.S. Pat. No. 5,288,644).

Recent technological advances have resulted in technologies that allow massive parallel sequencing to be performed in relatively condensed format. These technologies share sequencing-by-synthesis principle for generating sequence information, with different technological solutions implemented for extending, tagging and detecting sequences. Exemplary technologies include 454 pyrosequencing technology (Nyren, P. et al. Anal Biochem 208:171-75 (1993); http://www.454.com), Illumina Solexa sequencing technology (Bentley, D. R. Curr Opin Genet Dev 16:545-52 (2006); http://www.illumina.com), and the SOLiD technology developed by Applied Biosystems (ABI) (http://www.appliedbiosystems.com; see also Strausberg, R. L., et al. Drug Disc Today 13:569-77 (2008)). Other sequencing technologies include those developed by Pacific Biosciences (http://www.pacificbiosciences.com), Complete Genomics (http://www.completegenomics.com), Intelligen Bio-Systems (http://www.intelligentbiosystems.com), Genome Corp (http://www.genomecorp.com), ION Torrent Systems (http://www.iontorrent.com) and Helicos Biosciences (http://www.helicosbio.som). It is contemplated that sequence data useful for performing the present invention may be obtained by any such sequencing method, or other sequencing methods that are developed or made available. Thus, any sequence method that provides the allelic identity at particular polymorphic sites (e.g., the absence or presence of particular alleles at particular polymorphic sites) is useful in the methods described and claimed herein.

Alternatively, hybridization methods may be used (see Current Protocols in Molecular Biology, Ausubel et al., eds., John Wiley & Sons, including all supplements). For example, a biological sample of genomic DNA, RNA, or cDNA (a “test sample”) may be obtained from a test subject or individual suspected of having, being susceptible to, experiencing symptoms associated with, or predisposed for a condition such as abdominal aortic aneurysm, myocardial infarction, peripheral arterial disease and venous thromboembolism (the “test subject”). The subject can be an adult, child, or fetus. The DNA, RNA, or cDNA sample is then examined. The presence of a specific marker allele can be indicated by sequence-specific hybridization of a nucleic acid probe specific for the particular allele. The presence of more than one specific marker allele or a specific haplotype can be indicated by using several sequence-specific nucleic acid probes, each being specific for a particular allele. A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample.

To diagnose a susceptibility to a condition selected from abdominal aortic aneurysm, myocardial infarction, peripheral arterial disease and venous thromboembolism, a hybridization sample can be formed by contacting the test sample, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 10, 15, 30, 50, 100, 250 or 500 nucleotides in length that is sufficient to specifically hybridize under stringent conditions to appropriate mRNA or genomic DNA. For example, the nucleic acid probe can comprise all or a portion of the nucleotide sequence of a gene such as DAB2IP, and/or an LD block as described herein, optionally comprising at least one allele of a marker described herein, or the probe can be the complementary sequence of such a sequence. In a particular embodiment, the nucleic acid probe is a portion of the nucleotide sequence of a gene selected from the above group and/or an LD block as described herein, optionally comprising at least one allele of a marker described herein, or at least one allele of a polymorphic marker of a haplotype comprising at least one polymorphic marker described herein, or the probe can be the complementary sequence of such a sequence. Other suitable probes for use in the diagnostic assays of the invention are described herein. Hybridization can be performed by methods well known to the person skilled in the art (see, e.g., Current Protocols in Molecular Biology, Ausubel et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe. The process can be repeated for any markers of the invention, or markers that make up a haplotype of the invention, or multiple probes can be used concurrently to detect more than one marker alleles at a time.

Additionally, or alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen et al., Bioconjug. Chem. 5:3-7 (1994)). The PNA probe can be designed to specifically hybridize to a molecule in a sample suspected of containing one or more of the marker alleles or haplotypes that are associated with eosinophilia, asthma, myocardial infarction, and/or hypertension.

In one embodiment of the invention, a test sample containing genomic DNA obtained from the subject is collected and the polymerase chain reaction (PCR) is used to amplify a fragment comprising one or more markers of the invention. As described herein, identification of particular marker alleles can be accomplished using a variety of methods. In another embodiment, determination of a susceptibility is accomplished by expression analysis, for example using quantitative PCR (kinetic thermal cycling). This technique can, for example, utilize commercially available technologies, such as TaqMan® (Applied Biosystems, Foster City, Calif.). The technique can for example assess the presence of an alteration in the expression or composition of a polypeptide or splicing variant(s) that is encoded by a nucleic acid associated described herein. Alternatively, this technique may assess expression levels of genes or particular splice variants of genes, that are affected by one or more of the variants described herein. Further, the expression of the variant(s) can be quantified as physically or functionally different.

In another method of the invention, analysis by restriction digestion can be used to detect a particular allele if the allele results in the creation or elimination of a restriction site relative to a reference sequence. Restriction fragment length polymorphism (RFLP) analysis can be conducted, e.g., as described in Current Protocols in Molecular Biology, supra. The digestion pattern of the relevant DNA fragment indicates the presence or absence of the particular allele in the sample.

Sequence analysis can also be used to detect specific alleles. In one embodiment, determination of the presence or absence of a particular marker allele or haplotype comprises sequence analysis of a test sample of DNA or RNA obtained from a subject or individual. PCR or other appropriate methods can be used to amplify a portion of a nucleic acid containing polymorphic markers and the presence of a specific allele can then be detected directly by sequencing the polymorphic site (or multiple polymorphic sites in a haplotype) of the genomic DNA in the sample.

In certain embodiments, nucleic acid sequence data is obtained by a method that comprises at least one procedure selected from (i) amplification of nucleic acid from the biological sample; (ii) hybridization assay using a nucleic acid probe and nucleic acid from the biological sample; and (iii) hybridization assay using a nucleic acid probe and nucleic acid obtained by amplification of the biological sample.

Allele-specific oligonucleotides can also be used to detect the presence of a particular allele in a nucleic acid. An “allele-specific oligonucleotide” (also referred to herein as an “allele-specific oligonucleotide probe”) is an oligonucleotide of any suitable size, for example an oligonucleotide of approximately 10-50 base pairs or approximately 15-30 base pairs, that specifically hybridizes to a nucleic acid which contains a specific allele at a polymorphic site (e.g., a polymorphic marker). An allele-specific oligonucleotide probe that is specific for one or more particular alleles at polymorphic markers can be prepared using standard methods (see, e.g., Current Protocols in Molecular Biology, supra). PCR can be used to amplify the desired region. Specific hybridization of an allele-specific oligonucleotide probe to DNA from a subject is indicative of the presence of a specific allele at a polymorphic site (see, e.g., Gibbs et al., Nucleic Acids Res. 17:2437-2448 (1989) and WO 93/22456).

With the addition of analogs such as locked nucleic acids (LNA5), the size of primers and probes can be reduced to as few as 8 bases. LNA5 are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures (Tm) of 64° C. and 74° C. when in complex with complementary DNA or RNA, respectively, as opposed to 28° C. for both DNA and RNA for the corresponding DNA nonamer. Substantial increases in Tm are also obtained when LNA monomers are used in combination with standard DNA or RNA monomers. For primers and probes, depending on where the LNA monomers are included (e.g., the 3′ end, the 5′ end, or in the middle), the Tm could be increased considerably. It is therefore contemplated that in certain embodiments, LNA5 are used to detect particular alleles at polymorphic sites associated with particular vascular conditions, as described herein.

In certain embodiments, arrays of oligonucleotide probes that are complementary to target nucleic acid sequence segments from a subject, can be used to identify polymorphisms in a nucleic acid. For example, an oligonucleotide array can be used. Oligonucleotide arrays typically comprise a plurality of different oligonucleotide probes that are coupled to a surface of a substrate in different known locations. These arrays can generally be produced using mechanical synthesis methods or light directed synthesis methods that incorporate a combination of photolithographic methods and solid phase oligonucleotide synthesis methods, or by other methods known to the person skilled in the art (see, e.g., Bier et al., Adv Biochem Eng Biotechnol 109:433-53 (2008); Hoheisel, Nat Rev Genet. 7:200-10 (2006); Fan et al., Methods Enzymol 410:57-73 (2006); Raqoussis & Elvidge, Expert Rev Mol Diagn 6:145-52 (2006); Mockler et al., Genomics 85:1-15 (2005), and references cited therein, the entire teachings of each of which are incorporated by reference herein). Many additional descriptions of the preparation and use of oligonucleotide arrays for detection of polymorphisms can be found, for example, in U.S. Pat. No. 6,858,394, U.S. Pat. No. 6,429,027, U.S. Pat. No. 5,445,934, U.S. Pat. No. 5,700,637, U.S. Pat. No. 5,744,305, U.S. Pat. No. 5,945,334, U.S. Pat. No. 6,054,270, U.S. Pat. No. 6,300,063, U.S. Pat. No. 6,733,977, U.S. Pat. No. 7,364,858, EP 619 321, and EP 373 203, the entire teachings of which are incorporated by reference herein.

Also, standard techniques for genotyping can be used to detect particular marker alleles, such as fluorescence-based techniques (e.g., Chen et al., Genome Res. 9(5): 492-98 (1999); Kutyavin et al., Nucleic Acid Res. 34:e128 (2006)), utilizing PCR, LCR, Nested PCR and other techniques for nucleic acid amplification. Specific commercial methodologies available for SNP genotyping include, but are not limited to, TaqMan genotyping assays and SNPlex platforms (Applied Biosystems), gel electrophoresis (Applied Biosystems), mass spectrometry (e.g., MassARRAY system from Sequenom), minisequencing methods, real-time PCR, Bio-Plex system (BioRad), CEQ and SNPstream systems (Beckman), array hybridization technology (e.g., Affymetrix GeneChip; Perlegen), BeadArray Technologies (e.g., Illumina GoldenGate and Infinium assays), array tag technology (e.g., Parallele), and endonuclease-based fluorescence hybridization technology (Invader; Third Wave). Some of the available array platforms, including Affymetrix SNP Array 6.0 and Illumina CNV370-Duo and 1M BeadChips, include SNPs that tag certain copy number variations (CNVs). This allows detection of CNVs via surrogate SNPs included in these platforms. Thus, by use of these or other methods available to the person skilled in the art, one or more alleles at polymorphic markers, including microsatellites, SNPs or other types of polymorphic markers, can be identified.

Direct sequence analysis can be of a nucleic acid of a biological sample obtained from the human individual for which a susceptibility is being determined. The biological sample can be any sample containing nucleic acid (e.g., genomic DNA) obtained from the human individual. For example, the biological sample can be a blood sample, a serum sample, a leukapheresis sample, an amniotic fluid sample, a cerbrospinal fluid sample, a hair sample, a tissue sample from skin, muscle, buccal, or conjuctival mucosa, placenta, gastrointestinal tract, or other organs, a semen sample, a urine sample, a saliva sample, a nail sample, a tooth sample, and the like.

In specific embodiments of the invention, obtaining nucleic acid sequence data comprises obtaining nucleic acid sequence information from a preexisting record, e.g., a preexisting medical record comprising genotype information of the human individual. For example, direct sequence analysis of the allele of the polymorphic marker can be accomplished by mining a pre-existing genotype dataset for the sequence of the allele of the polymorphic marker.

### Indirect Analysis

Nucleic acid sequence data may also be obtained through indirect analysis of the nucleic acid sequence of the allele of the polymorphic marker. For example, the allele could be one which leads to the expression of a variant protein comprising an altered amino acid sequence, as compared to the non-variant (e.g., wild-type) protein, due to one or more amino acid substitutions, deletions, or insertions, or truncation (due to, e.g., splice variation). In this instance, nucleic acid sequence data about the allele of the polymorphic marker can be obtained through detection of the amino acid substitution of the variant protein. Methods of detecting variant proteins are known in the art. For example, direct amino acid sequencing of the variant protein followed by comparison to a reference amino acid sequence can be used. Alternatively, SDS-PAGE followed by gel staining can be used to detect variant proteins of different molecular weights. Also, Immunoassays, e.g., immunofluorescent immunoassays, immunoprecipitations, radioimmunoasays, ELISA, and Western blotting, in which an antibody specific for an epitope comprising the variant sequence among the variant protein and non-variant or wild-type protein can be used.

It is also possible, for example, for the variant protein to demonstrate altered (e.g., upregulated or downregulated) biological activity, in comparison to the non-variant or wild-type protein. The biological activity can be, for example, a binding activity or enzymatic activity. In this instance, nucleic acid sequence data about the allele of the polymorphic marker can be obtained through detection of the altered biological activity. Methods of detecting binding activity and enzymatic activity are known in the art and include, for instance, ELISA, competitive binding assays, quantitative binding assays using instruments such as, for example, a Biacore® 3000 instrument, chromatographic assays, e.g., HPLC and TLC.

Alternatively or additionally, the polymorphic variant (the allele of the polymorphic marker) could lead to an altered expression level, e.g., an increased expression level of an mRNA or protein, a decreased expression level of an mRNA or protein. Nucleic acid sequence data about the allele of the polymorphic marker can, in these instances, be obtained through detection of the altered expression level. Methods of detecting expression levels are known in the art. For example, ELISA, radioimmunoassays, immunofluorescence, and Western blotting can be used to compare the expression of protein levels. Alternatively, Northern blotting can be used to compare the levels of mRNA. These processes are described in Sambrook et al., Molecular Cloning: A Laboratory Manual, 3^{rd} ed. Cold Spring Harbor Laboratory Press, Cold Spring Harbor, N.Y. (2001).

The indirect sequence analysis can be of the nucleic acid (e.g., DNA, mRNA) or protein of a biological sample obtained from the human individual for which a susceptibility is being determined. The biological sample can be any nucleic acid or protein containing sample obtained from the human individual. For example, the biological sample can be any of the biological samples described herein.

In view of the foregoing, analyzing the sequence of the at least one polymorphic marker can comprise determining the presence or absence of at least one allele of the at least one polymorphic marker, or it can comprise analyzing the sequence of the polymorphic marker of a particular sample. Further, analyzing the sequence of the at least one polymorphic marker can comprise determining the presence or absence of an amino acid substitution in the amino acid sequence encoded by the polymorphic marker of at least one gene of the group, or it can comprise obtaining a biological sample from the human individual and analyzing the amino acid sequence encoded by at least one gene of the group.

### Number of Polymorphic Markers/Genes Analyzed

With regard to the methods and other aspects of the invention described herein, sequence data about any number of polymorphic markers may be suitably analyzed. For example, analysis of sequence data may be performed for about at least 1, 2, 3, 4, 5, 6, 7, 8, 9, 10, 15, 20, 25, 30, 40, 50, 100, 500, 1000, 10,000 or more polymorphic markers. The markers can be independent and/or the markers may be in linkage disequilibrium. The markers may also form a haplotype or reside on the same haplotype. The polymorphic markers can be the ones of a group specified herein or they can be different polymorphic markers that are not listed herein, including, for example, polymorphic markers in linkage disequilibrium with the markers described herein, or other markers that have been previously identified as associating with a vascular condition selected from the group consisting of abdominal aortic aneurysm, myocardial infarction, peripheral arterial disease and venous thromboembolism. In a specific embodiment, sequence data about at least two polymorphic markers is analyzed. In certain embodiments, each of the markers may be associated with, or located within, a different gene. For example, in some instances, if nucleic acid data about a human individual identifying at least one allele of a polymorphic marker is analyzed, then a risk assessment method comprises identifying at least one allele of at least one polymorphic marker. Also, for example, the method can comprise analyzing sequence data about a human individual identifying alleles of multiple, independent markers or haplotypes, which are not in linkage disequilibrium.

### Linkage Disequilibrium

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrance of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurence of each allele or haplotype in the population. For populations of diploids, e.g., human populations, individuals will typically have two alleles for each genetic element (e.g., a marker, haplotype or gene).

Many different measures have been proposed for assessing the strength of linkage disequilibrium (LD; reviewed in Devlin, B. & Risch, N., Genomics 29:311-22 (1995)). Most capture the strength of association between pairs of biallelic sites. Two important pairwise measures of LD are r² (sometimes denoted Δ²) and |D′| (Lewontin, R., Genetics 49:49-67 (1964); Hill, W. G. & Robertson, A. Theor. Appl. Genet. 22:226-231 (1968)). Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots.

For the methods described herein, a significant r² value can be at least 0.1 such as at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, 0.99 or 1.0. In one specific embodiment of invention, the significant r² value can be at least 0.2. Alternatively, linkage disequilibrium as described herein, refers to linkage disequilibrium characterized by values of |D′| of at least 0.2, such as 0.3, 0.4, 0.5, 0.6, 0.7, 0.8, 0.85, 0.9, 0.95, 0.96, 0.97, 0.98, 0.99. Thus, linkage disequilibrium represents a correlation between alleles of distinct markers. It is measured by correlation coefficient or |D′| (r² up to 1.0 and |D′| up to 1.0). Linkage disequilibrium can be determined in a single human population, as defined herein, or it can be determined in a collection of samples comprising individuals from more than one human population. In one embodiment of the invention, LD is determined in a sample from one or more of the HapMap populations. These include samples from the Yoruba people of Ibadan, Nigeria (YR1), samples from individuals from the Tokyo area in Japan (JPT), samples from individuals Beijing, China (CHB), and samples from U.S. residents with northern and western European ancestry (CEU), as described (The International HapMap Consortium, Nature 426:789-796 (2003)). In one such embodiment, LD is determined in the Caucasian CEU population of the HapMap samples. In another embodiment, LD is determined in the African YRO population. In yet another embodiment, LD is determined in samples from the Icelandic population.

If all polymorphisms in the genome were independent at the population level (i.e., no LD between polymorphisms), then every single one of them would need to be investigated in association studies, to assess all different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). More recently, a fine-scale map of recombination rates and corresponding hotspots across the human genome has been generated (Myers, S., et al., Science 310:321-32324 (2005); Myers, S. et al., Biochem Soc Trans 34:526530 (2006)). The map reveals the enormous variation in recombination across the genome, with recombination rates as high as 10-60 cM/Mb in hotspots, while closer to 0 in intervening regions, which thus represent regions of limited haplotype diversity and high LD. The map can therefore be used to define haplotype blocks/LD blocks as regions flanked by recombination hotspots. As used herein, the terms “haplotype block” or “LD block” includes blocks defined by any of the above described characteristics, or other alternative methods used by the person skilled in the art to define such regions.

Haplotype blocks (LD blocks) can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the invention. One or more causative (functional) variants or mutations may reside within the region found to be associating to the disease or trait. The functional variant may be another SNP, a tandem repeat polymorphism (such as a minisatellite or a microsatellite), a transposable element, or a copy number variation, such as an inversion, deletion or insertion. Such variants in LD with other variants used to detect an association to a disease or trait (e.g., the variants described herein to be associated with risk of abdominal aortic aneurysm, myocardial infarction, peripheral arterial disease and venous thromboembolism) may confer an even higher relative risk (RR) or odds ratio (OR) than observed for the tagging markers used to detect the association. The invention thus refers to the markers used for detecting association to the disease, as described herein, as well as markers in linkage disequilibrium with the markers. Thus, in certain embodiments of the invention, markers that are in LD with the markers and/or haplotypes of the invention, as described herein, may be used as surrogate markers. The surrogate markers have in one embodiment relative risk (RR) and/or odds ratio (OR) values smaller than for the markers or haplotypes initially found to be associating with the disease, as described herein. In other embodiments, the surrogate markers have RR or OR values greater than those initially determined for the markers initially found to be associating with the disease, as described herein. An example of such an embodiment would be a rare, or relatively rare (<10% allelic population frequency) variant in LD with a more common variant (>10% population frequency) initially found to be associating with the disease, such as the variants described herein. Identifying and using such markers for detecting the association discovered by the inventors as described herein can be performed by routine methods well known to the person skilled in the art, and are therefore within the scope of the invention.

Markers in linkage disequilibrium with the markers shown herein to be associated with a condition selected from the group consisting of abdominal aortic aneurysm, myocardial infarction, peripheral arterial disease and venous thromboembolism are, by necessity, also associated with the condition. Thus, in one embodiment, surrogate markers of rs7025486 as shown in Table 1 are also associated with the condition. This fact is obvious to the skilled person, who thus knows that any surrogate marker may be suitably selected to test an association determined for any particular anchor marker. The stronger the linkage disequilibrium to the anchor marker, the better the surrogate, and thus the mores similar the association detected by the surrogate is expected to be to the association detected by the anchor marker. Markers with values of r² equal to 1 are perfect surrogates for the at-risk variants, i.e. genotypes for one marker perfectly predicts genotypes for the other. In other words, the surrogate will, by necessity, give exactly the same association data to any particular disease as the anchor marker. Markers with smaller values of r² than 1 can also be surrogates for the at-risk anchor variant. Surrogate markers with smaller values of r² than 1 may be variants with risk values smaller than for the anchor marker. Alternatively, such surrogate markers may represent variants with relative risk values as high as or possibly even higher than the at-risk variant. In this scenario, the at-risk variant identified may not be the functional variant itself, but is in this instance in linkage disequilibrium with the true functional variant. The functional variant may be a SNP, but may also for example be a tandem repeat, such as a minisatellite or a microsatellite, a transposable element (e.g., an A/u element), or a structural alteration, such as a deletion, insertion or inversion (sometimes also called copy number variations, or CNVs). The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation identify and genotype surrogate markers in linkage disequilibrium with the markers and/or haplotypes as described herein.

In view of the foregoing, markers in linkage disequilibrium with a polymorphic marker associated with a condition selected from the group consisting of abdominal aortic aneurysm, myocardial infarction, peripheral arterial disease and venous thromboembolism may be one of the surrogate markers listed in Table 1.

TABLE 1(A)
Surrogate markers of anchor marker rs7025486-A based on
Caucasian HapMap (v24) dataset. Shown are marker names,
position in NCBI build 36, predicted Risk Allele correlated with
the A allele of rs7025486, values of r² and Seq ID number
of the surrogate.

| | Pos. In NCBI Build | | | |
|---|---|---|---|---|
| SNP | 36 | Risk Allele | r² | SEQ ID NO: |
| rs584985 | 123437111 | C | 0.21 | 48 |
| rs2777310 | 123439577 | A | 0.29 | 36 |
| rs2797348 | 123439831 | G | 0.26 | 42 |
| rs12352132 | 123440666 | G | 0.36 | 21 |
| rs2777308 | 123442163 | A | 0.31 | 35 |
| rs2797347 | 123442393 | C | 0.25 | 41 |
| rs1003016 | 123443141 | C | 0.26 | 1 |
| rs12553641 | 123447797 | A | 0.27 | 23 |
| rs12554667 | 123450426 | G | 0.30 | 25 |
| rs10760182 | 123452782 | G | 0.22 | 3 |
| rs10818577 | 123453012 | C | 0.89 | 5 |
| rs10818578 | 123454572 | G | 1.00 | 6 |
| rs10818579 | 123454726 | A | 0.96 | 7 |
| rs10818580 | 123454843 | A | 1.00 | 8 |
| rs10985344 | 123456761 | A | 1.00 | 13 |
| rs885150 | 123459994 | C | 1.00 | 58 |
| rs10818583 | 123462082 | A | 1.00 | 10 |
| rs7025486 | 123462224 | A | 1.00 | 52 |
| rs10985349 | 123465064 | T | 0.76 | 15 |
| rs10818589 | 123549071 | T | 0.26 | 11 |
| rs10116069 | 123567781 | T | 0.26 | 2 |
| rs2416834 | 123591519 | C | 0.23 | 34 |

TABLE 1B
Surrogate markers of rs7025486-A bases on Caucasian samples in the
publically available 1000 Genomes project
(http://www.1000genomes.org). Markers that have not been assigned
rs names are identified by their position in NCBI Build 36 of the human
genome assembly. Shown are marker names, position in NCBI build 36,
predicted Risk Allele correlated with allele A of anchor marker,
values of r² and P-value and finally their Seq ID NO.

| | Pos. in | | | | | SEQ |
|---|---|---|---|---|---|---|
| | NCBI | Risk | | | | ID |
| SNP | Build 36 | Allele | D′ | r² | P-value | NO: |
| rs878708 | 123385920 | A | 0.59 | 0.22 | 0.000071 | 57 |
| rs669128 | 123392581 | C | 0.63 | 0.23 | 0.000053 | 51 |
| rs2009828 | 123396379 | G | 0.59 | 0.22 | 0.000071 | 32 |
| rs2777320 | 123397291 | A | 0.59 | 0.22 | 0.000071 | 39 |
| rs4836858 | 123398134 | T | 0.59 | 0.22 | 0.000071 | 46 |
| rs7038469 | 123400367 | T | 0.59 | 0.22 | 0.000071 | 53 |
| rs2777319 | 123402323 | T | 0.59 | 0.22 | 0.000071 | 38 |
| rs7869336 | 123405034 | T | 0.65 | 0.27 | 0.000011 | 55 |
| rs2797349 | 123436574 | A | 0.77 | 0.17 | 0.001 | 43 |
| rs584985 | 123437111 | C | 0.55 | 0.28 | 0.000015 | 48 |
| rs2777311 | 123437209 | C | 0.55 | 0.25 | 0.000053 | 37 |
| rs2777310 | 123439577 | A | 0.67 | 0.32 | 0.0000016 | 36 |
| rs2797348 | 123439831 | G | 0.67 | 0.32 | 0.0000016 | 42 |
| rs12352132 | 123440666 | G | 0.7 | 0.38 | 0.00000015 | 21 |
| rs62575880 | 123441329 | A | 0.7 | 0.38 | 0.00000015 | 50 |
| rs2777308 | 123442163 | A | 0.63 | 0.3 | 0.0000042 | 35 |
| rs2797347 | 123442393 | C | 0.77 | 0.37 | 0.00000012 | 41 |
| rs1003016 | 123443141 | C | 0.77 | 0.37 | 0.00000012 | 1 |
| s.123444035 | 123444035 | G | 0.67 | 0.32 | 0.0000016 | 59 |
| s.123444070 | 123444070 | T | 0.83 | 0.53 | 3.9E−10 | 60 |
| rs1768732 | 123445098 | C | 0.77 | 0.37 | 0.00000012 | 29 |
| s.123445547 | 123445547 | A | 0.64 | 0.32 | 0.0000016 | 61 |
| s.123446322 | 123446322 | A | 0.69 | 0.2 | 0.00061 | 62 |
| rs7465724 | 123446680 | C | 0.67 | 0.32 | 0.0000016 | 54 |
| s.123446681 | 123446681 | A | 0.63 | 0.38 | 0.00000028 | 63 |
| s.123447265 | 123447265 | A | 0.63 | 0.24 | 0.00019 | 64 |
| rs12553641 | 123447797 | A | 0.63 | 0.38 | 0.00000028 | 23 |
| s.123449587 | 123449587 | T | 0.79 | 0.5 | 1.4E−09 | 65 |
| rs12554639 | 123450214 | A | 0.69 | 0.4 | 0.00000013 | 24 |
| rs12554667 | 123450426 | G | 1 | 0.27 | 9.3E−11 | 25 |
| s.123451318 | 123451318 | G | 0.91 | 0.76 | 1E−15 | 66 |
| s.123451324 | 123451324 | T | 1 | 0.25 | 0.000000055 | 67 |
| s.123451615 | 123451615 | T | 1 | 0.25 | 0.000000055 | 68 |
| s.123451617 | 123451617 | A | 1 | 0.04 | 0.026 | 69 |
| rs10818576 | 123452769 | G | 0.78 | 0.61 | 3.1E−12 | 4 |
| rs10760182 | 123452782 | G | 0.81 | 0.21 | 0.000043 | 3 |
| rs10818577 | 123453012 | C | 0.91 | 0.76 | 1E−15 | 5 |
| rs10818578 | 123454572 | G | 0.96 | 0.88 | 1.30E+19 | 6 |
| rs10818579 | 123454726 | A | 0.96 | 0.88 | 1.30E+19 | 7 |
| rs10818580 | 123454843 | A | 0.96 | 0.88 | 1.30E+19 | 8 |
| s.123455512 | 123455512 | C | 0.96 | 0.88 | 1.30E+19 | 70 |
| rs10818582 | 123455826 | G | 1 | 0.3 | 1.1E−11 | 9 |
| rs10985344 | 123456761 | A | 0.96 | 0.88 | 1.30E+19 | 13 |
| rs62572789 | 123458131 | A | 0.96 | 0.88 | 1.30E+19 | 49 |
| rs12380555 | 123458318 | C | 0.96 | 0.88 | 1.30E+19 | 22 |
| rs10985347 | 123459238 | T | 0.96 | 0.92 | 2.30E+21 | 14 |
| s.123459543 | 123459543 | C | 0.96 | 0.88 | 1.30E+19 | 71 |
| rs885150 | 123459994 | C | 1 | 0.92 | 5.90E+28 | 58 |
| s.123461786 | 123461786 | C | 1 | 0.85 | 1.80E+26 | 72 |
| rs10818583 | 123462082 | A | 1 | 1 | 4.50E+32 | 10 |
| s.123462115 | 123462115 | G | 1 | 0.2 | 0.000000015 | 73 |
| rs1984038 | 123464010 | C | 0.4 | 0.03 | 0.21 | 31 |
| rs10985349 | 123465064 | T | 1 | 0.63 | 2.6E−18 | 15 |
| rs1984037 | 123465526 | C | 0.59 | 0.08 | 0.023 | 30 |
| s.123469017 | 123469017 | C | 1 | 0.7 | 2.50E+20 | 74 |
| s.123472214 | 123472214 | A | 1 | 0.28 | 0.000000011 | 75 |
| rs12000685 | 123473367 | C | 0.55 | 0.04 | 0.10 | 18 |
| rs12000723 | 123473502 | C | 0.55 | 0.04 | 0.10 | 19 |
| rs35661033 | 123477422 | C | 0.43 | 0.02 | 0.25 | 44 |
| s.123479977 | 123479977 | A | 0.73 | 0.25 | 0.00013 | 76 |
| rs1571804 | 123488592 | T | 0.22 | 0.04 | 0.17 | 28 |
| s.123591409 | 123591409 | C | 0.76 | 0.21 | 0.00056 | 77 |
| rs10985475 | 123785952 | T | 0.45 | 0.01 | 0.41 | 17 |

In one preferred embodiment, markers in LD are surrogate markers with r²>0.2 to the anchor marker in Caucasians. For example, surrogate markers of rs7025486 may suitably be selected from the group consisting of rs584985, rs2777310, rs2797348, rs12352132, rs2777308, rs2797347, rs1003016, rs12553641, rs12554667, rs10760182, rs10818577, rs10818578, rs10818579, rs10818580, rs10985344, rs885150, rs10818583, rs7025486, rs10985349, rs10818589, rs10116069, rs2416834, rs878708, rs669128, rs2009828, rs2777320, rs4836858, rs7038469, rs2777319, rs7869336, rs2797349, rs2777311, rs62575880, s.123444035, s.123444070, rs1768732, s.123445547, s.123446322, rs7465724, s.123446681, s.123447265, s.123449587, rs12554639, s.123451318, s.123451324, s.123451615, s.123451617, rs10818576, s.123455512, rs62572789, rs12380555, rs10985347, s.123459543, rs885150, s.123461786, s.123462115, rs1984038, rs1984037, s.123469017, s.123472214, rs12000685, rs12000723, rs35661033, s.123479977, rs1571804, s.123591409, and rs10985475, which are the markers listed in Table 1. In another embodiment, surrogate markers of rs7025486 are selected from the group consisting of rs584985, rs2777310, rs2797348, rs12352132, rs2777308, rs2797347, rs1003016, rs12553641, rs12554667, rs10760182, rs10818577, rs10818578, rs10818579, rs10818580, rs10985344, rs885150, rs10818583, rs7025486, rs10985349, rs10818589, rs10116069, and rs2416834, which are the markers listed in Table 1A. In another embodiment, surrogate markers of rs7025486 are selected from the group consisting of rs878708, rs669128, rs2009828, rs2777320, rs4836858, rs7038469, rs2777319, rs7869336, rs2797349, rs584985, rs2777311, rs2777310, rs2797348, rs12352132, rs62575880, rs2777308, rs2797347, rs1003016, s.123444035, s.123444070, rs1768732, s.123445547, s.123446322, rs7465724, s.123446681, s.123447265, rs12553641, s.123449587, rs12554639, rs12554667, s.123451318, s.123451324, s.123451615, s.123451617, rs10818576, rs10760182, rs10818577, rs10818578, rs10818579, rs10818580, s.123455512, rs10818582, rs10985344, rs62572789, rs12380555, rs10985347, s.123459543, rs885150, s.123461786, rs10818583, s.123462115, rs1984038, rs10985349, rs1984037, s.123469017, s.123472214, rs12000685, rs12000723, rs35661033, s.123479977, rs1571804, s.123591409, and rs10985475, which are the markers listed in Table 16. In another embodiment, suitable surrogate markers are those markers that are in LD with rs7025486 characterized by values of r² to rs7025486>0.5. In one such embodiment, suitable surrogate markers are selected from the group consisting of rs10818578, rs10818577, rs10818579, rs10818580, rs10985344, rs885150, rs10818583, rs7025486, rs10985349, s.123444070, s.123451318, rs10818576, rs10818578, s.123455512, rs62572789, rs12380555, rs10985347, s.123459543, rs885150, s.123461786, rs10818583, rs10985349, and s.123469017. In yet another preferred embodiment, suitable surrogate markers in LD are markers with r² to the anchor marker >0.8. Such and other surrogate markers based on other suitable cutoff values of r² may be selected from the markers listed in Table 1, using the values of r² provided in the Table.

In a further embodiment, suitable surrogate markers of rs7025486 are selected from the group consisting of the markers rs878708, rs669128, rs2009828, rs2777320, rs4836858, rs7038469, rs2777319, rs584985, rs2777311, rs2777310, rs2797348, rs12352132, rs62575880, rs2777308, rs2797347, rs1003016, s.123444035, s.123444070, rs1768732, s.123445547, s.123446322, rs7465724, s.123446681, s.123447265, rs12553641, s.123449587, rs12554639, rs12554667, s.123451318, s.123451324, rs10818576, rs10760182, rs10818577, rs10818578, rs10818579, rs10818580, s.123455512, rs10818582, rs10985344, rs62572789, rs12380555, rs10985347, s.123459543, rs885150, s.123461786, rs10818583, s.123462115, rs10985349, s.123469017, s.123472214, s.123479977, rs10818589, rs10116069, and rs2416834, which are the markers listed in Table 11.

In one embodiment, surrogate markers of rs7025486 selected from the group consisting of rs584985, rs2777311, rs2777310, rs2797348, rs12352132, rs62575880, rs2777308, rs2797347, rs1003016, s.123444035, s.123444070, rs1768732, s.123445547, rs7465724, s.123446681, s.123447265, rs12553641, s.123449587, rs12554639, rs12554667, s.123451318, rs10818576, rs10760182, rs10818577, rs10818578, rs10818579, rs10818580, s.123455512, rs10818582, rs10985344, rs62572789, rs12380555, rs10985347, s.123459543, rs885150, s.123461786, rs10818583, rs10985349, s.123469017 are useful in diagnostic applications that relate to early onset Myocardial Infarction, including but not limited to determination of susceptibility of early onset Myocardial Infarction.

In one embodiment, surrogate markers of rs7025486 selected from the group consisting of s.123446322, s.123447265, s.123451318, rs10818576, rs10818577, rs10818578, rs10818579, rs10818580, s.123455512, rs10818582, rs10985344, rs62572789, rs12380555, rs10985347, s.123459543, rs885150, s.123461786, rs10818583, rs10985349, s.123469017 are useful in diagnostic applications that relate to Peripheral Arterial Disease, including but not limited to determination of susceptibility of Peripheral Arterial Disease.

In one embodiment, surrogate markers of rs7025486 selected from the group consisting of s.123451318, s.123451324, rs10818576, rs10818577, rs10818578, rs10818579, rs10818580, s.123455512, rs10985344, rs62572789, rs12380555, rs10985347, s.123459543, rs885150, s.123461786, rs10818583, rs10985349, s.123469017, s.123472214, s.123479977 are useful in diagnostic applications that relate to VTE, including but not limited to determination of susceptibility of VTE.

In one embodiment, surrogate markers of rs7025486 selected from the group consisting of s.123447265, s.123451318, s.123451324, rs10818576, rs10818577, rs10818578, rs10818579, rs10818580, s.123455512, rs10985344, rs62572789, rs12380555, rs10985347, s.123459543, rs885150, s.123461786, rs10818583, rs10985349, s.123469017 are useful in diagnostic applications that relate to PE, including but not limited to determination of susceptibility of PE.

### Imputing Genotypes

It is possible to impute or predict genotypes for un-genotyped relatives of genotyped individuals.

For every un-genotyped case, it is possible to calculate the probability of the genotypes of its relatives given its four possible phased genotypes. In practice it may be preferable to include only the genotypes of the case's parents, children, siblings, half-siblings (and the half-sibling's parents), grand-parents, grand-children (and the grand-children's parents) and spouses. It will be assumed that the individuals in the small sub-pedigrees created around each case are not related through any path not included in the pedigree. It is also assumed that alleles that are not transmitted to the case have the same frequency—the population allele frequency. Let us consider a SNP marker with the alleles A and G. The probability of the genotypes of the case's relatives can then be computed by:

$$Pr ( genotypes of relatives ; θ ) = {\sum }_{h ∈ \{ AA , AG , GA , GG \}} Pr ( h ; θ ) Pr ( genotypes of relatives \vert h ) ,$$

where θ denotes the A allele's frequency in the cases. Assuming the genotypes of each set of relatives are independent, this allows us to write down a likelihood function for θ:

$$L ( θ ) = {\prod }_{i} Pr ( genotypes of relatives of case i ; θ ) . \qquad (* )$$

This assumption of independence is usually not correct. Accounting for the dependence between individuals is a difficult and potentially prohibitively expensive computational task. The likelihood function in (*) may be thought of as a pseudolikelihood approximation of the full likelihood function for 6 which properly accounts for all dependencies. In general, the genotyped cases and controls in a case-control association study are not independent and applying the case-control method to related cases and controls is an analogous approximation. The method of genomic control (Devlin, B. et al., Nat Genet. 36, 1129-30; author reply 1131 (2004)) has proven to be successful at adjusting case-control test statistics for relatedness. We therefore apply the method of genomic control to account for the dependence between the terms in our pseudolikelihood and produce a valid test statistic.

Fisher's information can be used to estimate the effective sample size of the part of the pseudolikelihood due to un-genotyped cases. Breaking the total Fisher information, I, into the part due to genotyped cases, I_{g}, and the part due to ungenotyped cases, Iᵤ, I=I_{g}+Iᵤ, and denoting the number of genotyped cases with N, the effective sample size due to the un-genotyped cases is estimated by

$$\frac{{I}_{u}}{{I}_{g}} N .$$

It is also possible to impute genotypes for markers with no genotype data. For example, using the IMPUTE (Marchini, J. et al. Nat Genet. 39:906-13 (2007)) software and the HapMap (NCBI Build 36 (db126b)) CEU data as reference (Frazer, K. A., et al. Nature 449:851-61 (2007)) it is possible to impute ungenotyped markers. This can be useful for extending genotype coverage, if the CEU dataset has been genotyped.

### Haplotype Analysis

The frequencies of haplotypes in patient and control groups can be estimated using an expectation-maximization algorithm (Dempster A. et al., J. R. Stat. Soc. B, 39:1-38 (1977)). An implementation of this algorithm that can handle missing genotypes and uncertainty with the phase can be used. Under the null hypothesis, the patients and the controls are assumed to have identical frequencies. Using a likelihood approach, an alternative hypothesis is tested, where a candidate at-risk-haplotype, which can include the markers described herein, is allowed to have a higher frequency in patients than controls, while the ratios of the frequencies of other haplotypes are assumed to be the same in both groups. Likelihoods are maximized separately under both hypotheses and a corresponding 1-df likelihood ratio statistic is used to evaluate the statistical significance.

To look for at-risk and protective markers and haplotypes within a susceptibility region, for example within an LD block, association of all possible combinations of genotyped markers within the region is studied. The combined patient and control groups can be randomly divided into two sets, equal in size to the original group of patients and controls. The marker and haplotype analysis is then repeated and the most significant p-value registered is determined. This randomization scheme can be repeated, for example, over 100 times to construct an empirical distribution of p-values. In a preferred embodiment, a p-value of <0.05 is indicative of a significant marker and/or haplotype association.

One general approach to haplotype analysis involves using likelihood-based inference applied to NEsted MOdels (Gretarsdottir S., et al., Nat. Genet. 35:131-38 (2003)). The method is implemented in the program NEMO, which allows for many polymorphic markers, SNPs and microsatellites. The method and software are specifically designed for case-control studies where the purpose is to identify haplotype groups that confer different risks. It is also a tool for studying LD structures. In NEMO, maximum likelihood estimates, likelihood ratios and p-values are calculated directly, with the aid of the EM algorithm, for the observed data treating it as a missing-data problem.

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. The information measure for haplotype analysis is described in Nicolae and Kong (Technical Report 537, Department of Statistics, University of Statistics, University of Chicago; Biometrics, 60(2):368-75 (2004)) as a natural extension of information measures defined for linkage analysis, and is implemented in NEMO.

### Association Analysis

For single marker association to a disease, the Fisher exact test can be used to calculate two-sided p-values for each individual allele. Correcting for relatedness among patients can be done by extending a variance adjustment procedure previously described (Risch, N. & Teng, J. Genome Res., 8:1273-1288 (1998)) for sibships so that it can be applied to general familial relationships. The method of genomic controls (Devlin, B. & Roeder, K. Biometrics 55:997 (1999)) can also be used to adjust for the relatedness of the individuals and possible stratification.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

An association signal detected in one association study may be replicated in a second cohort, for example a cohort from a different population (e.g., different region of same country, or a different country) of the same or different ethnicity. The advantage of replication studies is that the number of tests performed in the replication study is usually quite small, and hence the less stringent the statistical measure that needs to be applied. For example, for a genome-wide search for susceptibility variants for a particular disease or trait using 300,000 SNPs, a correction for the 300,000 tests performed (one for each SNP) can be performed. Since many SNPs on the arrays typically used are correlated (i.e., in LD), they are not independent. Thus, the correction is conservative. Nevertheless, applying this correction factor requires an observed P-value of less than 0.05/300,000=1.7×10⁻⁷ for the signal to be considered significant applying this conservative test on results from a single study cohort. Obviously, signals found in a genome-wide association study with P-values less than this conservative threshold (i.e., more significant) are a measure of a true genetic effect, and replication in additional cohorts is not necessary from a statistical point of view. Importantly, however, signals with P-values that are greater than this threshold may also be due to a true genetic effect. The sample size in the first study may not have been sufficiently large to provide an observed P-value that meets the conservative threshold for genome-wide significance, or the first study may not have reached genome-wide significance due to inherent fluctuations due to sampling. Since the correction factor depends on the number of statistical tests performed, if one signal (one SNP) from an initial study is replicated in a second case-control cohort, the appropriate statistical test for significance is that for a single statistical test, i.e., P-value less than 0.05. Replication studies in one or even several additional case-control cohorts have the added advantage of providing assessment of the association signal in additional populations, thus simultaneously confirming the initial finding and providing an assessment of the overall significance of the genetic variant(s) being tested in human populations in general.

The results from several case-control cohorts can also be combined to provide an overall assessment of the underlying effect. The methodology commonly used to combine results from multiple genetic association studies is the Mantel-Haenszel model (Mantel and Haenszel, J Natl Cancer Inst 22:719-48 (1959)). The model is designed to deal with the situation where association results from different populations, with each possibly having a different population frequency of the genetic variant, are combined. The model combines the results assuming that the effect of the variant on the risk of the disease, a measured by the OR or RR, is the same in all populations, while the frequency of the variant may differ between the populations. Combining the results from several populations has the added advantage that the overall power to detect a real underlying association signal is increased, due to the increased statistical power provided by the combined cohorts. Furthermore, any deficiencies in individual studies, for example due to unequal matching of cases and controls or population stratification will tend to balance out when results from multiple cohorts are combined, again providing a better estimate of the true underlying genetic effect.

### Risk Assessment and Diagnostics

Within any given population, there is an absolute risk of developing a disease or trait, defined as the chance of a person developing the specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

### Risk Calculations

The creation of a model to calculate the overall genetic risk involves two steps: i) conversion of odds-ratios for a single genetic variant into relative risk and ii) combination of risk from multiple variants in different genetic loci into a single relative risk value.

Deriving Risk from Odds-Ratios

Most gene discovery studies for complex diseases that have been published to date in authoritative journals have employed a case-control design because of their retrospective setup. These studies sample and genotype a selected set of cases (people who have the specified disease condition) and control individuals. The interest is in genetic variants (alleles) which frequency in cases and controls differ significantly.

The results are typically reported in odds ratios, that is the ratio between the fraction (probability) with the risk variant (carriers) versus the non-risk variant (non-carriers) in the groups of affected versus the controls, i.e. expressed in terms of probabilities conditional on the affection status:

OR=(Pr(c|A)/Pr(nc|A))/(Pr(c|C)/Pr(nc|C))

Sometimes it is however the absolute risk for the disease that we are interested in, i.e. the fraction of those individuals carrying the risk variant who get the disease or in other words the probability of getting the disease. This number cannot be directly measured in case-control studies, in part, because the ratio of cases versus controls is typically not the same as that in the general population. However, under certain assumption, we can estimate the risk from the odds ratio.

It is well known that under the rare disease assumption, the relative risk of a disease can be approximated by the odds ratio. This assumption may however not hold for many common diseases. Still, it turns out that the risk of one genotype variant relative to another can be estimated from the odds ratio expressed above. The calculation is particularly simple under the assumption of random population controls where the controls are random samples from the same population as the cases, including affected people rather than being strictly unaffected individuals. To increase sample size and power, many of the large genome-wide association and replication studies use controls that were neither age-matched with the cases, nor were they carefully scrutinized to ensure that they did not have the disease at the time of the study. Hence, while not exactly, they often approximate a random sample from the general population. It is noted that this assumption is rarely expected to be satisfied exactly, but the risk estimates are usually robust to moderate deviations from this assumption.

Calculations show that for the dominant and the recessive models, where we have a risk variant carrier, “c”, and a non-carrier, “nc”, the odds ratio of individuals is the same as the risk ratio between these variants:

OR=Pr(A|c)/Pr(A|nc)=r

And likewise for the multiplicative model, where the risk is the product of the risk associated with the two allele copies, the allelic odds ratio equals the risk factor:

OR=Pr(A|aa)/Pr(A|ab)=Pr(A|ab)/Pr(A|bb)=r

Here “a” denotes the risk allele and “b” the non-risk allele. The factor “r” is therefore the relative risk between the allele types.

For many of the studies published in the last few years, reporting common variants associated with complex diseases, the multiplicative model has been found to summarize the effect adequately and most often provide a fit to the data superior to alternative models such as the dominant and recessive models.

### The Risk Relative to the Average Population Risk

It is most convenient to represent the risk of a genetic variant relative to the average population since it makes it easier to communicate the lifetime risk for developing the disease compared with the baseline population risk. For example, in the multiplicative model we can calculate the relative population risk for variant “aa” as:

RR(aa)=Pr(A|aa)/Pr(A)=(Pr(A|aa)/Pr(A|bb))/(Pr(A)/Pr(A|bb))=r²/(Pr(aa)r²+Pr(ab)r+Pr(bb))=r²/(p²r²+2pqr+q²)=r2/R

Here “p” and “q” are the allele frequencies of “a” and “b” respectively. Likewise, we get that RR(ab)=r/R and RR(bb)=1/R. The allele frequency estimates may be obtained from the publications that report the odds-ratios and from the HapMap database. Note that in the case where we do not know the genotypes of an individual, the relative genetic risk for that test or marker is simply equal to one.

As an example, for AAA risk, allele A of marker rs7025486 has an allelic OR of 1.21 and a frequency (p) around 0.26 in white populations. The genotype relative risk compared to genotype GG are estimated based on the multiplicative model.

For AA it is 1.21×1.21=1.46; for AG it is simply the OR 1.21, and for GG it is 1.0 by definition.

The frequency of allele G is q=1−p=1−0.26=0.74. Population frequency of each of the three possible genotypes at this marker is:

Pr(AA)=p²=0.07, Pr(CT)=2pq=0.38, and Pr(CC)=q²=0.55

The average population risk relative to genotype GG (which is defined to have a risk of one) is:

R=0.07×1.46+0.38×1.12+0.55×1=1.11

Therefore, the risk relative to the general population (RR) for individuals who have one of the following genotypes at this marker is:

RR(AA)=1.46/1.11=1.32, RR(AG)=1.21/1.11=1.09, RR(GG)=1/1.11=0.90.

Determining Risk from Multiple Markers

A genetic variant associated with a disease or a trait can be used alone to predict the risk of the disease for a given genotype. For a biallelic marker, such as a SNP, there are 3 possible genotypes: homozygote for the at risk variant, heterozygote, and non carrier of the at risk variant. Risk associated with variants at multiple loci can be used to estimate overall risk. For multiple SNP variants, there are k possible genotypes k=3ⁿ×2^{p}; where n is the number autosomal loci and p the number of gonosomal (sex chromosomal) loci. Overall risk assessment calculations for a plurality of risk variants usually assume that the relative risks of different genetic variants multiply, i.e. the overall risk (e.g., RR or OR) associated with a particular genotype combination is the product of the risk values for the genotype at each locus. If the risk presented is the relative risk for a person, or a specific genotype for a person, compared to a reference population with matched gender and ethnicity, then the combined risk is the product of the locus specific risk values and also corresponds to an overall risk estimate compared with the population. If the risk for a person is based on a comparison to non-carriers of the at risk allele, then the combined risk corresponds to an estimate that compares the person with a given combination of genotypes at all loci to a group of individuals who do not carry risk variants at any of those loci. The group of non-carriers of any at risk variant has the lowest estimated risk and has a combined risk, compared with itself (i.e., non-carriers) of 1.0, but has an overall risk, compare with the population, of less than 1.0. It should be noted that the group of non-carriers can potentially be very small, especially for large number of loci, and in that case, its relevance is correspondingly small.

The multiplicative model is a parsimonious model that usually fits the data of complex traits reasonably well. Deviations from multiplicity have been rarely described in the context of common variants for common diseases, and if reported are usually only suggestive since very large sample sizes are usually required to be able to demonstrate statistical interactions between loci.

It is likely that the multiplicative model applied in the case of multiple genetic variant will also be valid in conjugation with non-genetic risk variants assuming that the genetic variant does not clearly correlate with the “environmental” factor. In other words, genetic and non-genetic at-risk variants can be assessed under the multiplicative model to estimate combined risk, assuming that the non-genetic and genetic risk factors do not interact.

When genotypes of many SNP variants are used to estimate the risk for an individual a multiplicative model for risk can generally be assumed. This means that the combined genetic risk relative to the population is calculated as the product of the corresponding estimates for individual markers, e.g. for two markers g1 and g2:

RR(g1,g2)=RR(g1)RR(g2)

The underlying assumption is that the risk factors occur and behave independently, i.e. that the joint conditional probabilities can be represented as products:

Pr(A|g1,g2)=Pr(A|g1)Pr(A|g2)/Pr(A) and Pr(g1,g2)=Pr(g1)Pr(g2)

Obvious violations to this assumption are markers that are closely spaced on the genome, i.e. in linkage disequilibrium, such that the concurrence of two or more risk alleles is correlated. In such cases, we can use so called haplotype modeling where the odds-ratios are defined for all allele combinations of the correlated SNPs.

As is in most situations where a statistical model is utilized, the model applied is not expected to be exactly true since it is not based on an underlying bio-physical model. However, the multiplicative model has so far been found to fit the data adequately, i.e. no significant deviations are detected for many common diseases for which many risk variants have been discovered.

As an example, an individual who has the following genotypes at 4 hypothetical markers associated with a particular condition (such as, e.g., abdominal aortic aneurysm, myocardial infarction, peripheral arterial disease and/or venous thromboembolism) along with the risk relative to the population at each marker:

| Marker | Genotype | Calculated risk |
|---|---|---|
| M1 | CC | 1.03 |
| M2 | GG | 1.30 |
| M3 | AG | 0.88 |
| M4 | TT | 1.54 |

Combined, the overall risk relative to the population for this individual is: 1.03×1.30×0.88×1.54=1.81.

### Adjusted Life-Time Risk

The lifetime risk of an individual is derived by multiplying the overall genetic risk relative to the population with the average life-time risk of the disease in the general population of the same ethnicity and gender and in the region of the individual's geographical origin. As there are usually several epidemiologic studies to choose from when defining the general population risk, we will pick studies that are well-powered for the disease definition that has been used for the genetic variants.

For example, if the overall genetic risk relative to the population is 1.8 for an individual for a particular disease, and if the average life-time risk of the disease for individuals of his demographic is 20%, then the adjusted lifetime risk for the individual is 20%×1.8=36%.

Note that since the average RR for a population is one, this multiplication model provides the same average adjusted life-time risk of the disease. Furthermore, since the actual life-time risk cannot exceed 100%, there must be an upper limit to the genetic RR.

### Determining Risk

In the present context, an individual who is at an increased susceptibility (i.e., increased risk) for a condition selected from the group consisting of abdominal aortic aneurysm, myocardial infarction, peripheral arterial disease and venous thromboembolism, is an individual in whom at least one specific allele at one or more polymorphic marker conferring increased susceptibility (increased risk) for the condition is identified (i.e., at-risk marker alleles). The at-risk marker, or an at-risk haplotype, is one that confers an increased risk (increased susceptibility) of the condition, i.e. particular allele(s) at the at-risk marker confer increased risk of the condition. Individuals who are homozygous for at-risk alleles at such markers are at particularly high risk. In one embodiment, significance associated with a marker or haplotype is measured by a relative risk (RR). In another embodiment, significance associated with a marker or haplotye is measured by an odds ratio (OR). In a further embodiment, the significance is measured by a percentage. In one embodiment, a significant increased risk is measured as a risk (relative risk and/or odds ratio) of at least 1.10, including but not limited to: at least 1.15, at least 1.16, at least 1.17, at least 1.18, at least 1.19, at least 1.20, at least 1.21, at least 1.25, at least 1.30, at least 1.35, and at least 1.40. In a particular embodiment, a risk (relative risk and/or odds ratio) of at least 1.20 is significant. In another particular embodiment, a risk of at least 1.21 is significant. In yet another embodiment, a risk of at least 1.30 is significant. In a further embodiment, a relative risk of at least 1.40 is significant. In another further embodiment, a significant increase in risk is at least 1.45 is significant. In other embodiments, a significant increase in risk is at least about 10%, including but not limited to about 11%, 12%, 13%, 14%, 15%, 16%, 17%, 18%, 19%, 20%, 21%, 22%, 23%, 24%, 25%, 30%, 35%, 40%, and 45%. In one particular embodiment, a significant increase in risk is at least 20%. In other embodiments, a significant increase in risk is at least 21%, at least 25%, at least 30%, or at least 40%. Other cutoffs or ranges as deemed suitable by the person skilled in the art to characterize the invention are however also contemplated, and those are also within scope of the present invention. In certain embodiments, a significant increase in risk is characterized by a p-value, such as a p-value of less than 0.05, less than 0.01, less than 0.001, less than 0.0001, less than 0.00001, less than 0.000001, less than 0.0000001, less than 0.00000001, or less than 0.000000001.

An at-risk polymorphic marker or haplotype as described herein is one where at least one allele of at least one marker or haplotype is more frequently present in an individual at risk for the disease (or trait) (affected), or diagnosed with the disease, compared to the frequency of its presence in a comparison group (control), such that the presence of the marker or haplotype is indicative of susceptibility to the disease. The control group may in one embodiment be a population sample, i.e. a random sample from the general population. In another embodiment, the control group is represented by a group of individuals who are disease-free. Such disease-free controls may in one embodiment be characterized by the absence of one or more specific disease-associated symptoms. Alternatively, the disease-free controls are those that have not been diagnosed with the disease. In another embodiment, the disease-free control group is characterized by the absence of one or more disease-specific risk factors. Such risk factors are in one embodiment at least one environmental risk factor. Representative environmental factors are natural products, minerals or other chemicals which are known to affect, or contemplated to affect, the risk of developing the specific disease. In one embodiment, the risk factors comprise at least one additional genetic risk factor.

The person skilled in the art will appreciate that for markers with two alleles present in the population being studied (such as SNPs), and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

Thus, in other embodiments of the invention, an individual who is at a decreased susceptibility (i.e., at a decreased risk) for a condition selected from the group consisting of abdominal aortic aneurysm, myocardial infarction, peripheral arterial disease and venous thromboembolism is an individual in whom at least one specific allele at one or more polymorphic marker conferring decreased susceptibility for the condition is identified. Alternatively, an individual who is at decreased susceptibility for the condition is an individual in whom the absence of at least one specific allele conferring increased susceptibility for the condition is identified. The marker alleles and/or haplotypes conferring decreased risk are also said to be protective. In one aspect, the protective marker or haplotype is one that confers a significant decreased risk (or susceptibility) of the condition. In one embodiment, significant decreased risk is measured as a relative risk (or odds ratio) of less than 0.9, including but not limited to less than 0.89, less than 0.88, less than 0.87, less than 0.86, less than 0.85, less than 0.84, less than 0.83, less than 0.82 and less than 0.80. In one particular embodiment, significant decreased risk is less than 0.85. In another embodiment, significant decreased risk is less than 0.83. In another embodiment, the decrease in risk (or susceptibility) is at least 10%, including but not limited to at least 11%, 12%, 13%, 14%, 15%, 16%, 17%, 18%, 19%, 20%, 21%, 22%, 23%, 24%, 25%, 30%, 35%, 40%, and 45%. In one particular embodiment, a significant decrease in risk is at least about 20%. In another embodiment, a significant decrease in risk is at least about 21%. Individuals who are homozygous for protective variants (protective alleles or haplotypes) are at particularly decreased risk of the condition. For SNPs, individuals who are homozygous for the non-risk allele of the SNP are at particularly low risk of the condition.

### Database

Determining susceptibility can alternatively or additionally comprise comparing nucleic acid sequence data and/or genotype data to a database containing correlation data between polymorphic markers and susceptibility to a condition selected from the group consisting of abdominal aortic aneurysm, myocardial infarction, peripheral arterial disease and venous thromboembolism. The database can be part of a computer-readable medium described herein.

In a specific aspect of the invention, the database comprises at least one measure of susceptibility to the condition for the polymorphic markers. For example, the database may comprise risk values associated with particular genotypes at such markers. The database may also comprise risk values associated with particular genotype combinations for multiple such markers.

In another specific aspect of the invention, the database comprises a look-up table containing at least one measure of susceptibility to the condition for the polymorphic markers.

### Further Steps

The methods disclosed herein can comprise additional steps which may occur before, after, or simultaneously with one of the aforementioned steps of the method of the invention. In a specific embodiment of the invention, the method of determining a susceptibility to a condition selected from the group consisting of abdominal aortic aneurysm, myocardial infarction, peripheral arterial disease and venous thromboembolism further comprises reporting the susceptibility to at least one entity selected from the group consisting of the individual, a guardian of the individual, a genetic service provider, a physician, a medical organization, and a medical insurer. The reporting may be accomplished by any of several means. For example, the reporting can comprise sending a written report on physical media or electronically or providing an oral report to at least one entity of the group, which written or oral report comprises the susceptibility. Alternatively, the reporting can comprise providing the at least one entity of the group with a login and password, which provides access to a report comprising the susceptibility posted on a password-protected computer system.

### Study Population

In a general sense, the methods, kits and other aspects of the invention described herein can be utilized from samples containing nucleic acid material (DNA or RNA) from any source and from any individual, or from genotype or sequence data derived from such samples. In preferred embodiments, the individual is a human individual. The individual can be an adult, child, or fetus. The nucleic acid source may be any sample comprising nucleic acid material, including biological samples, or a sample comprising nucleic acid material derived therefrom. The present invention also provides for assessing markers and/or haplotypes in individuals who are members of a target population. Such a target population is in one embodiment a population or group of individuals at risk of developing the condition, e.g. a condition selected from the group consisting of abdominal aortic aneurysm, myocardial infarction, peripheral arterial disease and venous thromboembolism, based on other genetic factors, biomarkers, biophysical parameters (e.g., weight, BMD, blood pressure), or general health and/or lifestyle parameters (e.g., history of the condition or related condition, previous diagnosis of condition, family history of the condition).

The invention provides for embodiments that include individuals from specific age subgroups, such as those over or under a particular age, such as age of greater than 40, greater than 45, or greater than age 50, 55, 60, 65, 70, 75, 80, or 85. Other embodiments of the invention pertain to other age groups, such as individuals aged less than 85, such as less than age 80, less than age 75, or less than age 70, 65, 60, 55, 50, 45, 40, 35, or age 30. Other embodiments relate to individuals with age at onset of the condition in a particular age range, for example individuals with age at onset greater than age 50, 55, 60, 65, 70, 75, 80, or 85, or individuals with age at onset less than age 70, 65, 60, 55, 50, 45, 40, 35, or age 30. In certain embodiments, age at onset is suitably different for males and females. For example, in certain embodiments, the invention relates to individuals at risk for, or individuals having experienced, myocardial infarction with an early onset. In certain embodiments, the invention relates to male individuals with age at onset of myocardial infarction of less than 50 years and female individuals with age at onset of myocardial infarction of less than 60 years. In certain embodiments, age at onset of myocardial infarction is the age at which the individual experiences his/her first infarct. Individuals who carry at least one copy of the at-risk variants for myocardial infarction described herein (e.g., allele A of marker rs7025486) are thus at increased risk of developing myocardial infarction with an early age at onset. For males, the A allele of rs7025486 is predictive of increased risk of myocardial infarction before age 50. For females, the A allele of rs7025486 is predictive of increased risk of myocardial infarction before age 60.

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Gudmundsson, J., et al. Nat Genet. 41:1122-6 (2009); Stacey, S, N., et al. Nat Genet. 41:909-14 (2009); Thorleifsson, G. et al. Nat Genet. 41:926-30 (2009); Sulem, P., et al. Nat Genet May 17, 2009 (Epub ahead of print); Rafnar, T., et al. Nat Genet. 41:221-7 (2009); Gretarsdottir, S., et al. Ann Neurol 64:402-9 (2008); Stacey, S, N., et al. Nat Genet. 40:1313-18 (2008); Gudbjartsson, D. F., et al. Nat Genet. 40:886-91 (2008); Styrkarsdottir, U., et al. N Engl J Med 358:2355-65 (2008); Thorgeirsson, T., et al. Nature 452:638-42 (2008); Gudmundsson, J., et al. Nat. Genet. 40:281-3 (2008); Stacey, S. N., et al., Nat. Genet. 39:865-69 (2007); Helgadottir, A., et al., Science 316:1491-93 (2007); Steinthorsdottir, V., et al., Nat. Genet. 39:770-75 (2007); Gudmundsson, J., et al., Nat. Genet. 39:631-37 (2007); Frayling, T M, Nature Reviews Genet. 8:657-662 (2007); Amundadottir, L. T., et al., Nat. Genet. 38:652-58 (2006); Grant, S. F., et al., Nat. Genet. 38:320-23 (2006)). Thus, genetic findings in the Icelandic population have in general been replicated in other populations, including populations from Africa and Asia.

It is thus believed that the markers described herein to be associated with risk of vascular conditions such as abdominal aortic aneurysm, myocardial infarction, peripheral arterial disease and venous thromboembolism will show similar association in other human populations. Particular embodiments comprising individual human populations are thus also contemplated and within the scope of the invention. Such embodiments relate to human subjects that are from one or more human population including, but not limited to, Caucasian populations, European populations, American populations, Eurasian populations, Asian populations, Central/South Asian populations, East Asian populations, Middle Eastern populations, African populations, Hispanic populations, and Oceanian populations. European populations include, but are not limited to, Swedish, Norwegian, Finnish, Russian, Danish, Icelandic, Irish, Kelt, English, Scottish, Dutch, Belgian, French, German, Spanish, Portuguese, Italian, Polish, Bulgarian, Slavic, Serbian, Bosnian, Czech, Greek and Turkish populations.

The racial contribution in individual subjects may also be determined by genetic analysis. Genetic analysis of ancestry may be carried out using unlinked microsatellite markers such as those set out in Smith et al. (Am J Hum Genet. 74, 1001-13 (2004)).

In certain embodiments, the invention relates to markers and/or haplotypes identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, have different population frequency in different populations, or are polymorphic in one population but not in another. The person skilled in the art will however apply the methods available and as thought herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population.

### Screening Methods

The invention also provides a method of screening candidate markers for assessing susceptibility to a condition selected from the group consisting of abdominal aortic aneurysm, myocardial infarction, peripheral arterial disease and venous thromboembolism. The invention also provides a method of identification of a marker for use in assessing susceptibility to a condition selected from the group consisting of abdominal aortic aneurysm, myocardial infarction, peripheral arterial disease and venous thromboembolism. The method may comprise analyzing the frequency of at least one allele of a polymorphic marker in a population of human individuals diagnosed with the condition, wherein a significant difference in frequency of the at least one allele in the population of human individuals diagnosed with the condition as compared to the frequency of the at least one allele in a control population of human individuals is indicative of the allele as a marker of the condition. In certain embodiments, the candidate marker is a marker in linkage disequilibrium with rs7025486.

In one embodiment, the method comprises (i) identifying at least one polymorphic marker in linkage disequilibrium, as determined by values of r² of greater than 0.2, with rs7025486; (ii) obtaining sequence information about the at least one polymorphic marker in a group of individuals diagnosed with the condition; and (iii) obtaining sequence information about the at least one polymorphic marker in a group of control individuals; wherein determination of a significant difference in frequency of at least one allele in the at least one polymorphism in individuals diagnosed with the condition as compared with the frequency of the at least one allele in the control group is indicative of the at least one polymorphism being useful for assessing susceptibility to the condition.

In one embodiment, an increase in frequency of the at least one allele in the at least one polymorphism in individuals diagnosed with the condition, as compared with the frequency of the at least one allele in the control group, is indicative of the at least one polymorphism being useful for assessing increased susceptibility to the condition, and wherein a decrease in frequency of the at least one allele in the at least one polymorphism in individuals diagnosed with the condition, as compared with the frequency of the at least one allele in the control group, is indicative of the at least one polymorphism being useful for assessing decreased susceptibility to, or protection against, the condition.

### Utility of Genetic Testing

The person skilled in the art will appreciate and understand that the variants described herein in general do not, by themselves, provide an absolute identification of individuals who will develop a particular vascular condition such as abdominal aortic aneurysm, myocardial infarction, peripheral arterial disease, venous thromboembolism and pulmonary embolism. The variants described herein do however indicate increased and/or decreased likelihood that individuals carrying the at-risk or protective variants of the invention will develop the condition. The present inventors have discovered that certain variants confer increase risk of developing certain vascular condition, as supported by the statistically significant results presented in the Exemplification herein. This information is extremely valuable in itself, as outlined in more detail in the below, as it can be used to, for example, initiate preventive measures at an early stage, perform regular physical exams to monitor the progress and/or appearance of symptoms, or to schedule exams at a regular interval to identify early symptoms, so as to be able to apply treatment at an early stage.

The knowledge about a genetic variant that confers a risk of developing a particular disease or condition offers the opportunity to apply a genetic test to distinguish between individuals with increased risk of developing the disease (i.e. carriers of the at-risk variant) and those with decreased risk of developing the disease (i.e. carriers of the protective variant). The core values of genetic testing, for individuals belonging to both of the above mentioned groups, are the possibilities of being able to diagnose a susceptibility or predisposition to a disease at an early stage and/or provide information to the clinician about prognosis/aggressiveness of disease in order to be able to apply the most appropriate treatment.

In general, individuals with a family history of vascular conditions and carriers of at-risk variants for such conditions may benefit from genetic testing since the knowledge of the presence of a genetic risk factor, or evidence for increased risk of being a carrier of one or more risk factors, may provide increased incentive for implementing a healthier lifestyle (e.g. lose weight, change diet, increase exercise, give up smoking, etc.).

The polymorphic markers of the present invention can be used alone or in combination, as well as in combination with other factors, including other genetic risk factors or biomarkers, for risk assessment of an individual for vascular conditions. Many factors known to affect the predisposition of an individual towards developing risk of developing such conditions are known to the person skilled in the art and can be utilized in such assessment. These include, but are not limited to, age, gender, smoking, physical activity, waist-to-hip circumference ratio, family history of Cardiovascular Disease or MI, previously diagnosed cardiovascular disease, obesity, hypertriglyceridemia, low HDL cholesterol, hypertension, elevated blood pressure, cholesterol levels, HDL cholesterol, LDL cholesterol, triglycerides, apolipoprotein AI and B levels, fibrinogen, ferritin, C-reactive protein and leukotriene levels. Methods known in the art can be used for such assessment, including multivariate analyses or logistic regression.

### Diagnostic Methods

The polymorphic markers associated with increased susceptibility to vascular conditions (e.g., abdominal aortic aneurysm, myocardial infarction, peripheral arterial disease, venous thromboembolism and pulmonary embolism) are useful in diagnostic methods. While methods of diagnosing such conditions are known in the art, the detection of one or more alleles of the specific polymorphic markers advantageously may be useful for detection of disease at its early stages and may also reduce the occurrence of mis-diagnosis. In this regard, the invention further provides methods of diagnosing vascular conditions comprising obtaining sequence data, e.g., nucleic acid sequence data, identifying at least one allele of at least one polymorphic marker of a specified group, in conjunction with carrying out one or more steps, e.g., clinical diagnostic steps, such as any of those described herein.

With regard to the method of diagnosing the vascular condition, the group of polymorphic markers in one embodiment consists of rs7025486, and markers in linkage disequilibrium therewith. In a particular embodiment, markers in linkage disequilibrium with rs7025486 are selected from the group consisting of rs584985, rs2777310, rs2797348, rs12352132, rs2777308, rs2797347, rs1003016, rs12553641, rs12554667, rs10760182, rs10818577, rs10818578, rs10818579, rs10818580, rs10985344, rs885150, rs10818583, rs7025486, rs10985349, rs10818589, rs10116069, rs2416834, rs878708, rs669128, rs2009828, rs2777320, rs4836858, rs7038469, rs2777319, rs7869336, rs2797349, rs2777311, rs62575880, s.123444035, s.123444070, rs1768732, s.123445547, s.123446322, rs7465724, s.123446681, s.123447265, s.123449587, rs12554639, s.123451318, s.123451324, s.123451615, s.123451617, rs10818576, s.123455512, rs62572789, rs12380555, rs10985347, s.123459543, rs885150, s.123461786, s.123462115, rs1984038, rs1984037, s.123469017, s.123472214, rs12000685, rs12000723, rs35661033, s.123479977, rs1571804, s.123591409, and rs10985475

The present invention pertains in some embodiments to methods of clinical applications of diagnosis, e.g., diagnosis performed by a medical professional. In other embodiments, the invention pertains to methods of diagnosis or methods of determination of a susceptibility performed by a layman. The layman can be the customer of a sequencing or genotyping service. The layman may also be a genotype or sequencing service provider, who performs analysis on a DNA sample from an individual, in order to provide service related to genetic risk factors for particular traits or diseases, based on the genotype status of the individual (i.e., the customer). Sequencing methods include for example those discussed in the above, but in general any suitabgle sequencing method may be used in the methods described and claimed herein. Recent technological advances in genotyping technologies, including high-throughput genotyping of SNP markers, such as Molecular Inversion Probe array technology (e.g., Affymetrix GeneChip), and BeadArray Technologies (e.g., Illumina GoldenGate and Infinium assays) have made it possible for individuals to have their own genome assessed for up to one million SNPs simultaneously, at relatively little cost. The resulting genotype information, which can be made available to the individual, can be compared to information about disease or trait risk associated with various SNPs, including information from public literature and scientific publications.

The diagnostic application of disease-associated alleles as described herein, can thus for example be performed by the individual, through analysis of his/her genotype data, by a health professional based on results of a clinical test, or by a third party, including the genotype or sequencing service provider. The third party may also be service provider who interprets genotype or sequence information from the customer to provide service related to specific genetic risk factors, including the genetic markers described herein. In other words, the diagnosis or determination of a susceptibility of genetic risk can be made by health professionals, genetic counselors, third parties providing genotyping service, third parties providing risk assessment service or by the layman (e.g., the individual), based on information about the genotype status of an individual and knowledge about the risk conferred by particular genetic risk factors (e.g., particular SNPs). In the present context, the term “diagnosing”, “diagnose a susceptibility” and “determine a susceptibility” is meant to refer to any available diagnostic method, including those mentioned above.

In certain embodiments, a sample containing genomic DNA from an individual is collected. Such sample can for example be a buccal swab, a saliva sample, a blood sample, or other suitable samples containing genomic DNA, as described further herein. In certain embodiments, the sample is obtained by non-invasive means (e.g., for obtaining a buccal sample, saliva sample, hair sample or skin sample). In certain embodiments, the sample is obtained by non-surgical means, i.e. in the absence of a surgical intervention on the individual that puts the individual at substantial health risk. Such embodiments may, in addition to non-invasive means also include obtaining sample by extracting a blood sample (e.g., a venous blood sample). The genomic DNA obtained from the individual is then analyzed using any common technique available to the skilled person, such as high-throughput array technologies. Results from such genotyping are stored in a convenient data storage unit, such as a data carrier, including computer databases, data storage disks, or by other convenient data storage means. In certain embodiments, the computer database is an object database, a relational database or a post-relational database. The genotype data is subsequently analyzed for the presence of certain variants known to be susceptibility variants for a particular human conditions, such as the genetic variants described herein. Genotype data can be retrieved from the data storage unit using any convenient data query method. Calculating risk conferred by a particular genotype for the individual can be based on comparing the genotype of the individual to previously determined risk (expressed as a relative risk (RR) or and odds ratio (OR), for example) for the genotype, for example for an heterozygous carrier of an at-risk variant for a particular disease or trait. The calculated risk for the individual can be the relative risk for a person, or for a specific genotype of a person, compared to the average population with matched gender and ethnicity. The average population risk can be expressed as a weighted average of the risks of different genotypes, using results from a reference population, and the appropriate calculations to calculate the risk of a genotype group relative to the population can then be performed. Alternatively, the risk for an individual is based on a comparison of particular genotypes, for example heterozygous carriers of an at-risk allele of a marker compared with non-carriers of the at-risk allele. Using the population average may in certain embodiments be more convenient, since it provides a measure which is easy to interpret for the user, i.e. a measure that gives the risk for the individual, based on his/her genotype, compared with the average in the population. The calculated risk estimated can be made available to the customer via a website, preferably a secure website.

In certain embodiments, a service provider will include in the provided service all of the steps of isolating genomic DNA from a sample provided by the customer, performing genotyping or sequencing of the isolated DNA, calculating genetic risk based on the genotype or sequence data, and report the risk to the customer. In some other embodiments, the service provider will include in the service the interpretation of genotype data for the individual, i.e., risk estimates for particular genetic variants based on the genotype data for the individual. In some other embodiments, the service provider may include service that includes genotyping service and interpretation of the genotype data, starting from a sample of isolated DNA from the individual.

Decreased susceptibility is in general determined based on the absence of particular at-risk alleles and/or the presence of protective alleles. As discussed in more detail herein, for biallelic markers such as SNPs, the alternate allele of an at-risk allele is, by definition, a protective allele. Determinations of its presence, in particular for homozygous individuals, is thus indicative of a decreased susceptibility.

In one embodiment, determination of a susceptibility to a vascular condition can be accomplished using hybridization methods. (see Current Protocols in Molecular Biology, Ausubel, F. et al., eds., John Wiley & Sons, including all supplements). The presence of a specific marker allele can be indicated by sequence-specific hybridization of a nucleic acid probe specific for the particular allele. The presence of more than one specific marker allele or a specific haplotype can be indicated by using several sequence-specific nucleic acid probes, each being specific for a particular allele. A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample. The invention can also be reduced to practice using any convenient genotyping method, including commercially available technologies and methods for genotyping particular polymorphic markers.

To determine a susceptibility, a hybridization sample can be formed by contacting the test sample, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length that is sufficient to specifically hybridize under stringent conditions to appropriate mRNA or genomic DNA. In certain embodiments, the oligonucleotide is from about 15 to about 100 nucleotides in length. In certain other embodiments, the oligonucleotide is from about 20 to about 50 nucleotides in length. The nucleic acid probe can comprise all or a portion of the nucleotide sequence of any one of the sequences set forth in SEQ ID NO:1-77 described herein, or the probe can be the complementary sequence of such a sequence. Other suitable probes for use in diagnostic assays of the invention are described herein. Hybridization can be performed by methods well known to the person skilled in the art (see, e.g., Current Protocols in Molecular Biology, Ausubel, F. et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe. The process can be repeated for any markers of the present invention, or markers that make up a haplotype of the present invention, or multiple probes can be used concurrently to detect more than one marker alleles at a time.

Alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P., et al., Bioconjug. Chem. 5:3-7 (1994)). The PNA probe can be designed to specifically hybridize to a molecule in a sample suspected of containing one or more of the marker alleles that are associated with a vascular condition, as described herein.

In one embodiment of the invention, a test sample containing genomic DNA obtained from the subject is collected and the polymerase chain reaction (PCR) is used to amplify a fragment comprising one ore more markers or haplotypes of the present invention. As described herein, identification of a particular marker allele or haplotype can be accomplished using a variety of methods (e.g., sequence analysis, analysis by restriction digestion, specific hybridization, single stranded conformation polymorphism assays (SSCP), electrophoretic analysis, etc.). In another embodiment, diagnosis is accomplished by expression analysis, for example by using quantitative PCR (kinetic thermal cycling). This technique can, for example, utilize commercially available technologies, such as TaqMan® (Applied Biosystems, Foster City, Calif.). The technique can assess the presence of an alteration in the expression or composition of a polypeptide or splicing variant(s). Further, the expression of the variant(s) can be quantified as physically or functionally different.

In another embodiment of the methods of the invention, analysis by restriction digestion can be used to detect a particular allele if the allele results in the creation or elimination of a restriction site relative to a reference sequence. Restriction fragment length polymorphism (RFLP) analysis can be conducted, e.g., as described in Current Protocols in Molecular Biology, supra. The digestion pattern of the relevant DNA fragment indicates the presence or absence of the particular allele in the sample.

Determination of a susceptibility may also be made by examining expression and/or composition of a polypeptide encoded by a nucleic acid in those instances where the genetic marker(s) result in a change in the composition or expression of the polypeptide. In one embodiment, determination of a susceptibility is performed by examining expression and/or composition (e.g., amino acid sequence) of a human DAB2IP protein. It is well known that regulatory element affecting gene expression may be located far away, even as far as tenths or hundreds of kilobases away, from the promoter region of a gene. By assaying for risk variants of vascular conditions as described herein, it is thus possible to assess the expression level of a nearby gene, such as DAB2IP. Possible mechanisms affecting such genes include, e.g., effects on transcription, effects on RNA splicing, alterations in relative amounts of alternative splice forms of mRNA, effects on RNA stability, effects on transport from the nucleus to cytoplasm, and effects on the efficiency and accuracy of translation.

A variety of methods can be used for detecting protein expression levels, including enzyme linked immunosorbent assays (ELISA), Western blots, immunoprecipitations and immunofluorescence. A test sample from a subject is assessed for the presence of an alteration in the expression and/or an alteration in composition of the polypeptide encoded by a particular nucleic acid. An alteration in expression of a polypeptide encoded by the nucleic acid can be, for example, an alteration in the quantitative polypeptide expression (i.e., the amount of polypeptide produced). An alteration in the composition of a polypeptide encoded by the nucleic acid may be an alteration in the sequence of the polypeptide. In one embodiment, diagnosis of a susceptibility to a vascular condition is made by detecting a particular splicing variant of the a human DAB2IP gene, or a particular pattern of splicing variants.

In one embodiment, an antibody (e.g., an antibody with a detectable label) that is capable of specific binding to a polypeptide (e.g., a DAB2IP polypeptide) can be used. Antibodies can be polyclonal or monoclonal. An intact antibody, or a fragment thereof (e.g., Fv, Fab, Fab′, F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a labeled secondary antibody (e.g., a fluorescently-labeled secondary antibody) and end-labeling of a DNA probe with biotin such that it can be detected with fluorescently-labeled streptavidin.

### Prognostic Methods

In addition to the utilities described above, the polymorphic markers of the invention are useful in determining a prognosis of a human individual experiencing symptoms associated with, or an individual diagnosed with a vascular condition such as abdominal aortic aneurysm, myocardial infarction, peripheral arterial disease and venous thromboembolism. Accordingly, the invention provides a method of predicting prognosis of an individual experiencing symptoms associated with, or an individual diagnosed with, the condition. The method comprises obtaining sequence data about a human individual identifying at least one allele of at least one polymorphic marker associated with the condition, wherein different alleles of the at least one polymorphic marker are associated with different susceptibilities to the condition in humans, and predicting prognosis of the individual from the sequence data. In one embodiment, the at least one polymorphic marker selected from the group consisting of rs7025486, and markers in linkage disequilibrium therewith.

The prognosis predicted by the methods of the invention can be any type of prognosis relating to the progression of the condition, and/or relating to the chance of recovering from the condition. The prognosis can, for instance, relate to the severity of the condition, when the condition may take place (e.g., the likelihood of a myocardial infarction), or how the condition will respond to therapeutic treatment.

With regard to the prognostic methods described herein, the sequence data can be nucleic acid sequence data or amino acid sequence data. Suitable methods of obtaining each are known in the art, some of which are described herein.

### Methods for Predicting Response to Therapeutic Agents

As is known in the art, individuals can have differential responses to a particular therapy (e.g., a therapeutic agent or therapeutic method). Pharmacogenomics addresses the issue of how genetic variations (e.g., the variants (markers and/or haplotypes) of the invention) affect drug response, due to altered drug disposition and/or abnormal or altered action of the drug. Thus, the basis of the differential response may be genetically determined in part. Clinical outcomes due to genetic variations affecting drug response may result in toxicity of the drug in certain individuals (e.g., carriers or non-carriers of the genetic variants of the invention), or therapeutic failure of the drug. Therefore, the variants of the invention may determine the manner in which a therapeutic agent and/or method acts on the body, or the way in which the body metabolizes the therapeutic agent.

Accordingly, in one embodiment, the presence of a particular allele at a polymorphic site is indicative of a different response, e.g. a different response rate, to a particular treatment modality. This means that a patient diagnosed with a vascular condition such as abdominal aortic aneurysm, myocardial infarction, peripheral arterial disease and venous thromboembolism and carrying a certain allele at a polymorphic marker as described herein (e.g., an at-risk marker of the invention) would respond better to, or worse to, a specific therapeutic, drug and/or other therapy used to treat the condition. Therefore, the presence or absence of the marker allele could aid in deciding what treatment should be used for the patient. If the patient is positive for a marker allele, then the physician recommends one particular therapy, while if the patient is negative for the at least one allele of a marker, then a different course of therapy may be recommended (which may include recommending that no immediate therapy, other than serial monitoring for progression of symptoms, be performed). Thus, the patient's carrier status could be used to help determine whether a particular treatment modality should be administered. The value lies within the possibilities of being able to diagnose a disease or condition at an early stage, to select the most appropriate treatment, and provide information to the clinician about prognosis/aggressiveness of the condition in order to be able to apply the most appropriate treatment.

Another aspect of the invention relates to methods of selecting individuals suitable for a particular treatment modality, based on the their likelihood of developing particular complications or side effects of the particular treatment. It is well known that most therapeutic agents can lead to certain unwanted complications or side effects. Likewise, certain therapeutic procedures or operations may have complications associated with them. Complications or side effects of these particular treatments or associated with specific therapeutic agents can, just as diseases do, have a genetic component. It is therefore contemplated that selection of the appropriate treatment or therapeutic agent can in part be performed by determining the genotype of an individual, and using the genotype status of the individual to decide on a suitable therapeutic procedure or on a suitable therapeutic agent to treat the particular disease or condition. It is therefore contemplated that the polymorphic markers of the invention can be used in this manner. Indiscriminate use of a such therapeutic agents or treatment modalities may lead to unnecessary and needless adverse complications.

In view of the foregoing, the invention provides a method of assessing an individual for probability of response to a therapeutic agent for preventing, treating, and/or ameliorating symptoms associated with condition selected from the group consisting of abdominal aortic aneurysm, myocardial infarction, peripheral arterial disease and venous thromboembolism. In one embodiment, the method comprises: analyzing nucleic acid sequence data from a human individual for at least one polymorphic marker selected from the group consisting of rs7025486, and markers in linkage disequilibrium therewith, wherein determination of the presence of at least one allele of the at least one marker is indicative of a probability of a positive response to the therapeutic agent.

In a further aspect, the markers of the invention can be used to increase power and effectiveness of clinical trials. Thus, individuals who are carriers of at least one at-risk variant of the present invention may be more likely to respond to a particular treatment modality. In one embodiment, individuals who carry at-risk variants for a gene in a pathway and/or metabolic network for which a particular treatment (e.g., small molecule drug) is targeting (e.g, the DAB2IP gene), are more likely to be responders to the treatment. For some treatments, the genetic risk will correlate with less responsiveness to therapy. In another embodiment, individuals who carry at-risk variants for a gene, which expression and/or function is altered by the at-risk variant, are more likely to be responders (or non-responders) to a treatment modality targeting that gene, its expression or its gene product. This application can improve the safety of clinical trials, but can also enhance the chance that a clinical trial will demonstrate statistically significant efficacy, which may be limited to a certain sub-group of the population. Thus, one possible outcome of such a trial is that carriers of certain genetic variants, e.g., at-risk markers of the invention, are statistically significantly likely to show positive response to the therapeutic agent, i.e. experience alleviation of symptoms associated with a condition selected from the group consisting of abdominal aortic aneurysm, myocardial infarction, peripheral arterial disease and venous thromboembolism when taking the therapeutic agent or drug as prescribed. Another possible outcome is that genetic carriers show less favorable response to the therapeutic agent, or show differential side-effects to the therapeutic agent compared to the non-carrier. An aspect of the invention is directed to screening for such pharmacogenetic correlations.

### Kits

Kits useful in the methods of the invention comprise components useful in any of the methods described herein, including for example, primers for nucleic acid amplification, hybridization probes, restriction enzymes (e.g., for RFLP analysis), allele-specific oligonucleotides, antibodies that bind to an altered polypeptide encoded by a nucleic acid of the invention as described herein (e.g., a genomic segment comprising at least one polymorphic marker and/or haplotype of the present invention) or to a non-altered (native) polypeptide encoded by a nucleic acid of the invention as described herein, means for amplification of nucleic acids, means for analyzing the nucleic acid sequence of nucleic acids, means for analyzing the amino acid sequence of a polynucleotides, etc. The kits can for example include necessary buffers, nucleic acid primers for amplifying nucleic acids (e.g., a nucleic acid segment comprising one or more of the polymorphic markers as described herein), and reagents for allele-specific detection of the fragments amplified using such primers and necessary enzymes (e.g., dna polymerase). Additionally, kits can provide reagents for assays to be used in combination with the methods of the present invention, e.g., reagents for use with other diagnostic assays for vascular diseases such as abdominal aortic aneurysm, myocardial infarction, peripheral arterial disease and venous thromboembolism.

In one embodiment, the invention pertains to a kit for assaying a sample from a subject to detect a susceptibility to a vascular condition selected from the group consisting of abdominal aortic aneurysm, myocardial infarction, peripheral arterial disease and venous thromboembolism in the subject, wherein the kit comprises reagents necessary for selectively detecting at least one allele of at least one polymorphism of the present invention in the genome of the individual. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism associated with the condition risk. In one such embodiment, the polymorphism is selected from the group consisting of the polymorphisms rs7025486, and polymorphic markers in linkage disequilibrium therewith. In yet another embodiment the fragment is at least 20 base pairs in size. Such oligonucleotides or nucleic acids (e.g., oligonucleotide primers) can be designed using portions of the nucleic acid sequence flanking polymorphisms (e.g., SNPs or microsatellites) that are associated with risk of the vascular condition. In another embodiment, the kit comprises one or more labeled nucleic acids capable of allele-specific detection of one or more specific polymorphic markers or haplotypes, and reagents for detection of the label. Suitable labels include, e.g., a radioisotope, a fluorescent label, an enzyme label, an enzyme co-factor label, a magnetic label, a spin label, an epitope label.

In particular embodiments, the polymorphic marker(s) to be detected by the reagents of the kit comprises one or more markers, two or more markers, three or more markers, four or more markers or five or more markers selected from the group consisting of the markers set forth in Table 1 herein. In another embodiment, the marker or haplotype to be detected comprises at least one marker from the group of markers in strong linkage disequilibrium, as defined by values of r² greater than 0.2, to rs7025486. In another embodiment, the marker or haplotype to be detected is rs7025486.

In a preferred embodiment, the DNA template containing a SNP polymorphism is amplified by Polymerase Chain Reaction (PCR) prior to detection, and primers for such amplification are included in the reagent kit. In such an embodiment, the amplified DNA serves as the template for the detection probe and the enhancer probe.

In one embodiment, the DNA template is amplified by means of Whole Genome Amplification (WGA) methods, prior to assessment for the presence of specific polymorphic markers as described herein. Standard methods well known to the skilled person for performing WGA may be utilized, and are within scope of the invention. In one such embodiment, reagents for performing WGA are included in the reagent kit.

In certain embodiments, determination of the presence of a particular marker allele is indicative of a susceptibility (increased susceptibility or decreased susceptibility) to the vascular condition. In another embodiment, determination of the presence of a marker allele is indicative of prognosis of the vascular condition. In another embodiment, the presence of the marker allele or haplotype is indicative of response to a therapeutic agent for the condition. In yet another embodiment, the presence of the marker allele or haplotype is indicative of progress of treatment of the condition.

In a further aspect of the present invention, a pharmaceutical pack (kit) is provided, the pack comprising a therapeutic agent and a set of instructions for administration of the therapeutic agent to humans diagnostically tested for one or more variants of the present invention, as disclosed herein. The therapeutic agent can be a small molecule drug, an antibody, a peptide, an antisense or RNAi molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention (e.g., an at-risk variant) is instructed to take a prescribed dose of the therapeutic agent. In another embodiment, an individual identified as a non-carrier of at least one variant of the present invention (e.g., an at-risk variant) is instructed to take a prescribed dose of the therapeutic agent.

In certain embodiments, the kit further comprises a set of instructions for using the reagents comprising the kit. In certain embodiments, the kit further comprises a collection of data comprising correlation data between the polymorphic markers assessed by the kit and susceptibility to the vascular condition.

### Antisense Agents

The nucleic acids and/or variants described herein, or nucleic acids comprising their complementary sequence, may be used as antisense constructs to control gene expression in cells, tissues or organs. The methodology associated with antisense techniques is well known to the skilled artisan, and is for example described and reviewed in AntisenseDrug Technology: Principles, Strategies, and Applications, Crooke, ed., Marcel Dekker Inc., New York (2001). In general, antisense agents (antisense oligonucleotides) are comprised of single stranded oligonucleotides (RNA or DNA) that are capable of binding to a complimentary nucleotide segment. By binding the appropriate target sequence, an RNA-RNA, DNA-DNA or RNA-DNA duplex is formed. The antisense oligonucleotides are complementary to the sense or coding strand of a gene. It is also possible to form a triple helix, where the antisense oligonucleotide binds to duplex DNA.

Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Layery et al., Curr. Opin. Drug Discov. Devel. 6:561-569 (2003), Stephens et al., Curr. Opin. Mol. Ther. 5:118-122 (2003), Kurreck, Eur. J. Biochem. 270:1628-44 (2003), Dias et al., Mol. Cancer. Ter. 1:347-55 (2002), Chen, Methods Mol. Med. 75:621-636 (2003), Wang et al., Curr. Cancer Drug Targets 1:177-96 (2001), and Bennett, Antisense Nucleic Acid Drug. Dev. 12:215-24 (2002).

In certain embodiments, the antisense agent is an oligonucleotide that is capable of binding to a particular nucleotide segment. In certain embodiments, the nucleotide segment comprises the human DAB2IP gene. In certain other embodiments, the antisense nucleotide is capable of binding to a nucleotide segment of as set forth in any one of SEQ ID NO:1-77. Antisense nucleotides can be from 5-400 nucleotides in length, including 5-200 nucleotides, 5-100 nucleotides, 10-50 nucleotides, and 10-30 nucleotides. In certain preferred embodiments, the antisense nucleotides is from 14-50 nucleotides in length, including 14-40 nucleotides and 14-30 nucleotides.

The variants described herein can also be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention (i.e. certain marker alleles and/or haplotypes) can be inhibited or blocked. In one embodiment, the antisense molecules are designed to specifically bind a particular allelic form (i.e., one or several variants (alleles and/or haplotypes)) of the target nucleic acid, thereby inhibiting translation of a product originating from this specific allele or haplotype, but which do not bind other or alternate variants at the specific polymorphic sites of the target nucleic acid molecule. As antisense molecules can be used to inactivate mRNA so as to inhibit gene expression, and thus protein expression, the molecules can be used for disease treatment. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants presented herein can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev. Genet. 8:173-184 (2007), Chen & Rajewsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Layery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., curr. Opin. Genet. Dev. 10:562-7 (2000), Bosher et al., Nat. Cell Biol. 2:E31-6 (2000), and Hunter, Curr. Biol. 9:R440-442 (1999).

A genetic defect leading to increased predisposition or risk for development of a disease or condition, such as vascular condition selected from the group consisting of abdominal aortic aneurysm, myocardial infarction, peripheral arterial disease and venous thromboembolism, or a defect causing the disease, may be corrected permanently by administering to a subject carrying the defect a nucleic acid fragment that incorporates a repair sequence that supplies the normal/wild-type nucleotide(s) at the site of the genetic defect. Such site-specific repair sequence may concompass an RNA/DNA oligonucleotide that operates to promote endogenous repair of a subject's genomic DNA. The administration of the repair sequence may be performed by an appropriate vehicle, such as a complex with polyethelenimine, encapsulated in anionic liposomes, a viral vector such as an adenovirus vector, or other pharmaceutical compositions suitable for promoting intracellular uptake of the adminstered nucleic acid. The genetic defect may then be overcome, since the chimeric oligonucleotides induce the incorporation of the normal sequence into the genome of the subject, leading to expression of the normal/wild-type gene product. The replacement is propagated, thus rendering a permanent repair and alleviation of the symptoms associated with the disease or condition.

### Nucleic Acids and Polypeptides

The nucleic acids and polypeptides described herein can be used in methods and kits of the present invention. An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20). Another example of an algorithm is BLAT (Kent, W. J. Genome Res. 12:656-64 (2002)).

Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988). In another embodiment, the percent identity between two amino acid sequences can be accomplished using the GAP program in the GCG software package (Accelrys, Cambridge, UK).

The present invention also provides isolated nucleic acid molecules that contain a fragment or portion that hybridizes under highly stringent conditions to a nucleic acid that comprises, or consists of, the nucleotide sequence of LD Block C09, or a nucleotide sequence comprising, or consisting of, the complement of the nucleotide sequence of LD Block C09, wherein the nucleotide sequence comprises at least one polymorphic allele contained in the markers and haplotypes described herein. In certain embodiments, the nucleic acids of the invention comprise or a portion of the nucleotide sequence of any one of the nucleotide sequences set forth in SEQ ID NO:1-77. The nucleic acid fragments of the invention are at least about 15, at least about 18, 20, 23 or 25 nucleotides, and can be up to 30, 40, 50, 100, 200, 300 or 400 nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques well known to the skilled person. The amplified DNA can be labeled (e.g., radiolabeled, fluorescently labeled) and used as a probe for screening a cDNA library derived from human cells. The cDNA can be derived from mRNA and contained in a suitable vector. Corresponding clones can be isolated, DNA obtained following in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced and further characterized.

### Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

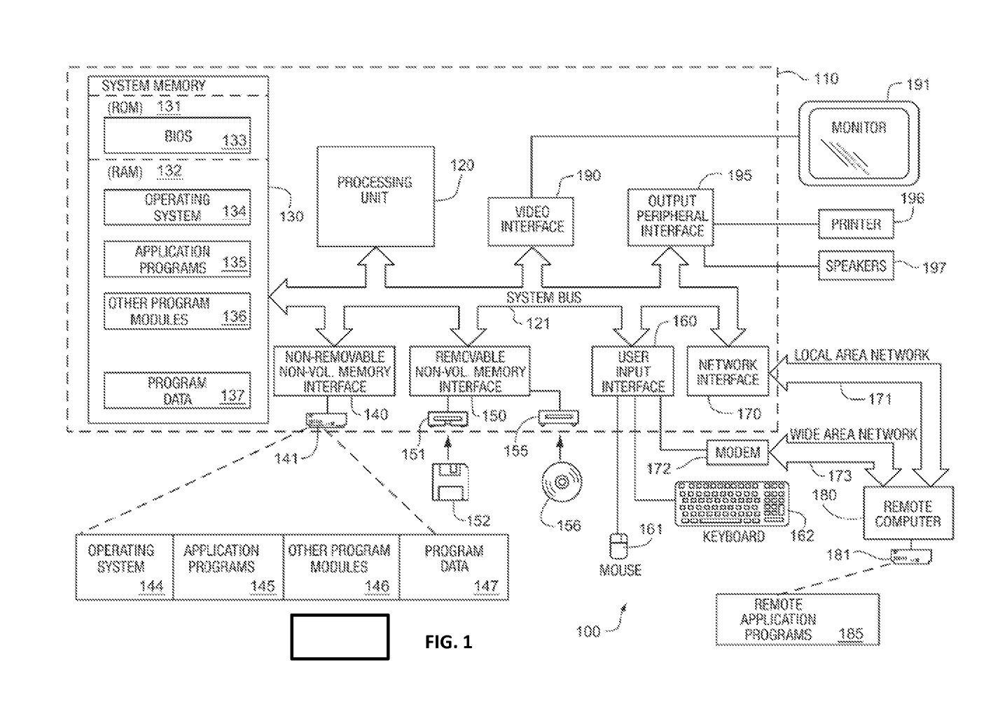

FIG. 1 illustrates an example of a suitable computing system environment 100 on which a system for the steps of the claimed method and apparatus may be implemented. The computing system environment 100 is only one example of a suitable computing environment and is not intended to suggest any limitation as to the scope of use or functionality of the method or apparatus of the claims. Neither should the computing environment 100 be interpreted as having any dependency or requirement relating to any one or combination of components illustrated in the exemplary operating environment 100.

The steps of the claimed method and system are operational with numerous other general purpose or special purpose computing system environments or configurations. Examples of well-known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the claimed method and system may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices.

With reference to FIG. 1, an exemplary system for implementing the steps of the claimed method and system includes a general purpose computing device in the form of a computer 110. Components of computer 110 may include, but are not limited to, a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (USA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 110 typically includes a variety of computer readable media. Computer readable media can be any available media that can be accessed by computer 110 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 1 illustrates operating system 134, application programs 135, other program modules 136, and program data 137.

The computer 110 may also include other removable/non-removable, volatile/nonvolatile computer storage media. By way of example only, FIG. 1 illustrates a hard disk drive 140 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 1, provide storage of computer readable instructions, data structures, program modules and other data for the computer 110. In FIG. 1, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a keyboard 162 and pointing device 161, commonly referred to as a mouse, trackball or touch pad. Other input devices (not shown) may include a microphone, joystick, game pad, satellite dish, scanner, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 1. The logical connections depicted in FIG. 1 include a local area network (LAN) 171 and a wide area network (WAN) 173, but may also include other networks. Such networking environments are commonplace in offices, enterprise-wide computer networks, intranets and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 1 illustrates remote application programs 185 as residing on memory device 181. It will be appreciated that the network connections shown are exemplary and other means of establishing a communications link between the computers may be used.

While the risk evaluation system and method, and other elements, have been described as preferably being implemented in software, they may be implemented in hardware, firmware, etc., and may be implemented by any other processor. Thus, the elements described herein may be implemented in a standard multi-purpose CPU or on specifically designed hardware or firmware such as an application-specific integrated circuit (ASIC) or other hard-wired device as desired, including, but not limited to, the computer 110 of FIG. 1. When implemented in software, the software routine may be stored in any computer readable memory such as on a magnetic disk, a laser disk, or other storage medium, in a RAM or ROM of a computer or processor, in any database, etc. Likewise, this software may be delivered to a user or a diagnostic system via any known or desired delivery method including, for example, on a computer readable disk or other transportable computer storage mechanism or over a communication channel such as a telephone line, the internet, wireless communication, etc. (which are viewed as being the same as or interchangeable with providing such software via a transportable storage medium).

Thus, many modifications and variations may be made in the techniques and structures described and illustrated herein without departing from the spirit and scope of the present invention. Thus, it should be understood that the methods and apparatus described herein are illustrative only and are not limiting upon the scope of the invention.

Accordingly, certain aspects of the invention relate to computer-implemented applications using the polymorphic markers and haplotypes described herein, and genotype and/or disease-association data derived therefrom. Such applications can be useful for storing, manipulating or otherwise analyzing genotype data that is useful in the methods of the invention. One example pertains to storing genotype information derived from an individual on readable media, so as to be able to provide the genotype information to a third party (e.g., the individual, a guardian of the individual, a health care provider or genetic analysis service provider), or for deriving information from the genotype data, e.g., by comparing the genotype data to information about genetic risk factors contributing to increased susceptibility to the disease, and reporting results based on such comparison.

In certain embodiments, computer-readable media suitably comprise capabilities of storing (i) identifier information for at least one polymorphic marker or a haplotype, as described herein; (ii) an indicator of the identity (e.g., presence or absence) of at least one allele of said at least one marker, or a haplotype, in individuals with the disease; and (iii) an indicator of the risk associated with the marker allele or haplotype.

The markers and haplotypes described herein to be associated with increased susceptibility (increased risk) of vascular conditions (e.g., abdominal aortic aneurysm, myocardial infarction, peripheral arterial disease and venous thromboembolism), are in certain embodiments useful for interpretation and/or analysis of genotype data. Thus in certain embodiments, determination of the presence of an at-risk allele for these conditions, as shown herein, or determination of the presence of an allele at a polymorphic marker in LD with any such risk allele, is indicative of the individual from whom the genotype data originates is at increased risk of the condition. In one such embodiment, genotype data is generated for at least one polymorphic marker shown herein to be associated with vascular conditions, or a marker in linkage disequilibrium therewith (e.g., rs7025486, and markers in linkage disequilibrium therewith). The genotype data may subsequently made available to a third party, such as the individual from whom the data originates, his/her guardian or representative, a physician or health care worker, genetic counsellor, or insurance agent, for example via a user interface accessible over the internet, together with an interpretation of the genotype data, e.g., in the form of a risk measure (such as an absolute risk (AR), risk ratio (RR) or odds ratio (OR)) for the disease. In another embodiment, at-risk markers identified in a genotype dataset derived from an individual are assessed and results from the assessment of the risk conferred by the presence of such at-risk variants in the dataset are made available to the third party, for example via a secure web interface, or by other communication means. The results of such risk assessment can be reported in numeric form (e.g., by risk values, such as absolute risk, relative risk, and/or an odds ratio, or by a percentage increase in risk compared with a reference), by graphical means, or by other means suitable to illustrate the risk to the individual from whom the genotype data is derived.

In certain embodiments, a report is prepared, which contains results of a determination of susceptibility of a vascular condition. The report may suitably be written in any computer readable medium, printed on paper, or displayed on a visual display.

The present invention will now be exemplified by the following non-limiting examples.

### Example 1

Abdominal aortic aneurysm (AAA), defined as an increase in the aortic diameter of 50% or infrarenal diameter of 30 mm¹, is a fairly common disease with a prevalence of up to 9% in men older than 65 years of age². The main risk factors for the development of AAA include advanced age, male gender, smoking, atherosclerosis and family history. This disorder is a significant public health problem, accounting for more than 150,000 hospital admissions, 40,000 repair operations³ and 15,000 deaths annually in the US². The mainstay of treatment is surveillance and surgery of aneurysms at high risk of rupture, judged primarily by size and growth rate⁴. Unfortunately, most AAA5 are asymptomatic until near-rupture or rupture, a catastrophic event with very high mortality⁵. In the US, screening by abdominal ultrasound is recommended for high risk individuals, including men aged 60 years or over with a positive family history and male smokers aged 65 to 75 years⁶.

There is a substantial heritable contribution to the development of AAA. A recent twin study showed an estimated 70% heritability⁷ and others have shown increased incidence of AAA in first degree relatives of affected individuals^{8,9}. Several studies have attempted to identify genetic risk variants. Linkage studies have reported two loci that map to chromosomes 19q13 and 4q31^{10,11}, however, predisposing sequence variants in these regions have not been identified. The majority of studies that have searched for AAA susceptibility variants have focused on candidate genes involved in extracellular matrix metabolism, inflammation and immune responses but no major risk gene has yet to be found through this approach⁹. Recently, a common sequence variant on chromosome 9p21 (rs10757278) near the CDKN2A and CDKN2B genes was found to associate with AAA (OR=1.31 and P=1.2×10⁻¹²)¹², an observation confirmed in independent studies^{13,14}. One AAA GWAS has been published and yielded a locus on 3p12.3, tagged by rs7635818 (OR=1.33 and P=0.0028)¹⁵. However, this finding has yet to be confirmed in independent samples.

### Methods

Association Analysis:

For case-control association analysis we utilized a standard likelihood ratio statistic, implemented in the NEMO software³⁶ to calculate two-sided P values and odds ratios (ORs) for each individual allele, assuming a multiplicative model for risk³⁷. The correlation between the risk variant and quantitative traits such as age of onset of MI, BMI, lipid level or smoking quantity was tested by regressing the trait on the number of copies of the risk allele an individual carries. We tested for epistatic interaction between rs7025486[A] and rs10757278[G] by correlating the number of risk alleles an individual carries in multiple regression adjusting for different sample sets by including corresponding indicator variables. This was done separately for the AAA cases and controls, excluding the Danish sample set for which rs10757278[G] was no genotyped. In all tables allelic frequencies are presented for the markers and all reported P values are two-sided.

Familial Imputation:

For the Icelandic dataset we extended the classical case-control association analysis to include in-silico genotypes of cases that are not genotyped but that have genotyped relatives³⁸ among the 40,000 Icelanders (about 13% of all living Icelanders) genotyped with the Illumina SNP chips at deCODE genetics. For every un-genotyped case we calculate the probability distribution of the genotypes of its relatives given its four possible phased genotypes. In practice we include only genotypes of the case's parents, children, siblings, half-siblings (and the half-sibling's parents), grand-parents, grand-children (and the grand-children's parents) and spouses. The contribution of the un-genotyped cases through this familial imputation to the effective sample size of the cases, n_{a,eff}, was estimated using the Fisher information.

Genomic Control:

Some of the individuals in the Icelandic case-control groups are related to each other, causing the χ²-test statistic to have a mean >1 and median >0.455. We estimated the genome-wide inflation factor λ_{g} as the average of the 293,677 χ²-statistics to adjust for both relatedness and potential population stratification.³⁹ This was done both for the primary AAA genome-wide analysis and for other traits tested in the Icelandic dataset where a genome-wide analysis was carried out to estimate the inflation factor. The P values presented for the Icelandic case-control groups in Tables 2-8 are adjusted using these inflation factors.

Analysis of the New Zealand Sample Set:

Only part of the AAA cases and controls from New Zealand (594 cases and 527 controls) were directly genotyped with single SNP genotyping. The rest, and about 54% of individuals typed with single SNP genotyping, were genotyped with the Affymetrix SNP 6.0 array and either direct or imputed genotypes were available for all the 19 variants. These two datasets were analysed together in the case-control analysis using the NEMO software. Genotypes not directly genotyped with single SNP genotyping or with the Affymetrix chip were treated as missing but the imputed genotypes were included in the analysis and used to provide partial information on the missing genotypes. To handle uncertainty with phase and missing genotypes, maximum likelihood estimates, likelihood ratios and P value are computed directly for the observed data, and hence the loss of information due to uncertainty in phase and missing genotypes is automatically captured by the likelihood ratios ³⁶.

Meta-Analysis:

Results from multiple case-control groups, both when combining the Icelandic and Dutch genome-wide analysis and when combining the follow-up sets, were combined using a Mantel-Haenszel model¹⁶ in which the groups were allowed to have different population frequencies for alleles and genotypes but were assumed to have common relative risks (a fixed effect model). Heterogeneity in the effect estimate was tested assuming that the estimated OR for different groups follow a log-normal distribution and using a likelihood ratio χ²-test with degrees of freedom equal to the number of groups compares minus one.

SNP Imputation:

Additional SNPs at the 9q33 loci, not genotyped on the Illumina SNP bead-chips, were imputed using the IMPUTE software⁴⁰ using the HapMap CEU dataset (v22)¹⁷ as training set. In all 520 SNPs in a 630 kb interval that includes DAB2IP and 200 kb up and downstream of the gene were imputed both for the Icelandic and the Dutch sample sets. For the Icelandic dataset the analysis was restricted to directly genotyped individuals to avoid complication in combining imputation of SNPs and imputation of un-genotyped individuals.

Sibling Relative Risk:

The contribution to the sibling relative risk λ_{sib} of AAA was calculated for both the 9p21 and 9q33 variant, assuming a multiplicative model for the risk, using the formula λ_{sib,i}=[1+pᵢ/(1−pᵢ)(βᵢ−1)²/(2((1−pᵢ)+βᵢpᵢ)²]² (ref 41), were βᵢ is the allelic odds ratio and pᵢ is the population frequency of variant i. For population frequency we used a simple average over study population in Table 4 and Table 7, respectively¹².

### Expression Analysis of DAB2IP

RNA was isolated from human whole blood, EBV transformed human lymphoblastoid cell lines, peripheral blood monocyte cells, human aortic smooth muscle cells (Sciencell, Cat. no. 6110) and human primary umbilical vein endothelial cells (HUVEC) using Qiagen RNA kits. Concentration and quality of the RNA was determined with Agilent 2100 Bioanalyzer (Agilent Technologies). cDNA were generated with High capacity cDNA reverse transcriptase kit (Applied Biosystems Inc.) and cDNA libraries for each tissue constructed by pooling the cDNA from several samples from each tissue. In addition to the libraries above, eleven commercial cDNA libraries (Clontech) were used. Real-time PCR assay was designed over the exon6-exon7 junction of the DAB2IP gene (RefSeq: NM_{—}032552.2). Left primer was GCCAAGACCAAGGAGGAGAT and right primer was GACATCATCAGGTCTGTCAGGA and Roche Universal Library probe was #37. The real-time PCR was run in duplicates for each cDNA tissue library according to recommendations on an ABI Prism 7900HT Sequence Detection System. Expression levels were normalized to the expression of DAB2IP in heart (User Bulletin no. 2, Applied Biosystems 2001).

In order to search for sequence variants that affect the risk of AAA we performed a GWAS using 452 Icelandic and 840 Dutch patients with AAA and 27,712 Icelandic and 2,791 Dutch controls, genotyped with the Illumina HumanHap370 or HumanHap610 SNP chips. Partial genotype information on an additional 536 Icelanders with AAA not typed with the SNP chips, but closely related to genotyped individuals, were also used in the analysis. We individually tested 293,677 SNPs that passed quality criteria, for association with AAA in the Icelandic and Dutch sample sets separately. The results for the Icelandic sample set were adjusted using the method of genomic control, dividing the χ² statistic by λ_{g}=1.143, while no adjustment was needed for the Dutch sample set (λ_{g}=1). The genome-wide association results for the two populations were combined assuming a fixed effect model¹⁶.

In the combined analysis of the two discovery sample sets, three correlated SNPs achieved genome-wide significance (P<1.6×10⁻⁷). These SNPs are all located at the previously discovered AAA associated CDKN2A/CDKN2B locus on 9p21. The associated odds ratios (OR) range from 1.25 to 1.27 (P=1.6×10⁻⁷ to 1.9×10⁻⁸; Table 2). In order to search for additional sequence variants associated with AAA we selected 22 SNPs with P<5.5×10⁻⁵ (excluding the 9p21 locus) for genotyping in four additional AAA sample sets of European ancestry from Belgium, Canada, New Zealand and the UK (follow-up set 1). The four sample sets combined included 1,665 patients with AAA and 1,931 controls. Nineteen of the 22 SNPs were successfully genotyped in the four sample sets. After combining the results for the two discovery sets and follow up set 1, one SNP, rs7025486[A] on 9q33, was associated with AAA at a genome-wide significance level (OR=1.24, P=1.8×10⁻⁹; Table 3). Additional genotypes were available for rs7025486 for 302

AAA cases from New Zealand that were included in follow-up set 1. For further validation we genotyped rs7025486 in four additional sample sets of European ancestry from Denmark, The Netherlands (Nijmegen) and two US populations from Danville and Pittsburgh (follow-up set 2) totalling 1,300 patients with AAA and 5,520 controls. The observed effect is weaker in set 2 compared to set 1, (OR=1.11 compared to 1.28, Table 4), however this difference is not significant (P=0.079). In the combined analysis of the discovery sets and follow-up sets 1 and 2, rs7025486[A] was associated with AAA with OR=1.21 and P=4.6×10⁻¹° (Table 4). No significant heterogeneity was observed in the effect estimates between the study populations (Pₕₑₜ=0.37). The 3p12.3 sequence variant rs7635818, previously identified by Elmore et al.¹⁵ does not associate with AAA in our two discovery sample sets (P=0.76). Analysis of 520 SNPs in a 630 Mb region centered on rs7025486[A] with genotypes imputed based on the CEU HapMap data¹⁷ did not yield additional SNPs that associate with AAA after adjusting for the number of tests done (data not shown).

To evaluate potential epistatic interaction between the new AAA variant, rs7025486[A] and the previously established AAA variant rs10757278[G] on 9p21, we tested their correlation within cases and controls in all our AAA case-control sample sets (except for the Danish sample set for which we did not have rs10757278[G] typed). The correlation was tested by regressing the number of copies of rs7025486[A] on the number of copies of rs10757278[G] an individual carries, adjusting for the different sample sets by including corresponding indicator variables in the analysis. In neither the case nor the control groups did we detect significant correlation (AAA cases P=0.21 and the controls P=0.53).

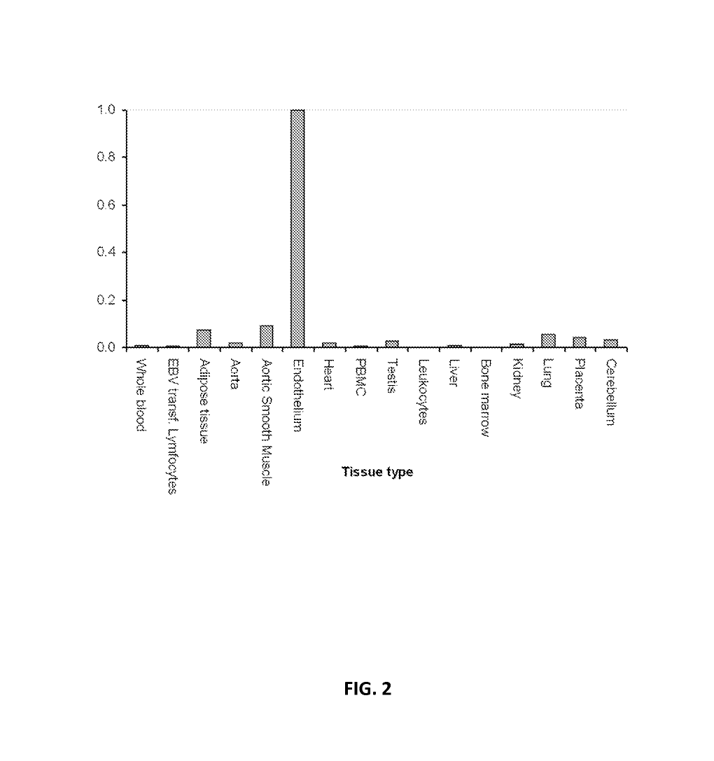

The sequence variant rs7025486[A] maps within intron 1 of the DAB2IP gene (DAB2 interacting protein) also called AIP1 (ASK1-interacting protein). DAB2IP is a member of the RAS-GTPase-activating protein family¹⁸. The DAB2IP protein has been shown to suppress cell survival and proliferation through suppression of the PI3K-Act and RAS pathways and to induce apoptosis through activation of ASK1, a member of the JNK and p38 MAPK pathways¹⁹. DAB2IP expression is often found to be down-regulated in human cancers^{20,21}, suggesting that it may function as a tumour suppressor gene. Based on gene expression databases DAB2IP is expressed in many tissues (http://biogps.gnf.org/#goto=genereport&id=153090) and our data further demonstrate DAB2IP expression in cardiovascular tissue such as aortic smooth muscle cells, heart and human umbilical vein endothelial cells (HUVEC) with highest expression by far in HUVEC (FIG. 2). The sequence variant rs7025486[A] showed nominally significant correlation with the level of DAB2IP expression in adipose tissue, mammary artery and ascending aorta however, the observed correlation was weak and the direction of the effect was not consistent between tissues (Table 5). Many studies have underscored the pivotal role of the PI3K-Akt signalling pathway in the vascular endothelium, affecting endothelial cell proliferation and survival as well as endothelial cell migration, and NO production^{22,23}. Furthermore, the JNK pathway has been implicated in the pathogenesis of AAA both in mice and in humans²⁴. Lastly, a recent study has also identified DAB2IP as an endogenous inhibitor of VEGFR2-mediated signalling²⁵, an important regulator of angiogenesis control.

The previously discovered AAA variant, rs10757278[G] at the CDKNA2/CDKNB2 locus on chromosome 9p21, was originally identified as a risk variant for MI and CAD in two GWAS^{26,27}. It was subsequently shown to affect the risk of two distinct aneurysmal diseases, AAA and intracranial aneurysm (IA), with comparable effects. The same variant also affects the risk of peripheral arterial disease (PAD) and large artery and cardiogenic (LAA/CE) stroke, but with less effect¹². In light of the broad vascular effect of the 9p21, AAA risk variant, we tested for association between rs7025486[A] at the DAB2IP locus and other vascular diseases. We tested for association with MI in seven sample sets of European ancestry (6,096 MI cases and 10,757 controls) (Table 6), and with PAD in seven European sample sets (3,690 PAD cases and 12,271 controls) (Table 7). We also included four sample sets of venous thromboembolism (VTE) (1,908 individuals with VTE, of which 811 had pulmonary embolism (PE), and 7,055 controls) as recent studies have pointed to a link between VTE and cardiovascular diseases²⁸′²⁹. For the Icelandic sample sets we used 5,863 Icelandic population controls without history of vascular diseases who were not in the control set of 27,712 individuals used for the AAA GWAS. The frequency of the risk variant in this control set is 0.292 and is not significantly different from the frequency of 0.298 in the control set used in the genome-wide analysis of AAA (P=0.29).

While rs7025486[A] has a modest effect on all MI (OR=1.09, P=0.0012), restricting the analysis to early-onset MI cases, defined as an event before age 50 years for men and 60 years for women, the risk is significantly increased (OR=1.18, P=3.1×10⁻⁵) even after adjusting for the number of vascular diseases tested. Regressing the age of onset of MI on the number of copies of rs7025486[A] an individual carries shows that each copy decreases the age of onset by approximately 0.48 years (P=0.034). This is about half the effect of the 9p21 variant rs10757278[G] where each copy corresponds to a decrease of about 1 year²⁶.

In addition to early-onset MI, rs7025486[A] associates with increased risk of PAD (OR=1.14, P=3.9×10⁻⁵) and PE (OR=1.20, P=0.00030), while the effect was weaker for VTE (OR=1.12, P=0.0079). Repeating the association analysis after excluding known cases of AAA, CAD or PAD from the group of VTE or PE cases did not change the observed effect (Table 8), indicating that the association with VTE/PE is not simply the consequence of the association between rs7025486[A] and the other cardiovascular diseases. The observed association of rs7025486[A] with VTE and PE prompted us to test the association of the 9p21 variant (rs10757278 [G]) with VTE and PE. For the four VTE and PE sample sets tested no association was observed (VTE, OR=1.05, P=0.17 and PE, OR=1.04, P=0.40).

### REFERENCES

1. Crawford, C. M., Hurtgen-Grace, K., Talarico, E. & Marley, J. Abdominal aortic aneurysm: an illustrated narrative review. J Manipulative Physiol Ther 26, 184-95 (2003).
2. Weintraub, N. L. Understanding abdominal aortic aneurysm. N Engl J Med 361, 1114-6 (2009).
3. Annambhotla, S. et al. Recent advances in molecular mechanisms of abdominal aortic aneurysm formation. World J Surg 32, 976-86 (2008).
4. Grootenboer, N., Bosch, J. L., Hendriks, J. M. & van Sambeek, M. R. Epidemiology, aetiology, risk of rupture and treatment of abdominal aortic aneurysms: does sex matter? Eur J Vasc Endovasc Surg 38, 278-84 (2009).
5. Assar, A. N. & Zarins, C. K. Ruptured abdominal aortic aneurysm: a surgical emergency with many clinical presentations. Postgrad Med J 85, 268-73 (2009).
6. Hirsch, A. T. et al. ACC/AHA 2005 Practice Guidelines for the management of patients with peripheral arterial disease (lower extremity, renal, mesenteric, and abdominal aortic): a collaborative report from the American Association for Vascular Surgery/Society for Vascular Surgery, Society for Cardiovascular Angiography and Interventions, Society for Vascular Medicine and Biology, Society of Interventional Radiology, and the ACC/AHA Task Force on Practice Guidelines (Writing Committee to Develop Guidelines for the Management of Patients With Peripheral Arterial Disease): endorsed by the American Association of Cardiovascular and Pulmonary Rehabilitation; National Heart, Lung, and Blood Institute; Society for Vascular Nursing; TransAtlantic Inter-Society Consensus; and Vascular Disease Foundation. Circulation 113, e463-654 (2006).
7. Wahlgren, C. M., Larsson, E., Magnusson, P. K., Hultgren, R. & Swedenborg, J. Genetic and environmental contributions to abdominal aortic aneurysm development in a twin population. J Vasc Surg 51, 3-7; discussion 7 (2010).
8. Ogata, T. et al. The lifetime prevalence of abdominal aortic aneurysms among siblings of aneurysm patients is eightfold higher than among siblings of spouses: an analysis of 187 aneurysm families in Nova Scotia, Canada. J Vasc Surg 42, 891-7 (2005).
9. Sandford, R. M., Bown, M. J., London, N. J. & Sayers, R. D. The genetic basis of abdominal aortic aneurysms: a review. Eur J Vasc Endovasc Surg 33, 381-90 (2007).
10. Shibamura, H. et al. Genome scan for familial abdominal aortic aneurysm using sex and family history as covariates suggests genetic heterogeneity and identifies linkage to chromosome 19q13. Circulation 109, 2103-8 (2004).
11. Van Vlijmen-Van Keulen, C. J., Rauwerda, J. A. & Pals, G. Genome-wide linkage in three Dutch families maps a locus for abdominal aortic aneurysms to chromosome 19q13.3. Eur J Vasc Endovasc Surg 30, 29-35 (2005).
12. Helgadottir, A. et al. The same sequence variant on 9p21 associates with myocardial infarction, abdominal aortic aneurysm and intracranial aneurysm. Nat Genet. 40, 217-24 (2008).
13. Thompson, A. R. et al. Sequence variant on 9p21 is associated with the presence of abdominal aortic aneurysm disease but does not have an impact on aneurysmal expansion. Eur J Hum Genet. 17, 391-4 (2009).
14. Bown, M. J. et al. Association between the coronary artery disease risk locus on chromosome 9p21.3 and abdominal aortic aneurysm. Circ Cardiovasc Genet. 1, 39-42 (2008).
15. Elmore, J. R. et al. Identification of a genetic variant associated with abdominal aortic aneurysms on chromosome 3p12.3 by genome wide association. J Vasc Surg 49, 1525-31 (2009).
16. Mantel, N. & Haenszel, W. Statistical aspects of the analysis of data from retrospective studies of disease. J Natl Cancer Inst 22, 719-48 (1959).
17. Evans, D. M., Frazer, I. H. & Martin, N. G. Genetic and environmental causes of variation in basal levels of blood cells. Twin Res 2, 250-7 (1999).
18. Iwashita, S. & Song, S. Y. RasGAPs: a crucial regulator of extracellular stimuli for homeostasis of cellular functions. Mol Biosyst 4, 213-22 (2008).
19. Xie, D. et al. DAB2IP coordinates both PI3K-Akt and ASK1 pathways for cell survival and apoptosis. Proc Natl Acad Sci USA 107:2485-90 (2009).
20. Chen, H., Pong, R. C., Wang, Z. & Hsieh, J. T. Differential regulation of the human gene DAB2IP in normal and malignant prostatic epithelia: cloning and characterization. Genomics 79, 573-81 (2002).
21. Qiu, G. H. et al. Differential expression of hDAB2IPA and hDAB2IPB in normal tissues and promoter methylation of hDAB2IPA in hepatocellular carcinoma. J Hepatol 46, 655-63 (2007).
22. Dimmeler, S. et al. Activation of nitric oxide synthase in endothelial cells by Akt-dependent phosphorylation. Nature 399, 601-5 (1999).
23. Shiojima, I. & Walsh, K. Role of Akt signaling in vascular homeostasis and angiogenesis. Circ Res 90, 1243-50 (2002).
24. Yoshimura, K. et al. Regression of abdominal aortic aneurysm by inhibition of c-Jun N-terminal kinase. Nat Med 11, 1330-8 (2005).
25. Zhang, H. et al. AIP1 functions as an endogenous inhibitor of VEGFR2-mediated signaling and inflammatory angiogenesis in mice. J Clin Invest 118, 3904-16 (2008).
26. Helgadottir, A. et al. A common variant on chromosome 9p21 affects the risk of myocardial infarction. Science 316, 1491-3 (2007).
27. McPherson, R. et al. A common allele on chromosome 9 associated with coronary heart disease. Science 316, 1488-91 (2007).
28. Prandoni, P. Links between arterial and venous disease. J Intern Med 262, 341-50 (2007).
29. Sorensen, H. T., Horvath-Puho, E., Pedersen, L., Baron, J. A. & Prandoni, P. Venous thromboembolism and subsequent hospitalisation due to acute arterial cardiovascular events: a 20-year cohort study. Lancet 370, 1773-9 (2007).
30. Braekkan, S. K. et al. Family history of myocardial infarction is an independent risk factor for venous thromboembolism: the Tromso study. J Thromb Haemost 6, 1851-7 (2008).
31. Prandoni, P. et al. An association between atherosclerosis and venous thrombosis. N Engl Med 348, 1435-41 (2003).
32. Reich, L. M. et al. Prospective study of subclinical atherosclerosis as a risk factor for venous thromboembolism. J Thromb Haemost 4, 1909-13 (2006).
33. van der Hagen, P. B. et al. Subclinical atherosclerosis and the risk of future venous thrombosis in the Cardiovascular Health Study. J Thromb Haemost 4, 1903-8 (2006).
34. Glynn, R. J. et al. A randomized trial of rosuvastatin in the prevention of venous thromboembolism. N Engl J Med 360, 1851-61 (2009).
35. Morello, F., Perino, A. & Hirsch, E. Phosphoinositide 3-kinase signalling in the vascular system. Cardiovasc Res 82, 261-71 (2009).
36. Gretarsdottir, S. et al. The gene encoding phosphodiesterase 4D confers risk of ischemic stroke. Nat Genet. 35, 131-8 (2003).
37. Rice, J. A. Generalized likelihood ratio tests. in Mathematical Statistics and Data Analysis, Vol. 1 (ed. Rice, J. A.) 308-310 (International Thomson Publishing, 1995).
38. Rafnar, T. et al. Sequence variants at the TERT-CLPTM1L locus associate with many cancer types. Nat Genet. 41, 221-7 (2009).
39. Devlin, B. & Roeder, K. Genomic control for association studies. Biometrics 55, 997-1004 (1999).
40. Marchini, J., Howie, B., Myers, S., McVean, G. & Donnelly, P. A new multipoint method for genome-wide association studies by imputation of genotypes. Nat Genet. 39, 906-13 (2007).
41. Pall, G. S. et al. A novel transmembrane MSP-containing protein that plays a role in right ventricle development. Genomics 84, 1051-9 (2004).

TABLE 2
SNPs with P < 5.5 × 10⁻⁵ in the combined GWA analysis of the Icelandic and
Dutch AAA samples.
Listed are the twenty-five SNPs with P < 5.5 × 10⁻⁵ in the combined GWAS on AAA cases and
controls from Iceland and The Netherlands. For each SNP the table shows the chromosome and
position in NCBI Build 36, the allele tested for association (EA), the frequency in controls (f_{c}) and
cases (fₐ), the OR and P value for Iceland and The Netherlands separately and combined. The
three SNPs at 9p21 that reach genome-wide significance are indicated in bold. P values for the
Icelandic sample set have been adjusted using the method of genomic control.

| | | | | Iceland | | | | Netherland | | | | Combined | |
|---|---|---|---|---|---|---|---|---|---|---|---|---|---|
| Chr | SNP | Pos | EA | f_{c} | fₐ | OR | P | f_{c} | fₐ | OR | P | OR | P |
| 1 | rs487174 | 45422830 | T | 0.101 | 0.134 | 1.38 | 0.00059 | 0.094 | 0.116 | 1.26 | 0.016 | 1.32 | 3.3 × 10⁻⁵ |
| ″ | rs1998064 | 227322133 | A | 0.746 | 0.798 | 1.35 | 4.8 × 10⁻⁵ | 0.743 | 0.771 | 1.17 | 0.022 | 1.25 | 8.9 × 10⁻⁶ |
| 2 | rs355823 | 165905357 | T | 0.096 | 0.137 | 1.50 | 1.9 × 10⁻⁵ | 0.081 | 0.094 | 1.17 | 0.14 | 1.34 | 3.0 × 10⁻⁵ |
| 5 | rs1372319 | 77994012 | T | 0.652 | 0.693 | 1.21 | 0.0041 | 0.698 | 0.736 | 1.20 | 0.0044 | 1.21 | 5.3 × 10⁻⁵ |
| ″ | rs959461 | 169860675 | G | 0.204 | 0.250 | 1.31 | 0.00023 | 0.204 | 0.237 | 1.21 | 0.0065 | 1.26 | 6.2 × 10⁻⁶ |
| 6 | rs9268832 | 32498969 | T | 0.511 | 0.561 | 1.22 | 0.0011 | 0.424 | 0.460 | 1.16 | 0.013 | 1.19 | 5.3 × 10⁻⁵ |
| ″ | rs7761436 | 110548401 | G | 0.708 | 0.752 | 1.25 | 0.0015 | 0.658 | 0.694 | 1.18 | 0.0083 | 1.21 | 4.3 × 10⁻⁵ |
| ″ | rs783166 | 161086525 | T | 0.084 | 0.111 | 1.37 | 0.0020 | 0.107 | 0.138 | 1.34 | 0.0011 | 1.35 | 7.4 × 10⁻⁶ |
| ″ | rs1652500 | 161101477 | C | 0.154 | 0.193 | 1.32 | 0.00056 | 0.199 | 0.233 | 1.22 | 0.0056 | 1.26 | 1.3 × 10⁻⁵ |
| 7 | rs7798936 | 95025282 | A | 0.550 | 0.588 | 1.17 | 0.014 | 0.499 | 0.552 | 1.24 | 0.00034 | 1.20 | 1.7 × 10⁻⁵ |
| ″ | rs6979784 | 127470170 | G | 0.745 | 0.769 | 1.14 | 0.063 | 0.810 | 0.860 | 1.44 | 5.4 × 10⁻⁶ | 1.27 | 1.0 × 10⁻⁵ |
| ″ | rs2290225 | 127511564 | C | 0.506 | 0.523 | 1.07 | 0.26 | 0.505 | 0.571 | 1.31 | 5.5 × 10⁻⁶ | 1.19 | 4.8 × 10⁻⁵ |
| 9 | rs10116277 | 22071397 | T | 0.418 | 0.470 | 1.23 | 0.00066 | 0.454 | 0.516 | 1.28 | 2.2 × 10⁻⁵ | 1.26 | 6.0 × 10⁻⁸ |
| ″ | rs1333040 | 22073404 | T | 0.491 | 0.543 | 1.23 | 0.00067 | 0.550 | 0.608 | 1.27 | 6.3 × 10⁻⁵ | 1.25 | 1.6 × 10⁻⁷ |
| ″ | rs2383207 | 22105959 | G | 0.457 | 0.524 | 1.31 | 1.3 × 10⁻⁵ | 0.487 | 0.540 | 1.24 | 0.0003 | 1.27 | 1.9 × 10⁻⁸ |
| ″ | rs7025486 | 119798448 | A | 0.298 | 0.347 | 1.25 | 0.00063 | 0.286 | 0.320 | 1.17 | 0.012 | 1.21 | 2.9 × 10⁻⁵ |
| 12 | rs1671518 | 16280892 | G | 0.726 | 0.769 | 1.26 | 0.0014 | 0.719 | 0.757 | 1.21 | 0.0041 | 1.23 | 1.9 × 10⁻⁵ |
| ″ | rs10860944 | 102039897 | C | 0.190 | 0.220 | 1.20 | 0.015 | 0.198 | 0.240 | 1.28 | 0.00049 | 1.24 | 2.5 × 10⁻⁵ |
| 14 | rs4900514 | 99961084 | T | 0.645 | 0.667 | 1.10 | 0.14 | 0.608 | 0.689 | 1.43 | 1.3 × 10⁻⁸ | 1.26 | 2.7 × 10⁻⁷ |
| 15 | rs1471151 | 93274625 | T | 0.600 | 0.644 | 1.21 | 0.0032 | 0.580 | 0.625 | 1.21 | 0.0016 | 1.21 | 1.5 × 10⁻⁵ |
| ″ | rs8025525 | 93303007 | A | 0.541 | 0.594 | 1.24 | 0.00065 | 0.526 | 0.571 | 1.20 | 0.0020 | 1.22 | 4.4 × 10⁻⁶ |
| 17 | rs205043 | 11524523 | G | 0.297 | 0.345 | 1.24 | 0.00096 | 0.260 | 0.302 | 1.23 | 0.0016 | 1.24 | 5.1 × 10⁻⁶ |
| 19 | rs17207173 | 58769682 | T | 0.310 | 0.368 | 1.30 | 6.0 × 10⁻⁵ | 0.355 | 0.380 | 1.11 | 0.075 | 1.20 | 5.2 × 10⁻⁵ |
| 21 | rs2836470 | 38818203 | C | 0.851 | 0.880 | 1.28 | 0.0064 | 0.844 | 0.876 | 1.30 | 0.0021 | 1.29 | 3.9 × 10⁻⁵ |
| X | rs7052934 | 141294834 | C | 0.065 | 0.086 | 1.35 | 0.027 | 0.035 | 0.077 | 2.27 | 9.0 × 10⁻⁷ | 1.66 | 1.5 × 10⁻⁶ |

TABLE 3
Association results for the 19 GWAS lead SNPs in the AAA discovery
and follow up set 1.
Association results for the 19 follow-up SNPs in the discovery (1,292 cases and
30,503 controls) and follow-up sample set 1 (1,665 cases and 1,931 controls); both
separately and combined. The table shows the chromosome and position in NCBI
Build 36, the allele tested for association (EA), the OR and the P value.

| | | | | Discovery | | Follow-up | | | |
|---|---|---|---|---|---|---|---|---|---|
| | | | | set | | set 1 | | Combined | |
| Chr | SNP | Pos | EA | OR | P | OR | P | OR (95% CI) | P |
| 1 | rs487174 | 45782123 | T | 1.32 | 3.3 × 10⁻⁵ | 1.04 | 0.54 | 1.17 (1.07-1.28) | 0.00088 |
| ″ | rs1998064 | 228353751 | A | 1.25 | 8.9 × 10⁻⁶ | 0.97 | 0.63 | 1.13 (1.05-1.22) | 0.0018 |
| 5 | rs1372319 | 77945695 | T | 1.21 | 5.3 × 10⁻⁵ | 1.06 | 0.33 | 1.15 (1.07-1.23) | 0.00015 |
| ″ | rs959461 | 169812358 | G | 1.26 | 6.2 × 10⁻⁶ | 0.98 | 0.76 | 1.14 (1.06-1.23) | 0.00084 |
| 6 | rs9268832 | 32535767 | T | 1.19 | 5.3 × 10⁻⁵ | 0.99 | 0.83 | 1.10 (1.03-1.17) | 0.0032 |
| ″ | rs783166 | 161097227 | T | 1.35 | 7.4 × 10⁻⁶ | 0.96 | 0.56 | 1.16 (1.05-1.28) | 0.0029 |
| ″ | rs1652500 | 161112179 | C | 1.26 | 1.3 × 10⁻⁵ | 1.01 | 0.93 | 1.15 (1.06-1.25) | 0.00064 |
| 7 | rs7798936 | 95251189 | A | 1.20 | 1.7 × 10⁻⁵ | 0.95 | 0.4 | 1.10 (1.03-1.18) | 0.0039 |
| ″ | rs6979784 | 127703444 | G | 1.27 | 1.0 × 10⁻⁵ | 1.00 | 1 | 1.14 (1.06-1.24) | 0.00087 |
| ″ | rs2290225 | 127744838 | C | 1.19 | 4.8 × 10⁻⁵ | 0.99 | 0.54 | 1.01 (0.98-1.04) | 0.42 |
| 9 | rs7025486 | 123462224 | A | 1.21 | 2.9 × 10⁻⁵ | 1.28 | 1.2 × 10⁻⁵ | 1.24 (1.15-1.33) | 1.8 × 10⁻⁹ |
| 12 | rs1671518 | 16280892 | G | 1.23 | 1.9 × 10⁻⁵ | 1.07 | 0.19 | 1.16 (1.08-1.25) | 4.8 × 10⁻⁵ |
| ″ | rs10860944 | 102061560 | C | 1.24 | 2.5 × 10⁻⁵ | 0.97 | 0.62 | 1.11 (1.03-1.20) | 0.0054 |
| 14 | rs4900514 | 101040796 | T | 1.26 | 2.7 × 10⁻⁷ | 1.01 | 0.84 | 1.15 (1.07-1.23) | 5.5 × 10⁻⁵ |
| 15 | rs1471151 | 93345861 | T | 1.21 | 1.5 × 10⁻⁵ | 0.99 | 0.86 | 1.10 (1.03-1.17) | 0.0027 |
| ″ | rs8025525 | 93374243 | A | 1.22 | 4.4 × 10⁻⁶ | 1.00 | 0.99 | 1.11 (1.05-1.19) | 0.00068 |
| 17 | rs205043 | 11264682 | G | 1.24 | 5.1 × 10⁻⁶ | 1.04 | 0.5 | 1.16 (1.08-1.24) | 6.2 × 10⁻⁵ |
| 19 | rs17207173 | 58769682 | T | 1.20 | 5.2 × 10⁻⁵ | 1.02 | 0.72 | 1.12 (1.05-1.20) | 0.00082 |
| 21 | rs2836470 | 38819677 | C | 1.29 | 3.9 × 10⁻⁵ | 1.05 | 0.45 | 1.16 (1.07-1.27) | 6.0 × 10⁻⁴ |

TABLE 4
Association of rs7025486[A] with abdominal aortic aneurysm.

| Sample Set (n_{c}/nₐ)^{a} | f_{c}^{b} | fₐ^{b} | OR (95% CI) | P | Pₕₑₜ^{c} |
|---|---|---|---|---|---|
| Discovery samples | | | | | |
| Iceland^{d} (27,712/452) | 0.298 | 0.347 | 1.25 (1.10-1.42) | 0.00063 | |
| The Netherlands (Utrecht) (2,791/840) | 0.286 | 0.320 | 1.17 (1.04-1.33) | 0.012 | |
| Combined (30,503/1,292) | | | 1.21 (1.11-1.32) | 2.9 × 10⁻⁵ | |
| Follow up set 1 | | | | | |
| Belgium (266/172) | 0.227 | 0.253 | 1.15 (0.84-1.58) | 0.39 | |
| Canada (150/196) | 0.280 | 0.306 | 1.13 (0.82-1.58) | 0.45 | |
| New Zealand^{e} (848/1,144) | 0.226 | 0.273 | 1.28 (1.11-1.49) | 0.00097 | |
| UK (667/455) | 0.216 | 0.278 | 1.40 (1.15-1.70) | 0.0008 | |
| Combined (1,931/1,967) | | | 1.28 (1.15-1.41) | 3.6 × 10⁻⁶ | |
| Follow up set 2 | | | | | |
| Denmark (4,380/297) | 0.274 | 0.306 | 1.17 (0.98-1.41) | 0.087 | |
| The Netherlands (Nijmegen) (301/147) | 0.287 | 0.248 | 0.82 (0.60-1.12) | 0.22 | |
| US (Danville) (380/758) | 0.253 | 0.278 | 1.14 (0.93-1.39) | 0.2 | |
| US (Pittsburgh) (459/98) | 0.245 | 0.281 | 1.20 (0.85-1.70) | 0.3 | |
| Combined (5,520/1,300) | | | 1.11 (0.98-1.25) | 0.081 | |
| Replication (7,451/3,268) | | | 1.20 (1.11-1.30) | 3.8 × 10⁻⁶ | |
| Combined (37,954/4,559) | | | 1.21 (1.14-1.28) | 4.6 × 10⁻¹⁰ | 0.37 |

Association of rs7025486[A] with AAA in the two discovery sample sets and in eight follow-up sample sets of European descent.
^{a}The number of controls n_{c} and cases nₐ.
^{b}Frequency in controls f_{c} and in cases fₐ.
^{c}P value for the test of heterogeneity in the effect estimates.
^{d}536 ungenotyped Icelandic AAA cases were included in the analysis yielding n_{a,eff} = 632 and the P value was adjusted for relatedness of the Icelandic individuals by dividing the χ²-statistic by the genomic-control factor λ_{g} = 1.143.
^{e}This analysis includes additional 302 AAA cases only genotyped for rs7025486.

TABLE 5
Association of rs7025486-A with DAB2IP expression.

| Tissue | Probe | N | Effect | P |
|---|---|---|---|---|
| Blood | NM_032552 | 672 | 0.005 | 0.24 |
| Adipose | NM_032552 | 609 | −0.013 | 0.018 |
| Ascending aorta intima/media | 3187834 | 117 | 0.067 | 0.026 |
| Mammary artery intima/media | 3187834 | 88 | −0.057 | 0.016 |

Shown are the number of individuals with genotype and expression information (N), the observed effect on expression, and a P value. The P values for blood and adipose tissue have been adjusted for related of the individuals by dividing the corresponding χ²-statistic by 1.063 and 1.078, respectively.

TABLE 6
Association of rs7025486[A] with myocardial infarction.

| | | | | | |
|---|---|---|---|---|---|
| Sample Set (n_{c}/nₐ)^{a} | f_{c}^{b} | fₐ^{b} | OR (95% CI) | P | Pₕₑₜ^{c} |

Myocardial Infarction

| | | | | | |
|---|---|---|---|---|---|
| Iceland | 0.292 | 0.301 | 1.04 (0.97-1.12) | 0.27 | |
| (5,863/2,631)^{d} | | | | | |
| Italy | 0.208 | 0.240 | 1.21 (0.97-1.50) | 0.088 | |
| (383/637) | | | | | |
| New Zealand | 0.226 | 0.249 | 1.13 (0.94-1.36) | 0.18 | |
| (848/529) | | | | | |
| US (Atlanta) | 0.258 | 0.286 | 1.15 (0.95-1.39) | 0.14 | |
| (933/386) | | | | | |
| US (Baltimore) | 0.267 | 0.290 | 1.12 (0.88-1.43) | 0.35 | |
| (1,564/183) | | | | | |
| US (Durham) | 0.239 | 0.268 | 1.16 (1.00-1.36) | 0.049 | |
| (705/1191) | | | | | |
| US (Philadelphia) | 0.245 | 0.269 | 1.13 (0.92-1.38) | 0.23 | |
| (461/540) | | | | | |
| Combined | | | 1.09 (1.04-1.15) | 0.0012 | 0.73 |
| (10,757/6,096) | | | | | |

Early-onset myocardial infarction

| | | | | | |
|---|---|---|---|---|---|
| Iceland (5,863/723)^{e} | 0.292 | 0.326 | 1.17 (1.04-1.31) | 0.0071 | |
| Italy (383/194) | 0.208 | 0.245 | 1.24 (0.92-1.66) | 0.15 | |
| New Zealand | 0.226 | 0.178 | 0.74 (0.48-1.13) | 0.17 | |
| (848/73) | | | | | |
| US (Atlanta) | 0.258 | 0.318 | 1.34 (1.07-1.68) | 0.011 | |
| (933/223) | | | | | |
| US (Baltimore) | 0.267 | 0.288 | 1.11 (0.84-1.47) | 0.46 | |
| (1,564/132) | | | | | |
| US (Durham) | 0.239 | 0.273 | 1.20 (1.00-1.43) | 0.047 | |
| (705/595) | | | | | |
| US (Philadelphia) | 0.245 | 0.274 | 1.16 (0.89-1.52) | 0.27 | |
| (461/199) | | | | | |
| Combined | | | 1.18 (1.09-1.27) | 3.1 × 10⁻⁵ | 0.44 |
| (10,757/2,139) | | | | | |

Association of rs7025486[A] with MI and early-onset MI in seven sample sets of European descent.
^{a}The number of controls n_{c} and cases nₐ.
^{b}Frequency in controls f_{c} and in cases fₐ.
^{c}P value for the test of heterogeneity in the effect estimates.
^{d,e}4387 and 667 ungenotyped Icelandic MI and early-onset MI cases were included in the analysis, yielding n_{a,eff} = 4,077 and 925 respectively, and the corresponding P values were adjusted for relatedness of the Icelandic individuals by dividing the χ²-statistic by the genomic-control factor λ_{g} = 1.426 and 1.196, respectively.

TABLE 7
Association of rs7025486[A] with peripheral arterial disease,
venous thromboembolism and pulmonary embolism.

| | | | | | |
|---|---|---|---|---|---|
| Sample Set (n_{c}/nₐ)^{a} | f_{c}^{b} | fₐ^{b} | OR (95% CI) | P | Pₕₑₜ^{c} |

Peripheral Arterial Disease

| | | | | | |
|---|---|---|---|---|---|
| Iceland | 0.292 | 0.327 | 1.18 (1.08-1.28) | 0.00022 | |
| (5,863/1,575)^{d} | | | | | |
| Austria (423/458) | 0.249 | 0.266 | 1.09 (0.88-1.35) | 0.42 | |
| Denmark | 0.274 | 0.297 | 1.12 (0.96-1.30) | 0.14 | |
| (4,380/455) | | | | | |
| Italy (234/168) | 0.216 | 0.193 | 0.87 (0.62-1.23) | 0.44 | |
| New Zealand | 0.226 | 0.250 | 1.14 (0.92-1.41) | 0.22 | |
| (848/434) | | | | | |
| Sweden (143/204) | 0.252 | 0.277 | 1.14 (0.81-1.60) | 0.46 | |
| US (Danville) | 0.253 | 0.274 | 1.12 (0.89-1.40) | 0.34 | |
| (380/396) | | | | | |
| Combined | | | 1.14 (1.07-1.21) | 3.9 × 10⁻⁵ | 0.79 |
| (12,271/3,690) | | | | | |

Venous Thromboembolism

| | | | | | |
|---|---|---|---|---|---|
| Iceland | 0.292 | 0.319 | 1.14 (1.03-1.25) | 0.011 | |
| (5,863/1,019)^{e} | | | | | |
| Canada | | | | | |
| ACE (226/187) | 0.257 | 0.246 | 0.95 (0.68-1.30) | 0.73 | |
| PEDS (78/27) | 0.263 | 0.278 | 1.08 (0.54-2.16) | 0.83 | |
| Spain (888/675) | 0.177 | 0.196 | 1.13 (0.94-1.36) | 0.18 | |
| Combined | | | 1.12 (1.03-1.22) | 0.0079 | 0.71 |
| (7,055/1,908) | | | | | |

Pulmonary Embolism

| | | | | | |
|---|---|---|---|---|---|
| Iceland (5,863/479)^{f} | 0.292 | 0.333 | 1.21 (1.07-1.37) | 0.0026 | |
| Canada | | | | | |
| ACE (226/72) | 0.257 | 0.271 | 1.08 (0.70-1.65) | 0.74 | |
| PEDS (78/26) | 0.263 | 0.288 | 1.14 (0.56-2.29) | 0.72 | |
| Spain (888/234) | 0.176 | 0.218 | 1.30 (1.01-1.67) | 0.045 | |
| Combined | | | 1.20 (1.09-1.32) | 0.00030 | 0.97 |
| (7,055/811) | | | | | |

Association of rs7025486[A] with PAD, VTE and PE in several sample sets of European descent.
^{a}The number of controls n_{c} and cases nₐ.
^{b}Frequency in controls f_{c} and in cases fₐ.
^{c}P value for the test of heterogeneity in the effect estimates.
^{d,e,f}1,035, 1,626 and 901 ungenotyped Icelandic PAD, VT and PE cases were included in the analysis, with corresponding n_{a,eff} = 1,933, 1,554 and 775, and the P values were adjusted for relatedness of the Icelandic individuals by dividing the χ²-statistic by the genomic-control factor λ_{g} = 1.225, 1.319 and 1.207, respectively.

TABLE 8
Association of rs7025486[A] with venous thromboembolism
and with pulmonary embolism excluding individuals with
known CVD's.

| | | | | | |
|---|---|---|---|---|---|
| Sample Set (n_{c}/nₐ) | f_{c} | fₐ | OR (95% CI) | P | Pₕₑₜ |

Venous Thromboembolism

| | | | | | |
|---|---|---|---|---|---|
| Iceland (5,863/655) | 0.292 | 0.319 | 1.14 (1.02-1.28) | 0.027 | |
| Canada | | | | | |
| ACE (226/169) | 0.257 | 0.254 | 0.99 (0.71-1.37) | 0.94 | |
| Spain (888/654) | 0.177 | 0.200 | 1.15 (0.96-1.35) | 0.13 | |
| Combined | | | 1.13 (1.03-1.24) | 0.011 | 0.70 |
| (6,977/1,478) | | | | | |

Pulmonary Embolism

| | | | | | |
|---|---|---|---|---|---|
| Iceland (5,863/296) | 0.292 | 0.327 | 1.18 (1.01-1.37) | 0.034 | |
| Canada | | | | | |
| ACE (226/69) | 0.257 | 0.289 | 1.28 (0.77-1.81) | 0.44 | |
| Spain (888/228) | 0.176 | 0.221 | 1.31 (1.01-1.69) | 0.041 | |
| Combined (6,977/593) | | | 1.21 (1.07-1.37) | 0.0030 | 0.79 |

Association of rs7025486[A] with venous thromboembolism and with pulmonary embolism after excluding from the cases individuals with known incident of CAD, PAD (for all sample sets) or AAA, (for the Icelandic samples). Shown are the number of controls n_{c} and cases nₐ, the frequency in controls f_{c} and in cases fₐ, the OR with 95% CI, P value for test of association and the P value for the test of heterogeneity in the effect estimates. 1,140 (n_{a,eff} = 1,011) and 612 (n_{a,eff} = 482) un-genotyped Icelandic individuals with VTE and with PE were included in the analysis, respectively. The P value was adjusted for relatedness of the Icelandic individuals by dividing the χ² statistic by the genomic-control factor λ_{g} = 1.275 for the VTE and λ_{g} = 1.176 for the PE analysis.

### Example 2

### Study Populations

All studies were approved by relevant institutional review boards or ethics committees, and all participants provided written informed consent.

### AAA Discovery Samples

Iceland:

Icelandic individuals with AAA (defined as diameter of infarenal aorta of ≧30 mm) were recruited from a registry of individuals who were admitted at Landspitali University Hospital, in Reykjavik, Iceland, 1980-2006. AAA patients were either followed up or treated by intervention for emergency repair of symptomatic or ruptured AAA or for an elective repair by surgery or endovascular intervention. Subjects with AAA were enrolled as part of the CVD genetics program at deCODE. The 27,712 Icelandic controls used in the AAA GWAS were selected from among individuals who have participated in various GWA studies and who were recruited as part of genetic programs at deCODE. Individuals with known cardiovascular disease were excluded as controls.

Utrecht, The Netherlands:

The AAA sample set from Utrecht was recruited in 2007-2009 from eight centers in The Netherland, mainly when individuals visited their vascular surgeon in the polyclinic or, in rare cases, during hospital admission for elective or emergency AAA surgery. An AAA was defined as an infrarenal aorta≧30 mm. The sample set comprised 89.9% males, with a mean AAA diameter of 58.4 mm, 61.7% had received surgery, of which 8.1% was after rupture. The Dutch controls used in the AAA GWAS were recruited as part of the Nijmegen Biomedical Study and the Nijmegen Bladder Cancer Study (see http://dceg.cancer.gov/icbc/membership.html). The details of these studies were reported previously^{1,2}.

### AAA Follow Up Samples

New Zealand:

Individuals from New Zealand with AAA were recruited from the Otago-Southland region of the country, the vast majority (>97%) being of Anglo-European ancestry as reported previously³. Approximately 80% of these individuals had undergone surgical AAA repair (typically AAA's>50 mm in diameter). The control group consisted of elderly individuals with no previous history of vascular disease from the same geographical region. An abdominal ultrasound scan excluded concurrent abdominal aortic aneurysm from the control group and Anglo-European ancestry was required for inclusion. Controls were also asymptomatic for PAD and had ankle brachial indexes >1.

United Kingdom:

UK individuals with AAA referred to vascular surgeons at 93 UK hospitals were entered into the UK Small Aneurysm Trial. For the purpose of the current study, those individuals randomized to surveillance in the UK Small Aneurysm Trial with AAA diameter 40-55 mm were selected as a case group, although some cases had been monitored before their aneurysm reached the 40-mm threshold for the trial. Mean AAA diameter at baseline was 45 mm (32-55 mm)⁴. Controls were of European descent, recruited from England⁵.

Belgium and Canada:

These sample-sets include individuals with AAA who were admitted either for emergency repair of ruptured AAA or for an elective surgery to the University Hospital of Liege (Liege, Belgium) and to Dalhousie University Hospital (Halifax, Canada). AAA was defined as an infrarenal aortic diameter of 30 mm or greater. Details of these case-control sets have been previously reported⁶. All individuals were of European descent. Approximately 40% of individuals with AAA had a family history of AAA. Control samples (51% males) of European descent were obtained from spouses of individuals with AAA or from individuals admitted to the same hospitals for reasons other than AAA.

Nijmegen, The Netherlands:

Individuals with AAA were recruited through a primary screening program of men aged 60-80 years. Three neighbouring districts in the east of Netherlands were selected for the AAA screening study. A diagnosis of AAA was established when an aortic diameter of 30 mm was measured. Individuals with an aortic diameter of 50 mm (app. 20% of all cases) were referred to a regional hospital for elective aneurysm repair. The control group was derived from the same screening program. For each case at least one age-match control was selected.

Pittsburgh, Pa., US:

Patients admitted to the University Hospital of Pittsburgh for either elective or emergency surgery for AAA were selected for the study⁷. The cases consisted of individuals of European origin entering Presbyterian University Hospital (PUH) in Pittsburgh for aneurysm resection from 1986 to 1991. Mean aneurysm diameter was 58.6±15.7 mm. Controls were selected from participants of the cardiac catheterization study program at the University of Pennsylvania Medical Center in Philadelphia (PENN CATH). The control group represents individuals who did not have significant luminal stenosis on coronary angiography (luminal stenosis >50%) or a history of myocardial infarction.

Danville, Pa., US:

AAA patients were enrolled through the Geisinger Clinic Department of Vascular Surgery at Geisinger Medical Center, Danville, Pa.⁸. AAA cases were defined as infrarenal aortic diameter≧30 mm as revealed by abdominal imaging. An unselected control group was obtained through the Geisinger MyCode Project, a cohort of Geisinger Clinic primary care patients recruited for genomic studies. The MyCode controls were matched for age distribution and gender to the Geisinger Vascular Clinic AAA cases.

Denmark:

The Danish AAA-samples were recruited from two population-based screening programmes for 65-74 year old men in 1994-1998⁹ and 2008-2009 (Clinical trials: NCT00662480). In both cohorts, an AAA was defined as an infrarenal aorta≧30 mm—in average 40.5 mm, and 12% were above 50 mm at diagnosis. None were ruptured, and the mean age was 68.2 years. The Danish controls come from the randomised population-based intervention study (Inter99) which has been described in details elsewhere¹⁰.

### Peripheral Arterial Disease Samples

Iceland:

Patients were recruited through a registry of individuals diagnosed with PAD during the year 1998-2006, at the Landspitali University Hospital (Reykjavik, Iceland). The PAD diagnosis was confirmed by vascular imaging or segmental pressure measurements. Subjects with PAD were enrolled over a nine year period as part of the CVD genetics program at deCODE. A new set of 5,863 Icelandic population controls without history of vascular diseases and not overlapping with the set of 27,712 controls used in the GWA study, were genotyped for the risk variant and used in the association tests of PAD. These individuals were selected among individuals that had participated in various genetic programs at deCODE genetics and that have not been genotyped with any of the SNP chips used.

New Zealand:

Patients were recruited from the Otago-Southland region of the country, and PAD was confirmed by an ankle brachial index<0.7, pulse volume recordings and angiography/ultrasound imaging. An abdominal ultrasound scan excluded concurrent AAA from the PAD group. Controls were the same vascular disease-free individuals as described above for the New Zealand AAA sample set.

Austria:

Patients and controls were recruited through the Linz Peripheral Arterial Disease (LIPAD) study during 2000 to 2002, at the St. John of God Hospital, Department of Surgery (Linz, Austria). Of the recruited patient, all with chronic atherosclerotic occlusive disease of the lower extremities associated with typical symptoms, such as claudication or leg pain on exertion, rest pain, or minor or major tissue loss, were included in this study on the basis of the final clinical diagnosis established by the attending vascular surgeons. The diagnosis was verified by interview, physical examination, non-invasive techniques, and angiography¹¹. Control subjects were patients at the same hospital that fulfilled the following criteria: no clinical indication of PAD by history and physical examination; systolic brachial blood pressure equal to or less than the blood pressure in each of the right and left anterior tibial and posterior tibial arteries (ie, ABI≧1.0)¹¹.

Italy:

Patients and controls were recruited among subjects consecutively admitted to the Department of Medicine of the A. Gemelli University Hospital of Rome, from 2000 to 2001. Diagnosis of PAD was performed in accordance with established criteria¹². All patients had an ankle/arm pressure index lower than 0.8 and were at Fontaine's stage II, with intermittent claudication and no rest pain or trophic lesions¹³.

Denmark:

Patients with PAD were consecutively included during November 1999 to January 2004. The diagnosis was established from typical findings in clinical investigation (intermittent claudication, rest pain, ulcer or gangrene, and ankle-brachial-index<0.9). All patients were enrolled at Vascular Surgery Department, Viborg Hospital, Denmark¹⁴. The Danish controls come from the Inter99 previously described¹⁰.

Sweden:

Patients and controls were recruited at the Department of Vascular Diseases at Malmo University Hospital (Malmö, Sweden). The diagnosis of critical limb ischemia was made in accordance with TransAtlantic Inter-Society Consensus scientific criteria¹⁵ of ulceration, gangrene, or rest pain caused by PAD proven by ankle pressure (<50 to 70 mm Hg), reduced toe pressure (<30 to 50 mm Hg), or reduced transcutaneous oxygen tension. Diagnosis was confirmed by an experienced vascular surgery consultant and toe pressure measurements in patients with arteries in the affected leg that were non-compressible and the ankle pressure was >50 to 70 mm Hg. The control group consisted of healthy individuals included in a health screening programme for a preventive medicine project. None of those had symptomatic PAD¹⁶.

Danville, Pa., US:

Individuals with PAD were enrolled through the Geisinger Clinic Department of Vascular Surgery⁸. PAD subjects had an ankle brachial index (ABI)≦0.85 in at least one leg and were confirmed to be AAA-free (infrarenal aortic diameter≦2 cm) based on abdominal imaging carried out within the preceding 5 years. Controls were the same individuals as previously described above for the Danville AAA group

### Myocardial Infarction Samples

Iceland:

The GWAS of MI in Icelanders by deCODE has been previously described¹⁷. Briefly, cases were initially identified from a registry of over 10,000 individuals who suffered an MI before the age of 75 in Iceland between 1981 and 2002 and satisfied the MONICA criteria¹⁸, or had an MI discharge diagnosis from the Landspitali University Hospital (LUH) (Reykjavik, Iceland) between 2003 and 2005. These patients were recruited through the cardiovascular disease genetics program at deCODE. In 2009 the sample set was expanded to include additional subjects that were given the discharge diagnosis of MI at LUH between 1987 and 2007 and had donated blood through various genetic programs at deCODE genetics. Controls were the same individuals as described above for the Icelandic PAD sample set.

Atlanta, Ga., US:

The study participants were enrolled at the Emory University Hospital, Crawford Long Hospital, the Emory Clinic and Grady Memorial Hospital, all in Atlanta, Ga., through the Emory Genebank Study. The Emory Genebank Study was designed to investigate the association of biochemical and genetic factors to CAD in subjects undergoing cardiac catheterization. For the purpose of the current study, subjects with current or prior history of MI were defined as cases. Subjects with no or minimal CAD on cardiac catheterization and no prior history of MI or CAD were defined as controls. Information on ethnicity was self-reported.

Durham, N.C., US:

The study participants were enrolled at Duke University Medical Center (Durham, N.C.) through the CATHGEN biorepository, consisting of subjects above 18 years of age, recruited sequentially through the cardiac catheterization laboratories from 2001-2005. For purposes of this study, cases of MI were defined as those having a history of MI (by self-report and corroborated by review of medical records), or having suffered an MI during the study follow-up period. Subjects with no prior history of MI or CAD and no or minimal CAD on cardiac catheterization were defined as controls.

Baltimore, Md., US:

Subjects were recruited from the ongoing prospective family study (the Johns Hopkins GeneSTAR Study) which was designed to determine the environmental and genetic causes of chronic and cardiovascular diseases. Probands with documented MI and angiographically demonstrated coronary artery stenosis≧50% of the vessel internal diameter in at least 1 vessel before the age of 60 were identified at the time of hospitalization in any of 10 Baltimore area hospitals between 1984 and 2006 and their healthy siblings without CAD or any vascular diseases ages 30-59 were enrolled and followed for incident MI. For purposes of this study, cases were defined as those new onset-MI during 5-25 years of follow-up. Standard criteria for MI (elevations of cardiac enzymes and electrocardiographic changes) were adjudicated from medical record reviews of all siblings. Subjects who remained healthy with no CAD during follow-up were defined as controls. Ethnicity was self-reported.

Philadelphia, Pa., US:

The study participants from Philadelphia were enrolled at the University of Pennsylvania Medical Center through the PENN CATH study which recruited a consecutive sample of patients undergoing cardiac catheterization at the University of Pennsylvania Medical Center (Philadelphia, Pa.) between July 1998 and March 2003. For the purpose of the current study we selected from the PENN CATH study individuals diagnosed with MI based on criteria for acute MI in terms of elevations of cardiac enzymes and electrocardiographic changes, or a self-reported history of MI. Ethnicity information was self-reported. Controls were the same individuals as described above for the Pittsburgh AAA sample set.

New Zealand:

Individuals who suffered an MI were identified from a registry of CAD patients with angiographically proven coronary artery stenosis≧50% of the vessel internal diameter in at least 1 vessel. The age matched controls had no history of ischemic heart disease, including angina pectoris. All subjects were recruited from the Otago-Southland region of the country and ethnicity information was self-reported. Controls were the same individuals as described above for the New Zealand AAA sample set.

Italy.

The subjects from Verona were enrolled into the Verona Heart Study (Verona, Italy) an ongoing study aimed at identifying new risk factors for CAD and MI in a population of subjects with angiographic documentation of their coronary vessels¹⁹. Information on MI diagnoses was gathered through medical records showing diagnostic electrocardiogram and enzyme changes, and/or the typical sequelae of MI on ventricular angiography. Control subjects had normal coronary arteries, being submitted to coronary angiography for reasons other than CAD. Controls with history or clinical evidence of atherosclerosis in vascular districts beyond the coronary bed were excluded.

### Venous Thromboembolism Samples

Iceland:

All patients with VTE diagnosed objectively by imaging techniques at the three

Reykjavik acute care hospitals during 1987 to 2002 were included. These hospitals serve as acute care hospitals for about ⅔ of the Icelandic population and referral hospitals for the whole nation. The accepted imaging techniques were: compression ultrasound, venogram, ventilation perfusion (VQ-) scan, pulmonary angiograms, computerized pulmonary angiograms or autopsy confirmed VTE. The cases were identified by a computer search of the following ICD diagnostic codes: ICD-9: 415, 415.1, 451, 451.1, 451.2, 451.8, 451.9, 453.1, 453.8, 459.1. ICD-10: 126, 126.9, 180, 180.1, 180.2, 180.3, 180.9, 182.1, 182.8, 187.0. The hospital charts of all identified patients were then reviewed by experienced physicians in order to exclude erroneous diagnoses (e.g. superfical thrombophlebitis, arterial emboli). Cases were also eliminated if a confirmatory imaging report could not be found. Information on the presence of other cardiovascular diseases (i.e. CAD, AAA and PAD) at the time of diagnosis is not available. However, the Icelandic CAD and AAA lists include the majority of all cases diagnosed in Iceland over a specific time period (CAD over 27,000 cases diagnoses 1981-2009, and AAA over 1,100 cases diagnosed 1980-2006). Comparison of these lists with the VTE case list shows that 2.1% have also been diagnosed with AAA and 27.4% with CAD. We have less complete information on individuals diagnosed with PAD in Iceland but based on the information we have 5.8% of the VTE cases have diagnosed with PAD. These numbers probably represent an overestimate as many VTE cases presented with their symptoms of CAD, AAA and PAD long after their diagnosis of VTE. Controls were the same individuals as described above for the Icelandic PAD sample set.

Canada: ACE study: Cases were recruited from the Thrombosis Clinic at the Ottawa Hospital which serves as a referral basis for a community of approximately 700,000 people. Consecutive patients with at least one objectively confirmed idiopathic deep vein thrombosis (DVT) or pulmonary embolism, who had been treated for at least 3 months, were eligible for inclusion²⁰. Patients were excluded if they had a malignant disorder. History of CAD and PAD was documented for the majority of cases at the time of recruitment. Of the VTE cases 6.9% had CAD and 4.9% PAD. PEDS study: Consecutive patients presenting with symptoms or signs suspected by a physician of being caused by acute pulmonary embolism (acute onset of new or worsening shortness of breath, chest pain, hemoptysis, presyncope or syncope) were eligible for the study, Of those fulfilling the inclusion criterion the following were excluded: 1) deep vein thrombosis or pulmonary embolism diagnosed within the previous 3 months; 2) no change in severity of pulmonary symptoms within the previous two weeks; 3) use of therapeutic doses of parenteral anticoagulants for greater than 48 hours; 4) Co-morbid condition making life expectancy less than three months 5) contraindication to contrast media; 6) a need for long-term use of anticoagulants; 7) pregnancy; 8) age less than 18 years; 9) refusal to give informed consent; and 10) geographic inaccessibility to follow-up 11) unable to give informed consent; 12) Spiral CT or VQ scan in the previous 7 days; and 13) previous enrollment in PEDS trial. Information on other cardiovascular diseases at the time of case recruitment was not available. Controls: Friends of cases were recruited as control individuals and they were matched to cases by sex, ethnicity, and age. Controls were excluded if they had prior VTE or recent malignant disease.

Spain: The patients were enrolled from the files of the anticoagulation clinics in 4 hospitals in Spain: Hospital General Universitario (Murcia), Hospital de la Santa Creu i Sant Pau (Barcelona), Hospital Clinico Universitario (Salamanca), and Clinica Universitaria de Navarra (Pamplona). The study includes unrelated individuals of European descent with a first, objectively confirmed episode of venous thromboembolism before the age of 75 years. All cases were diagnosed appropriately by clinical probability, D-dimer levels, compression ultrasonography, ventilation perfusion lung scan, and, when necessary, phlebography or pulmonary angiography. Patients with known malignant disorders were excluded. Of the VTE cases 3.3% had a history of CAD and 0.8% history of PAD. The control group of our study includes unrelated individuals without a history of vascular or thromboembolic disease. These controls were randomly selected among 2 sources: blood donors and traumatology and ophthalmology patients matched by age, sex, race, and geographic distribution with the cases²¹.

TABLE 9
Characteristics of study populations.

| | Controls | | | Cases | | | |
|---|---|---|---|---|---|---|---|
| Sample Set | n_{c} | Women (%) | Age (SD) | nₐ | Women (%) | Age (SD) | Ref. |

Abdominal aortic aneurysm (AAA)

| | | | | | | | |
|---|---|---|---|---|---|---|---|
| Iceland | 27,712 | 62 | 50.8 (21.4) | 452 | 26 | 75.5 (8.3) | 1 |
| Netherlands (Utrecht) | 2,791 | 40 | 58.4 (10.2) | 840 | 10 | 68.1 (8.3) | 2, 3 |
| Belgium | 267 | 32 | na | 176 | 11 | na | 1, 4 |
| Canada | 155 | 80 | na | 206 | 26 | na | 1, 4 |
| New Zealand | 848 | 35 | 69.3 (6.6) | 1,144 | 20 | 73.9 (8.9) | 1, 5 |
| UK | 667 | na | na | 476 | 17 | 69.1 (4.4) | 6, 7 |
| Denmark | 4,380 | 54 | 45.2 (7.9) | 323 | 0 | 68.2 | 8, 9 |
| Netherlands (Nijmegen) | 324 | 0 | 69.0 | 149 | 0 | 68.8 | |
| US (Danville) | 442 | 22 | 72 (9) | 793 | 20 | 74 (8) | 10 |
| US (Pittsburg) | 499 | na | na | 100 | 25 | na | 11 |

Myocardial infarction (MI)

| | | | | | | | |
|---|---|---|---|---|---|---|---|
| Iceland | 27,712 | 62 | 50.8 (21.4) | 2,489 | 29 | 68.6 (10.5) | 12 |
| Italy | 390 | 33 | 59.1 (12.3) | 637 | 22 | 60.0 (11.7) | 13, 14 |
| New Zealand | 848 | 35 | 69.3 (6.6) | 529 | 29 | 64.1 (10.3) | 13, 15 |
| US (Atlanta) | 1,249 | na | na | 386 | 22 | 63.6 (10.0) | 12 |
| US (Baltimore) | 1,565 | 59 | 45.3 (12.2) | 183 | 15 | 55.3 (8.9) | 12 |
| US (Durham) | 736 | 50 | 57.1 (11.9) | 1,191 | 24 | 61.1 (11.0) | 12 |
| US (Philadelphia) | 498 | 46 | 58.3 (11.9) | 540 | 28 | 61.8 (10.8) | 12 |

Peripheral artery disease (PAD)

| | | | | | | | |
|---|---|---|---|---|---|---|---|
| Iceland | 27,712 | 62 | 50.8 (21.4) | 1,477 | 39 | 72.5 (10.9) | 1 |
| Austria | 433 | 29 | 67.3 (10.7 | 487 | 30 | 68.5 (11.0) | 16 |
| Denmark | 4,380 | 54 | 45.2 (7.9) | 464 | 43 | 65.9 (9.6) | 17 |
| Italy | 242 | 31 | 72.7 (6.3) | 181 | 31 | 72.9 (9.3) | 1, 18 |
| New Zealand | 848 | 35 | 69.3 (6.6) | 450 | 43 | 70.5 (9.7) | 1 |
| Sweden | 143 | na | na | 206 | na | na | 1, 19 |
| US (Danville) | 442 | 22 | 72 (9) | 438 | 40 | 69 (10) | |

Venous thromboembolism (VTE)

| | | | | | | | |
|---|---|---|---|---|---|---|---|
| Iceland | 27,712 | 62 | 50.8 (21.4) | 946 | 56 | 70.2 (15.3) | |
| Canada | 226 | 40 | 57.2 (15.5) | 214 | 53 | 56.5 (15.3) | 20 |
| Spain | 1,018 | 50 | 49 (36-62) | 1,018 | 50 | 47 (35-63) | 21 |

Intracranial Aneurysm (IA)

| | | | | | | | |
|---|---|---|---|---|---|---|---|
| Iceland | 27,712 | 62 | 50.8 (21.4) | 174 | 64 | 61.4 (14.4) | 1 |
| Finland | 312 | 49 | 58.9 (12.1) | 321 | 52 | 48.2 (12.4) | 22 |
| Netherlands | 915 | 38 | 48 (12.7) | 646 | 66 | 49.5 (12.9) | 1 |

Ischemic stroke (IS)

| | | | | | | | |
|---|---|---|---|---|---|---|---|
| Iceland | 27,712 | 62 | 50.8 (21.4) | 2,225 | 45 | 74.7 (12.1) | 23 |

1) Helgadottir et al. Nat Gen 40, 217-224 (2008),
2) Kiemeney, L. A. et al. Nat Gen 40, 1307-12 (2008),
3) Wetzels, J. F. et al. Kidney Int 72, 632-7 (2007),
4) Ogata et al. J Vasc Surg 41, 1036-1042 (2007),
5) Jones et al. Clin Chem 53, 679-685 (2007),
6) Brady et al. Circulation 110, 16-21 (2004),
7) Stefansson, H. et al. Nature 460, 744-7 (2009),
8) Lindholt JS et al. BMJ Apr 2; 330(7494): 750 (2005),
9) Jörgensen et al. Eur J Cardiovasc Prev Rehabil. Oct; 10(5): 377-86. (2003),
10) Elmore et al. J Vasc Surg. Jun; 49(6): 1525-31 (2009),
11) St Jean, P. L. et al. Ann Hum Genet 59, 17-24 (1995),
12) Helgadottir et al. Science 316, 1491-1493 (2007),
13) Gudbjartsson et al. Nat Gen 40, 609-615 (2008),
14) Girelli et al. N Engl J Med 343, 774-780 (2000),
15) Jones et al. Arterioscler Thromb Vasc Biol 28, 764-770 (2008),
16) Mueller et al. J Vasc Surg 41, 808-815 (2005),
17) Joensen et al. Atherosclerosis 196, 937-942 (2008),
18) Flex et al. Eur J Vasc Endovasc Surg 24, 264-268 (2002),
19) Barani et al. J Vasc Surg 42, 75-80 (2005),
20) Wells, P. S. et al. Thromb Haemost 90, 829-34 (2003),
21) Corral J et al. Blood. 2006; 108: 177-183,
22) Weinsheimer et al. Stroke 38, 2670-2676 (2007),
23) Gretarsdottir et al. Ann Neurol 64, 402-409 (2008).

### Genotyping

Illumina Genome-Wide Genotyping:

The Icelandic and Dutch case and control samples used in the GWA AAA study were assayed with the Illumina HumanHap300, HumanHapCNV370 or HumanHap610 bead chips (Illumina, SanDiego, Calif., USA). Only SNPs present on all chips were included in the analysis and SNPs were excluded if they had (a) yield lower than 95% in cases or controls, (b) minor allele frequency less than 1% in the controls, or (c) showed significant deviation from Hardy-Weinberg equilibrium in the controls (P<0.0001). These criteria were applied separately to genotype data from each of the chip type used and SNPs that showed significant deviation (P<0.0001 in an ANOVA test) in frequency between the chips were excluded from the analysis. Any samples with a call rate below 98% were excluded from the analysis. The final analysis included 293,677 SNPs present on all three chips. The UK control samples were genotyped as part of the S-Gene Plus cohort using the Illumina HumanHap300 and HumanHap550 BeadChips⁵. New Zealand AAA and control samples were genotyped using Affymetrix SNP 6.0 arrays and the imputation of ungenotyped SNPs was done using the IMPUTE²⁴ software and the HapMap (NCBI Build 36 (db126b)) CEU data as reference²⁵.

Single SNP Genotyping:

Most single SNP genotyping for all samples was carried out at deCODE genetics (Reykjavik, Iceland) applying the same platform to all populations studied. All single SNP genotyping was carried out using the Centaurus (Nanogen) platform²⁶. The quality of each Centaurus SNP assay was evaluated by genotyping each assay on the CEU samples and comparing the results with the HapMap data. All assays had mismatch rate <0.5%. Additionally, all markers were re-genotyped on more than 10% of samples typed with the Illumina platform resulting in an observed mismatch in less than 0.5% of samples. Single SNP genotyping of the additional 302 AAA cases from New Zealand only genotyped for rs7025486 and not included in stage 1 replication, was done using a TaqMan assay (Applied Bio-sciences probe #C_{—}29006014_{—}10) at the New Zealand site.

Expression of DAB2IP and rs7025486

Whole Blood and Adipose:

The correlation between rs7025486[A] and the mRNA expression of DAB2IP was tested in adipose tissue and in whole blood from 674 and 1,002 Icelandic individuals, respectively. The collection of the tissue samples and the measurement of the expression was described previously²⁷. Of those individuals, 609 with adipose tissue sample and 672 with blood sample had been genotyped for rs7025486 and were used for the analysis. For each variant and each expression trait at the corresponding loci, the correlation was tested by regressing the mean logarithm (log₁₀) expression ratio (MLR) on the number of copies of the risk variant A a person carries. The effect of age and sex was taken into account by including age, sex and age×sex terms as an explanatory variables in the regression analysis. When analyzing expression in blood, adjustment for differential cell count was also included. The P values were adjusted for relatedness of the individuals by simulating genotypes through the Icelandic genealogy as previously described²⁸. The corresponding adjustment factors for the χ²-statistic were 1.063 and 1.078 for adipose tissue and blood, respectively. The gene expression data is available from the GEO database under the accession numbers GSE7965 and GPL3991. The study from which the expression data was derived was approved by the National Bioethics Committee (NBC01-033) and the Icelandic Data Protection Authority (DPA).

Aorta and Mammary Artery:

The aorta and mammary artery tissue samples were from the ASAP study that includes patients undergoing heart-valve surgery at Karolinska University Hospital, Stockholm, Sweden. Biopsies were obtained at surgery from dilated and non-dilated ascending aorta (117 samples; 25.6% females, mean age=61.6+/−11.3SD) and from mammary artery (88 samples; 37.5% females, mean age=65.0+/−11.3SD). The intima/media layer was isolated by adventicectomy, incubated with RNAlater (Ambion) and homogenized with a FastPrep (Qbiogene, Irvine, Calif.) using Lysing Matrix D tubes (Invitro cat. no. 6913-100). Total RNA was isolated using Trizol (BRL-Life Technologies) and RNeasy Mini kit (Qiagen) as a cleanup including treatment with RNase-free DNase set (Qiagen) according the manufacturer's instructions. The quality of RNA was analyzed with an Agilent 2100 bioanalyzer (Agilent Technologies Inc., Paolo Alto, Calif., USA) and quantity was measured by a NanoDrop (Thermo Scientific). RNA from each labelled vessel sample, altogether 205 samples, was hybridized to Affymetrix ST 1.0 exon array. Array images were processed using the RMA algorithm as described previously²⁹ to obtain single-colour log₂ expression values. The hybridizations went through standard QC process according to Affymetrix standards. Meta probe sets were analyzed from the extended set of Affymetrix defined probe groups. The expression of DAB2IP (probe 3187834) was tested for correlation with rs7025486 by regressing the age and sex adjusted expression values on the number of copies of the risk allele A an individual carries. This study was approved by the Ethics Committee at the Karolinska Institut and patients were included after informed, written and signed consent.

### REFERENCES

1. Kiemeney, L. A. et al. Sequence variant on 8q24 confers susceptibility to urinary bladder cancer. Nat Genet. 40, 1307-12 (2008).
2. Wetzels, J. F., Kiemeney, L. A., Swinkels, D. W., Willems, H. L. & den Heijer, M. Age- and gender-specific reference values of estimated GFR in Caucasians: the Nijmegen Biomedical Study. Kidney Int 72, 632-7 (2007).
3. Helgadottir, A. et al. The same sequence variant on 9p21 associates with myocardial infarction, abdominal aortic aneurysm and intracranial aneurysm. Nat Genet. 40, 217-24 (2008).
4. Brady, A. R., Thompson, S. G., Fowkes, F. G., Greenhalgh, R. M. & Powell, J. T. Abdominal aortic aneurysm expansion: risk factors and time intervals for surveillance. Circulation 110, 16-21 (2004).
5. Stefansson, H. et al. Common variants conferring risk of schizophrenia. Nature 460, 744-7 (2009).
6. Ogata, T. et al. Genetic analysis of polymorphisms in biologically relevant candidate genes in patients with abdominal aortic aneurysms. J Vasc Surg 41, 1036-42 (2005).
7. St Jean, P. L. et al. Characterization of a dinucleotide repeat in the 92 kDa type IV collagenase gene (CLG4B), localization of CLG4B to chromosome 20 and the role of CLG4B in aortic aneurysmal disease. Ann Hum Genet. 59, 17-24 (1995).
8. Elmore, J. R. et al. Identification of a genetic variant associated with abdominal aortic aneurysms on chromosome 3p12.3 by genome wide association. J Vasc Surg 49, 1525-31 (2009).
9. Lindholt, J. S., Juul, S., Fasting, H. & Henneberg, E. W. Screening for abdominal aortic aneurysms: single centre randomised controlled trial. Bmj 330, 750 (2005).
10. Jorgensen, T. et al. A randomized non-pharmacological intervention study for prevention of ischaemic heart disease: baseline results Inter99. Eur J Cardiovasc Prey Rehabil 10, 377-86 (2003).
11. Mueller, T. et al. Factor V Leiden, prothrombin G20210A, and methylenetetrahydrofolate reductase C677T mutations are not associated with chronic limb ischemia: the Linz Peripheral Arterial Disease (LIPAD) study. J Vasc Surg 41, 808-15 (2005).
12. Suggested standards for reports dealing with lower extremity ischemia. Prepared by the Ad Hoc Committee on Reporting Standards, Society for Vascular Surgery/North American Chapter, International Society for Cardiovascular Surgery. J Vasc Surg 4, 80-94 (1986).
13. Flex, A. et al. The −174 G/C polymorphism of the interleukin-6 gene promoter is associated with peripheral artery occlusive disease. Eur J Vasc Endovasc Surg 24, 264-8 (2002).
14. Joensen, J. B. et al. Can long-term antibiotic treatment prevent progression of peripheral arterial occlusive disease? A large, randomized, double-blinded, placebo-controlled trial. Atherosclerosis 196, 937-42 (2008).
15. Dormandy, J. A. & Rutherford, R. B. Management of peripheral arterial disease (PAD). TASC Working Group. TransAtlantic Inter-Society Consensus (TASC). J Vasc Surg 31, S1-S296 (2000).
16. Barani, J., Nilsson, J. A., Mattiasson, I., Lindblad, B. & Gottsater, A. Inflammatory mediators are associated with 1-year mortality in critical limb ischemia. J Vasc Surg 42, 75-80 (2005).
17. Helgadottir, A. et al. A common variant on chromosome 9p21 affects the risk of myocardial infarction. Science 316, 1491-3 (2007).
18. Nomenclature and criteria for diagnosis of ischemic heart disease. Report of the Joint International Society and Federation of Cardiology/World Health Organization task force on standardization of clinical nomenclature. Circulation 59, 607-9 (1979).
19. Girelli, D. et al. Polymorphisms in the factor VII gene and the risk of myocardial infarction in patients with coronary artery disease.N Engl J Med 343, 774-80 (2000).
20. Wells, P. S. et al. The ACE D/D genotype is protective against the development of idiopathic deep vein thrombosis and pulmonary embolism. Thromb Haemost 90, 829-34 (2003).
21. Corral, J. et al. A nonsense polymorphism in the protein Z-dependent protease inhibitor increases the risk for venous thrombosis. Blood 108, 177-83 (2006).
22. Weinsheimer, S. et al. Association of kallikrein gene polymorphisms with intracranial aneurysms. Stroke 38, 2670-6 (2007).
23. Adams, H. P., Jr. et al. Classification of subtype of acute ischemic stroke. Definitions for use in a multicenter clinical trial. TOAST. Trial of Org 10172 in Acute Stroke Treatment. Stroke 24, 35-41 (1993).
24. Marchini, J., Howie, B., Myers, S., McVean, G. & Donnelly, P. A new multipoint method for genome-wide association studies by imputation of genotypes. Nat Genet. 39, 906-13 (2007).
25. Frazer, K. A. et al. A second generation human haplotype map of over 3.1 million SNPs. Nature 449, 851-61 (2007).
26. Kutyavin, I. V. et al. A novel endonuclease IV post-PCR genotyping system. Nucleic Acids Res 34, e128 (2006).
27. Emilsson, V. et al. Genetics of gene expression and its effect on disease. Nature 452, 423-8 (2008).
28. Stefansson, H. et al. A common inversion under selection in Europeans. Nat. Genet. 37, 129-37 (2005).
29. Irizarry, R. A. et al. Exploration, normalization, and summaries of high density oligonucleotide array probe level data. Biostatistics 4, 249-64 (2003).
30. Sabeti, P. C. et al. Genome-wide detection and characterization of positive selection in human populations. Nature 449, 913-8 (2007).

### Example 3

The association of genotyped markers located near rs7025486 was tested in Icelandic and Dutch samples. This was done for the vascular phenotypes AAA, MI, early onset MI, PAD, VTE and PE. Results are shown in Table 10. Many of the genotyped markers are not in very strong linkage disequilibrium with rs7025486; as a consequence, their apparent association with the vascular phenotypes is not as strong as that observed for rs7025486.

We also investigated the association of surrogate markers of rs7025486 with the vascular disorders AAA, MI, early-onset MI, PAD, VTE and PE) based on imputed genotypes. The imputed markers are all correlated with rs7025486 by r²-values greater than 0.2. Genotypes were imputed based on genotype data from the HapMap CEU v24 and 1000 Genomes projects. All P-values were corrected base on genomic controls. We estimated OR and frequency for the predicted risk allele, i.e. the allele that is positively correlated with the A risk allele of rs7025786.

Results are shown in Table 11. As can be seen, those surrogate markers that are in strong LD with rs7025786 give stronger association results than those surrogates that are not as tightly correlated with rs7025486. Obviously, observed P-values of association are not as strong for a study of a single population as would be observed using a larger data set. In other words, larger data sets may be needed to detect an association that gives a p-value of less than 0.05 for some of the markers. Nevertheless, the data illustrates that surrogates do capture the association originally observed for rs7025486, as shown by the observed risk values. In general, the more strongly correlated the surrogate marker is, the higher the observed risk is, which approaches that of rs7025486 as the correlation between the markers approaches unity.

TABLE 10
Association of 14 genotyped surrogates of rs7025486-A with AAA, both in Icelandic
(452 cases and 27,712 controls) and Dutch (840/2,791) samples sets and in the two sets combined,
and with MI (2,491/27,712), early-onset MI (697/27.712), PAD (1,477/27.712), VTE (946/27.712) and
PE (448/27.712) in Icelandic sample sets For the Icelandic sample set, information from additional
ungenotyped cases were included in the association analysis through family imputation. The Icelandic
results were adjusted for relatedness by dividing the chi2 statistic by 1.143, 1.411, 1.207, 1.422,
1.187 and 1.086, for AAA, MI, early-onset MI, PAD, VTE and PE, respectively.

| | | | | AAA | | | | | | MI | |
|---|---|---|---|---|---|---|---|---|---|---|---|
| | | | | Iceland | | Netherlands | | Comb. | | Iceland | |
| SNP | PosB36 | All | r2 | OR | P | OR | P | OR | P | OR | P |
| rs7873274 | 122599313 | C | 0.16 | 1.022 | 0.83 | 1.190 | 0.063 | 1.109 | 0.13 | 0.995 | 0.91 |
| rs2300939 | 122851538 | T | 0.12 | 1.060 | 0.62 | 1.245 | 0.039 | 1.158 | 0.062 | 1.094 | 0.11 |
| rs2777321 | 123396697 | T | 0.11 | 1.071 | 0.33 | 0.962 | 0.54 | 1.009 | 0.85 | 1.070 | 0.047 |
| rs2777319 | 123402323 | T | 0.22 | 0.967 | 0.59 | 1.048 | 0.43 | 1.009 | 0.83 | 0.962 | 0.19 |
| rs514823 | 123429574 | C | 0.11 | 1.063 | 0.45 | 0.969 | 0.67 | 1.011 | 0.84 | 1.017 | 0.65 |
| rs1003016 | 123443141 | C | 0.26 | 0.991 | 0.89 | 1.073 | 0.24 | 1.036 | 0.43 | 1.007 | 0.82 |
| rs12554667 | 123450426 | G | 0.30 | 1.044 | 0.51 | 1.010 | 0.87 | 1.025 | 0.57 | 1.018 | 0.57 |
| rs1571801 | 123467194 | G | 0.10 | 1.112 | 0.13 | 1.033 | 0.63 | 1.071 | 0.16 | 0.978 | 0.48 |
| rs10985354 | 123491526 | C | 0.12 | 0.974 | 0.67 | 1.053 | 0.38 | 1.015 | 0.73 | 1.009 | 0.76 |
| rs10818589 | 123549071 | T | 0.26 | 1.045 | 0.55 | 1.037 | 0.64 | 1.041 | 0.45 | 0.986 | 0.69 |
| rs12685856 | 123556411 | T | 0.11 | 1.169 | 0.22 | 0.994 | 0.97 | 1.088 | 0.38 | 0.947 | 0.37 |
| rs10818593 | 123600674 | T | 0.18 | 1.057 | 0.41 | 1.092 | 0.21 | 1.074 | 0.14 | 1.015 | 0.63 |
| rs12347373 | 123607488 | G | 0.12 | 0.938 | 0.31 | 1.079 | 0.2 | 1.011 | 0.8 | 1.020 | 0.51 |
| rs4641136 | 123611305 | G | 0.10 | 1.045 | 0.5 | 1.027 | 0.68 | 1.036 | 0.44 | 0.963 | 0.22 |

| | Early-onset | | PAD | | VTE | | PE | | Seq |
|---|---|---|---|---|---|---|---|---|---|
| | MI Iceland | | Iceland | | Iceland | | Iceland | | ID |
| SNP | OR | P | OR | P | OR | P | OR | P | NO: |
| rs7873274 | 0.978 | 0.8 | 1.045 | 0.5 | 0.985 | 0.83 | 0.913 | 0.33 | 56 |
| rs2300939 | 1.263 | 0.028 | 0.986 | 0.85 | 0.971 | 0.7 | 0.902 | 0.31 | 33 |
| rs2777321 | 1.080 | 0.21 | 1.080 | 0.099 | 1.010 | 0.84 | 1.024 | 0.73 | 40 |
| rs2777319 | 0.944 | 0.28 | 1.018 | 0.66 | 1.007 | 0.87 | 1.016 | 0.78 | 38 |
| rs514823 | 1.053 | 0.46 | 1.048 | 0.37 | 0.951 | 0.34 | 0.958 | 0.54 | 47 |
| rs1003016 | 1.095 | 0.093 | 1.004 | 0.93 | 1.016 | 0.72 | 1.048 | 0.42 | 1 |
| rs12554667 | 1.064 | 0.28 | 1.030 | 0.5 | 1.003 | 0.95 | 1.009 | 0.9 | 25 |
| rs1571801 | 0.943 | 0.33 | 0.960 | 0.37 | 0.988 | 0.8 | 0.961 | 0.53 | 27 |
| rs10985354 | 1.059 | 0.28 | 1.037 | 0.38 | 1.023 | 0.59 | 1.033 | 0.57 | 16 |
| rs10818589 | 0.986 | 0.82 | 0.955 | 0.34 | 0.957 | 0.38 | 0.969 | 0.63 | 11 |
| rs12685856 | 0.900 | 0.34 | 0.836 | 0.031 | 1.109 | 0.26 | 1.135 | 0.3 | 26 |
| rs10818593 | 1.073 | 0.23 | 0.976 | 0.58 | 0.985 | 0.74 | 1.028 | 0.64 | 12 |
| rs12347373 | 1.154 | 0.0094 | 1.013 | 0.76 | 1.041 | 0.36 | 0.994 | 0.91 | 20 |
| rs4641136 | 0.996 | 0.94 | 0.985 | 0.73 | 1.042 | 0.35 | 1.008 | 0.89 | 45 |

TABLE 11
(A) Association of surrogate markers of rs7025486 with the vascular diseases AAA, MI and early-onset MI for Icelandic cases and
controls, using imputed genotypes. R², frequencies and OR are presented for the predicted risk allele of the surrogate that is correlated to
allele A of rs7025486 based on publicly available Hap Map v24 and 1000 genome data. All P-values have been corrected for
genomic control.

| | | | | Ctrl | | | | | | | | | | |
|---|---|---|---|---|---|---|---|---|---|---|---|---|---|---|
| | | Risk | | (27,737) | AAA (421) | | | MI 2,563 | | | Early-onset MI (719) | | | Seq ID |
| SNP | Pos | All | R² | Frq | Frq | OR | P-val | Frq | OR | P-val | Frq | OR | P-val | NO: |
| rs878708 | 123385920 | A | 0.22 | 0.447 | 0.458 | 1.05 | 0.51 | 0.439 | 0.97 | 0.32 | 0.429 | 0.92 | 0.17 | 57 |
| rs669128 | 123392581 | C | 0.23 | 0.448 | 0.455 | 1.03 | 0.69 | 0.439 | 0.96 | 0.25 | 0.429 | 0.92 | 0.16 | 51 |
| rs2009828 | 123396379 | G | 0.22 | 0.432 | 0.435 | 1.01 | 0.89 | 0.423 | 0.96 | 0.23 | 0.415 | 0.93 | 0.22 | 32 |
| rs2777320 | 123397291 | A | 0.22 | 0.427 | 0.431 | 1.02 | 0.82 | 0.418 | 0.96 | 0.26 | 0.411 | 0.94 | 0.26 | 39 |
| rs4836858 | 123398134 | T | 0.22 | 0.431 | 0.434 | 1.01 | 0.87 | 0.422 | 0.96 | 0.24 | 0.414 | 0.93 | 0.23 | 46 |
| rs7038469 | 123400367 | T | 0.22 | 0.427 | 0.431 | 1.02 | 0.82 | 0.418 | 0.96 | 0.26 | 0.411 | 0.94 | 0.26 | 53 |
| rs2777319 | 123402323 | A | 0.22 | 0.427 | 0.434 | 1.03 | 0.73 | 0.418 | 0.96 | 0.25 | 0.410 | 0.93 | 0.24 | 38 |
| rs584985 | 123437111 | C | 0.2 | 0.309 | 0.314 | 1.02 | 0.78 | 0.317 | 1.04 | 0.28 | 0.347 | 1.21 | 0.0026 | 48 |
| rs2777311 | 123437209 | C | 0.25 | 0.341 | 0.341 | 1.00 | 0.97 | 0.346 | 1.03 | 0.41 | 0.370 | 1.17 | 0.018 | 37 |
| rs2777310 | 123439577 | T | 0.29 | 0.362 | 0.358 | 0.98 | 0.82 | 0.370 | 1.03 | 0.34 | 0.399 | 1.17 | 0.0072 | 36 |
| rs2797348 | 123439831 | G | 0.26 | 0.392 | 0.388 | 0.98 | 0.79 | 0.400 | 1.03 | 0.34 | 0.425 | 1.15 | 0.016 | 42 |
| rs12352132 | 123440666 | G | 0.36 | 0.339 | 0.339 | 1.00 | 0.98 | 0.345 | 1.03 | 0.38 | 0.374 | 1.19 | 0.0057 | 21 |
| rs62575880 | 123441329 | A | 0.38 | 0.372 | 0.370 | 0.99 | 0.89 | 0.380 | 1.03 | 0.32 | 0.409 | 1.18 | 0.0064 | 50 |
| rs2777308 | 123442163 | A | 0.31 | 0.369 | 0.366 | 0.99 | 0.84 | 0.376 | 1.03 | 0.33 | 0.409 | 1.19 | 0.0027 | 35 |
| rs2797347 | 123442393 | C | 0.25 | 0.403 | 0.397 | 0.98 | 0.75 | 0.409 | 1.03 | 0.44 | 0.433 | 1.13 | 0.031 | 41 |
| rs1003016 | 123443141 | G | 0.26 | 0.394 | 0.390 | 0.99 | 0.84 | 0.401 | 1.03 | 0.37 | 0.423 | 1.13 | 0.04 | 1 |
| s.123444035 | 123444035 | G | 0.32 | 0.371 | 0.370 | 1.00 | 0.97 | 0.380 | 1.04 | 0.25 | 0.410 | 1.19 | 0.0038 | 59 |
| s.123444070 | 123444070 | T | 0.53 | 0.258 | 0.245 | 0.93 | 0.39 | 0.265 | 1.05 | 0.27 | 0.296 | 1.26 | 0.00092 | 60 |
| rs1768732 | 123445098 | C | 0.37 | 0.371 | 0.370 | 1.00 | 0.98 | 0.379 | 1.04 | 0.26 | 0.410 | 1.19 | 0.0045 | 29 |
| s.123445547 | 123445547 | A | 0.32 | 0.371 | 0.370 | 1.00 | 0.98 | 0.379 | 1.04 | 0.27 | 0.410 | 1.19 | 0.0045 | 61 |
| s.123446322 | 123446322 | A | 0.2 | 0.118 | 0.125 | 1.10 | 0.47 | 0.117 | 0.99 | 0.85 | 0.130 | 1.16 | 0.14 | 62 |
| rs7465724 | 123446680 | C | 0.32 | 0.371 | 0.370 | 1.00 | 0.98 | 0.379 | 1.04 | 0.27 | 0.409 | 1.19 | 0.0045 | 54 |
| s.123446681 | 123446681 | A | 0.38 | 0.356 | 0.354 | 0.99 | 0.93 | 0.364 | 1.04 | 0.23 | 0.396 | 1.21 | 0.0024 | 63 |
| s.123447265 | 123447265 | A | 0.24 | 0.280 | 0.293 | 1.08 | 0.35 | 0.287 | 1.04 | 0.31 | 0.318 | 1.25 | 0.0012 | 64 |
| rs12553641 | 123447797 | A | 0.27 | 0.289 | 0.281 | 0.96 | 0.63 | 0.294 | 1.03 | 0.39 | 0.326 | 1.24 | 0.0013 | 23 |
| s.123449587 | 123449587 | T | 0.5 | 0.246 | 0.250 | 1.03 | 0.76 | 0.251 | 1.03 | 0.43 | 0.281 | 1.27 | 0.0012 | 65 |
| rs12554639 | 123450214 | A | 0.4 | 0.249 | 0.242 | 0.96 | 0.64 | 0.254 | 1.04 | 0.38 | 0.285 | 1.27 | 0.0011 | 24 |
| rs12554667 | 123450426 | G | 0.3 | 0.677 | 0.686 | 1.04 | 0.58 | 0.690 | 1.06 | 0.1 | 0.703 | 1.13 | 0.06 | 25 |
| s.123451318 | 123451318 | G | 0.76 | 0.273 | 0.291 | 1.11 | 0.23 | 0.278 | 1.03 | 0.51 | 0.313 | 1.25 | 0.00078 | 66 |
| s.123451324 | 123451324 | T | 0.25 | 0.125 | 0.128 | 1.04 | 0.73 | 0.125 | 1.00 | 0.97 | 0.131 | 1.07 | 0.48 | 67 |
| rs10818576 | 123452769 | G | 0.61 | 0.271 | 0.291 | 1.12 | 0.18 | 0.274 | 1.02 | 0.59 | 0.311 | 1.25 | 0.00081 | 4 |
| rs10760182 | 123452782 | G | 0.22 | 0.569 | 0.577 | 1.04 | 0.61 | 0.569 | 1.00 | 1 | 0.594 | 1.14 | 0.044 | 3 |
| rs10818577 | 123453012 | C | 0.89 | 0.273 | 0.289 | 1.09 | 0.29 | 0.276 | 1.02 | 0.63 | 0.312 | 1.23 | 0.0012 | 5 |
| rs10818578 | 123454572 | G | 1 | 0.305 | 0.337 | 1.16 | 0.056 | 0.315 | 1.05 | 0.18 | 0.350 | 1.22 | 0.00076 | 6 |
| rs10818579 | 123454726 | A | 0.96 | 0.294 | 0.324 | 1.16 | 0.065 | 0.301 | 1.03 | 0.35 | 0.337 | 1.23 | 0.00091 | 7 |
| rs10818580 | 123454843 | A | 1 | 0.298 | 0.327 | 1.14 | 0.086 | 0.305 | 1.03 | 0.38 | 0.340 | 1.21 | 0.0013 | 8 |
| s.123455512 | 123455512 | C | 0.88 | 0.294 | 0.324 | 1.16 | 0.065 | 0.301 | 1.03 | 0.35 | 0.337 | 1.23 | 0.00092 | 70 |
| rs10818582 | 123455826 | G | 0.3 | 0.626 | 0.642 | 1.09 | 0.3 | 0.633 | 1.04 | 0.37 | 0.655 | 1.17 | 0.019 | 9 |
| rs10985344 | 123456761 | A | 1 | 0.298 | 0.327 | 1.14 | 0.086 | 0.305 | 1.03 | 0.38 | 0.340 | 1.21 | 0.0013 | 13 |
| rs62572789 | 123458131 | A | 0.88 | 0.304 | 0.333 | 1.15 | 0.078 | 0.310 | 1.03 | 0.46 | 0.348 | 1.22 | 0.00087 | 49 |
| rs12380555 | 123458318 | C | 0.88 | 0.300 | 0.329 | 1.15 | 0.077 | 0.305 | 1.03 | 0.48 | 0.343 | 1.22 | 0.0011 | 22 |
| rs10985347 | 123459238 | T | 0.92 | 0.300 | 0.328 | 1.14 | 0.086 | 0.306 | 1.03 | 0.42 | 0.343 | 1.22 | 0.00098 | 14 |
| s.123459543 | 123459543 | C | 0.88 | 0.308 | 0.335 | 1.14 | 0.095 | 0.313 | 1.03 | 0.49 | 0.351 | 1.22 | 0.0011 | 71 |
| rs885150 | 123459994 | C | 1 | 0.308 | 0.339 | 1.15 | 0.065 | 0.317 | 1.04 | 0.23 | 0.353 | 1.22 | 0.00074 | 58 |
| s.123461786 | 123461786 | C | 0.85 | 0.315 | 0.346 | 1.15 | 0.064 | 0.321 | 1.03 | 0.45 | 0.357 | 1.21 | 0.0018 | 72 |
| rs10818583 | 123462082 | A | 1 | 0.298 | 0.327 | 1.14 | 0.086 | 0.305 | 1.03 | 0.38 | 0.340 | 1.21 | 0.0014 | 10 |
| s.123462115 | 123462115 | G | 0.2 | 0.695 | 0.711 | 1.10 | 0.27 | 0.696 | 1.01 | 0.83 | 0.707 | 1.08 | 0.28 | 73 |
| rs10985349 | 123465064 | T | 0.76 | 0.241 | 0.259 | 1.12 | 0.19 | 0.248 | 1.05 | 0.24 | 0.278 | 1.26 | 0.00098 | 15 |
| s.123469017 | 123469017 | C | 0.7 | 0.263 | 0.280 | 1.11 | 0.24 | 0.271 | 1.05 | 0.2 | 0.297 | 1.24 | 0.0021 | 74 |
| s.123472214 | 123472214 | A | 0.28 | 0.158 | 0.161 | 1.03 | 0.79 | 0.160 | 1.02 | 0.73 | 0.164 | 1.06 | 0.54 | 75 |
| s.123479977 | 123479977 | A | 0.25 | 0.150 | 0.159 | 1.11 | 0.39 | 0.154 | 1.05 | 0.37 | 0.163 | 1.17 | 0.1 | 76 |
| rs10818589 | 123549071 | T | 0.26 | 0.216 | 0.217 | 1.00 | 0.97 | 0.217 | 1.01 | 0.89 | 0.215 | 0.99 | 0.9 | 11 |
| rs10116069 | 123567781 | T | 0.26 | 0.213 | 0.213 | 1.00 | 1 | 0.215 | 1.01 | 0.84 | 0.211 | 0.98 | 0.81 | 2 |
| rs2416834 | 123591519 | C | 0.23 | 0.292 | 0.284 | 0.96 | 0.66 | 0.287 | 0.98 | 0.57 | 0.273 | 0.91 | 0.15 | 34 |

(B) Association of surrogate markers of rs7025486 with the vascular diseases PAD, VTE and PE for Icelandic cases and controls, using
imputed genotypes. R², frequencies and OR are presented for the predicted risk allele of the surrogate that is correlated to allele A
of rs7025486 based on publicly available Hap Map v24 and 1000 genome data. All P-values have been corrected for genomic control.

| | | | | Ctrl | | | | | | | | | | |
|---|---|---|---|---|---|---|---|---|---|---|---|---|---|---|
| | | Risk | | (27,737) | PAD (1,503) | | | VTE (1,013) | | | PE (478) | | | Seq ID |
| SNP | Pos | All | R² | Frq | Frq | OR | P-val | Frq | OR | P-val | Frq | OR | P-val | NO: |
| rs878708 | 123385920 | A | 0.22 | 0.447 | 0.449 | 1.01 | 0.82 | 0.448 | 1.00 | 0.96 | 0.451 | 1.02 | 0.81 | 57 |
| rs669128 | 123392581 | C | 0.23 | 0.448 | 0.450 | 1.01 | 0.82 | 0.448 | 1.00 | 1 | 0.453 | 1.02 | 0.77 | 51 |
| rs2009828 | 123396379 | G | 0.22 | 0.432 | 0.433 | 1.00 | 0.96 | 0.433 | 1.00 | 0.96 | 0.441 | 1.04 | 0.59 | 32 |
| rs2777320 | 123397291 | A | 0.22 | 0.427 | 0.427 | 1.00 | 0.99 | 0.429 | 1.01 | 0.87 | 0.435 | 1.03 | 0.63 | 39 |
| rs4836858 | 123398134 | T | 0.22 | 0.431 | 0.432 | 1.00 | 0.94 | 0.432 | 1.00 | 0.93 | 0.441 | 1.04 | 0.57 | 46 |
| rs7038469 | 123400367 | T | 0.22 | 0.427 | 0.427 | 1.00 | 0.99 | 0.429 | 1.01 | 0.87 | 0.435 | 1.03 | 0.63 | 53 |
| rs2777319 | 123402323 | A | 0.22 | 0.427 | 0.427 | 1.00 | 0.94 | 0.431 | 1.01 | 0.78 | 0.444 | 1.07 | 0.34 | 38 |
| rs584985 | 123437111 | C | 0.2 | 0.309 | 0.320 | 1.06 | 0.21 | 0.315 | 1.03 | 0.55 | 0.322 | 1.07 | 0.38 | 48 |
| rs2777311 | 123437209 | C | 0.25 | 0.341 | 0.353 | 1.07 | 0.14 | 0.349 | 1.05 | 0.41 | 0.358 | 1.10 | 0.23 | 37 |
| rs2777310 | 123439577 | T | 0.29 | 0.362 | 0.374 | 1.05 | 0.22 | 0.366 | 1.02 | 0.74 | 0.373 | 1.05 | 0.52 | 36 |
| rs2797348 | 123439831 | G | 0.26 | 0.392 | 0.399 | 1.03 | 0.51 | 0.402 | 1.04 | 0.39 | 0.412 | 1.09 | 0.24 | 42 |
| rs12352132 | 123440666 | G | 0.36 | 0.339 | 0.351 | 1.06 | 0.18 | 0.352 | 1.07 | 0.21 | 0.357 | 1.09 | 0.23 | 21 |
| rs62575880 | 123441329 | A | 0.38 | 0.372 | 0.381 | 1.04 | 0.35 | 0.382 | 1.05 | 0.39 | 0.390 | 1.08 | 0.29 | 50 |
| rs2777308 | 123442163 | A | 0.31 | 0.369 | 0.380 | 1.05 | 0.23 | 0.380 | 1.05 | 0.31 | 0.388 | 1.09 | 0.23 | 35 |
| rs2797347 | 123442393 | C | 0.25 | 0.403 | 0.408 | 1.03 | 0.55 | 0.411 | 1.04 | 0.47 | 0.418 | 1.07 | 0.33 | 41 |
| rs1003016 | 123443141 | G | 0.26 | 0.394 | 0.402 | 1.03 | 0.41 | 0.402 | 1.04 | 0.47 | 0.410 | 1.07 | 0.32 | 1 |
| s.123444035 | 123444035 | G | 0.32 | 0.371 | 0.381 | 1.05 | 0.3 | 0.382 | 1.05 | 0.34 | 0.390 | 1.09 | 0.24 | 59 |
| s.123444070 | 123444070 | T | 0.53 | 0.258 | 0.262 | 1.03 | 0.59 | 0.269 | 1.07 | 0.24 | 0.274 | 1.11 | 0.23 | 60 |
| rs1768732 | 123445098 | C | 0.37 | 0.371 | 0.381 | 1.05 | 0.3 | 0.382 | 1.05 | 0.34 | 0.390 | 1.09 | 0.24 | 29 |
| s.123445547 | 123445547 | A | 0.32 | 0.371 | 0.381 | 1.05 | 0.28 | 0.383 | 1.05 | 0.31 | 0.390 | 1.09 | 0.24 | 61 |
| s.123446322 | 123446322 | A | 0.2 | 0.118 | 0.130 | 1.17 | 0.031 | 0.130 | 1.17 | 0.075 | 0.129 | 1.15 | 0.24 | 62 |
| rs7465724 | 123446680 | C | 0.32 | 0.371 | 0.381 | 1.05 | 0.3 | 0.382 | 1.05 | 0.33 | 0.390 | 1.09 | 0.23 | 54 |
| s.123446681 | 123446681 | A | 0.38 | 0.356 | 0.366 | 1.05 | 0.24 | 0.370 | 1.07 | 0.19 | 0.379 | 1.12 | 0.14 | 63 |
| s.123447265 | 123447265 | A | 0.24 | 0.280 | 0.299 | 1.12 | 0.021 | 0.298 | 1.12 | 0.064 | 0.308 | 1.18 | 0.045 | 64 |
| rs12553641 | 123447797 | A | 0.27 | 0.289 | 0.297 | 1.05 | 0.3 | 0.305 | 1.10 | 0.087 | 0.312 | 1.14 | 0.091 | 23 |
| s.123449587 | 123449587 | T | 0.5 | 0.246 | 0.255 | 1.07 | 0.21 | 0.259 | 1.10 | 0.15 | 0.265 | 1.14 | 0.13 | 65 |
| rs12554639 | 123450214 | A | 0.4 | 0.249 | 0.256 | 1.05 | 0.38 | 0.262 | 1.10 | 0.15 | 0.269 | 1.15 | 0.12 | 24 |
| rs12554667 | 123450426 | G | 0.3 | 0.677 | 0.687 | 1.05 | 0.3 | 0.693 | 1.07 | 0.18 | 0.693 | 1.07 | 0.34 | 25 |
| s.123451318 | 123451318 | G | 0.76 | 0.273 | 0.296 | 1.14 | 0.0068 | 0.298 | 1.15 | 0.015 | 0.313 | 1.25 | 0.0052 | 66 |
| s.123451324 | 123451324 | T | 0.25 | 0.125 | 0.129 | 1.05 | 0.45 | 0.149 | 1.28 | 0.0013 | 0.152 | 1.32 | 0.0086 | 67 |
| rs10818576 | 123452769 | G | 0.61 | 0.271 | 0.293 | 1.13 | 0.0078 | 0.296 | 1.15 | 0.015 | 0.311 | 1.25 | 0.0055 | 4 |
| rs10760182 | 123452782 | G | 0.22 | 0.569 | 0.582 | 1.07 | 0.16 | 0.578 | 1.05 | 0.4 | 0.587 | 1.10 | 0.24 | 3 |
| rs10818577 | 123453012 | C | 0.89 | 0.273 | 0.294 | 1.12 | 0.013 | 0.301 | 1.16 | 0.0056 | 0.314 | 1.24 | 0.0037 | 5 |
| rs10818578 | 123454572 | G | 1 | 0.305 | 0.332 | 1.13 | 0.0032 | 0.333 | 1.14 | 0.014 | 0.344 | 1.20 | 0.014 | 6 |
| rs10818579 | 123454726 | A | 0.96 | 0.294 | 0.319 | 1.13 | 0.0058 | 0.321 | 1.14 | 0.015 | 0.333 | 1.21 | 0.011 | 7 |
| rs10818580 | 123454843 | A | 1 | 0.298 | 0.324 | 1.13 | 0.0052 | 0.328 | 1.15 | 0.0068 | 0.342 | 1.22 | 0.0042 | 8 |
| s.123455512 | 123455512 | C | 0.88 | 0.294 | 0.319 | 1.13 | 0.0058 | 0.321 | 1.14 | 0.015 | 0.333 | 1.21 | 0.011 | 70 |
| rs10818582 | 123455826 | G | 0.3 | 0.626 | 0.646 | 1.12 | 0.021 | 0.637 | 1.06 | 0.3 | 0.645 | 1.11 | 0.21 | 9 |
| rs10985344 | 123456761 | A | 1 | 0.298 | 0.324 | 1.13 | 0.0052 | 0.328 | 1.15 | 0.0066 | 0.342 | 1.22 | 0.0042 | 13 |
| rs62572789 | 123458131 | A | 0.88 | 0.304 | 0.330 | 1.13 | 0.0059 | 0.333 | 1.14 | 0.011 | 0.347 | 1.22 | 0.0073 | 49 |
| rs12380555 | 123458318 | C | 0.88 | 0.300 | 0.324 | 1.12 | 0.0093 | 0.329 | 1.15 | 0.011 | 0.342 | 1.22 | 0.0078 | 22 |
| rs10985347 | 123459238 | T | 0.92 | 0.300 | 0.325 | 1.13 | 0.0055 | 0.326 | 1.13 | 0.019 | 0.338 | 1.20 | 0.015 | 14 |
| s.123459543 | 123459543 | C | 0.88 | 0.308 | 0.332 | 1.12 | 0.0076 | 0.336 | 1.14 | 0.012 | 0.349 | 1.21 | 0.0095 | 71 |
| rs885150 | 123459994 | C | 1 | 0.308 | 0.335 | 1.13 | 0.0042 | 0.337 | 1.14 | 0.01 | 0.351 | 1.21 | 0.0073 | 58 |
| s.123461786 | 123461786 | C | 0.85 | 0.315 | 0.343 | 1.14 | 0.0032 | 0.343 | 1.14 | 0.015 | 0.356 | 1.21 | 0.01 | 72 |
| rs10818583 | 123462082 | A | 1 | 0.298 | 0.324 | 1.13 | 0.0053 | 0.328 | 1.15 | 0.0066 | 0.342 | 1.22 | 0.0042 | 10 |
| s.123462115 | 123462115 | G | 0.2 | 0.695 | 0.705 | 1.06 | 0.22 | 0.712 | 1.12 | 0.075 | 0.720 | 1.17 | 0.072 | 73 |
| rs10985349 | 123465064 | T | 0.76 | 0.241 | 0.260 | 1.13 | 0.013 | 0.266 | 1.17 | 0.0069 | 0.278 | 1.26 | 0.0045 | 15 |
| s.123469017 | 123469017 | C | 0.7 | 0.263 | 0.280 | 1.12 | 0.026 | 0.282 | 1.13 | 0.048 | 0.293 | 1.21 | 0.027 | 74 |
| s.123472214 | 123472214 | A | 0.28 | 0.158 | 0.160 | 1.02 | 0.72 | 0.175 | 1.17 | 0.03 | 0.173 | 1.16 | 0.16 | 75 |
| s.123479977 | 123479977 | A | 0.25 | 0.150 | 0.158 | 1.10 | 0.16 | 0.166 | 1.20 | 0.027 | 0.167 | 1.21 | 0.093 | 76 |
| rs10818589 | 123549071 | T | 0.26 | 0.216 | 0.220 | 1.02 | 0.66 | 0.217 | 1.01 | 0.93 | 0.209 | 0.96 | 0.61 | 11 |
| rs10116069 | 123567781 | T | 0.26 | 0.213 | 0.218 | 1.03 | 0.62 | 0.215 | 1.01 | 0.85 | 0.207 | 0.96 | 0.66 | 2 |
| rs2416834 | 123591519 | C | 0.23 | 0.292 | 0.292 | 1.00 | 0.97 | 0.297 | 1.03 | 0.61 | 0.294 | 1.01 | 0.89 | 34 |

1. A method of determining a susceptibility to a condition selected from the group consisting of abdominal aortic aneurysm, myocardial infarction, peripheral arterial disease and venous thromboembolism, the method comprising:
analyzing nucleic acid from a human individual for at least one polymorphic marker in the nucleic acid selected from the group consisting of rs7025486, and markers in linkage disequilibrium therewith, as characterized by values of r² of greater than 0.2;
wherein different alleles of the at least one polymorphic marker are associated with different susceptibilities to the condition in humans, and
determining a susceptibility to the condition for the human individual from the nucleic acid analysis.

2. The method of claim 1, wherein the nucleic acid is obtained from a biological sample containing nucleic acid from the human individual.

3. The method of claim 2, wherein the nucleic acid is analyzed to provide-sequence data using a method that comprises at least one procedure selected from:
(i) amplification of nucleic acid from the biological sample;
(ii) hybridization assay using a nucleic acid probe and nucleic acid from the biological sample; and
(iii) hybridization assay using a nucleic acid probe and nucleic acid obtained by amplification of the biological sample.

4-5. (canceled)

6. The method of claim 1, further comprising a step of preparing a report containing results from the susceptibility determination, wherein said report is written in a computer readable medium, printed on paper, or displayed on a visual display.

7. The method of claim 1, wherein the analyzing comprises determining the presence or absence of at least one at-risk allele of the polymorphic marker for the condition.

8. The method of claim 3, wherein the determining comprises comparing the sequence data to a database containing correlation data between the at least one polymorphic marker and susceptibility to the condition.

9. The method of claim 1, wherein markers in linkage disequilibrium with rs7025486 are selected from the group consisting of rs584985, rs2777310, rs2797348, rs12352132, rs2777308, rs2797347, rs1003016, rs12553641, rs12554667, rs10760182, rs10818577, rs10818578, rs10818579, rs10818580, rs10985344, rs885150, rs10818583, rs7025486, rs10985349, rs10818589, rs10116069, rs2416834, rs878708, rs669128, rs2009828, rs2777320, rs4836858, rs7038469, rs2777319, rs7869336, rs2797349, rs2777311, rs62575880, s.123444035, s.123444070, rs1768732, s.123445547, s.123446322, rs7465724, s.123446681, s.123447265, s.123449587, rs12554639, s.123451318, s.123451324, s.123451615, s.123451617, rs10818576, s.123455512, rs62572789, rs12380555, rs10985347, s.123459543, rs885150, s.123461786, s.123462115, rs1984038, rs1984037, s.123469017, s.123472214, rs12000685, rs12000723, rs35661033, s.123479977, rs1571804, s.123591409, and rs10985475.

10. The method of claim 7, wherein the at least one at-risk allele is selected from the group consisting of the rs7025486 allele A, rs584985 allele C, rs2777310 allele A, rs2797348 allele G, rs12352132 allele G, rs2777308 allele A, rs2797347 allele C, rs1003016 allele C, rs12553641 allele A, rs12554667 allele G, rs10760182 allele G, rs10818577 allele C, rs10818578 allele G, rs10818579 allele A, rs10818580 allele A, rs10985344 allele A, rs885150 allele C, rs10818583 allele A, rs7025486 allele A, rs10985349 allele T, rs10818589 allele T, rs10116069 allele T, rs2416834 allele C, rs878708 allele A, rs669128 allele C, rs2009828 allele G, rs2777320 allele A, rs4836858 allele T, rs7038469 allele T, rs2777319 allele T, rs7869336 allele T, rs2797349 allele A, rs2777311 allele C, rs62575880 allele A, s.123444035 allele G, s.123444070 allele T, rs1768732 allele C, s.123445547 allele A, s.123446322 allele A, rs7465724 allele C, s.123446681 allele A, s.123447265 allele A, s.123449587 allele T, rs12554639 allele A, s.123451318 allele G, s.123451324 allele T, s.123451615 allele T, s.123451617 allele A, rs10818576 allele G, s.123455512 allele C, rs62572789 allele A, rs12380555 allele C, rs10985347 allele T, s.123459543 allele C, rs885150 allele C, s.123461786 allele C, s.123462115 allele G, rs1984038 allele C, rs1984037 allele C, s.123469017 allele C, s.123472214 allele A, rs12000685 allele C, rs12000723 allele C, rs35661033 allele C, s.123479977 allele A, rs1571804 allele T, s.123591409 allele C, and rs10985475 allele T.

11. The method of claim 1, further comprising reporting the susceptibility to at least one entity selected from the group consisting of the individual, a guardian of the individual, a genetic service provider, a physician, a medical organization, and a medical insurer.

12. A method of determining a susceptibility of a human individual to a condition selected from the group consisting of abdominal aortic aneurysm, myocardial infarction, peripheral arterial disease and venous thromboembolism, the method comprising:
analyzing nucleic acid from the human individual for at least one polymorphic marker within the human DAB2IP gene;
wherein different alleles of the at least one polymorphic marker are associated with different susceptibilities to the condition in humans, and determining a susceptibility to the condition for the human individual from the nucleic acid analysis.

13. The method of claim 12, wherein the nucleic acid is obtained from a biological sample containing nucleic acid from the human individual.

14. The method of claim 13, wherein the nucleic acid is analyzed to provide sequence data using a method that comprises at least one procedure selected from:
(i) amplification of nucleic acid from the biological sample;
(ii) hybridization assay using a nucleic acid probe and nucleic acid from the biological sample; and
(iii) hybridization assay using a nucleic acid probe and nucleic acid obtained by amplification of the biological sample.

15-16. (canceled)

17. The method of claim 12, further comprising a step of preparing a report containing results from the susceptibility determination, wherein said report is written in a computer readable medium, printed on paper, or displayed on a visual display.

18. The method of claim 12, wherein the analyzing comprises determining the presence or absence of at least one at-risk allele of the polymorphic marker for the condition.

19. The method of claim 14, wherein the determining comprises comparing the sequence data to a database containing correlation data between the at least one polymorphic marker and susceptibility to the condition.

20. The method of claim 12, wherein the at least one polymorphic marker is selected from the group consisting of rs7025486, and markers in linkage disequilibrium therewith, as characterized by values of r² of greater than 0.2.

21-29. (canceled)

30. A computer-readable medium having computer executable instructions for determining susceptibility to a condition selected from the group consisting of abdominal aortic aneurysm, myocardial infarction, peripheral arterial disease and venous thromboembolism, the computer readable medium comprising:
a) data identifying the presence or absence of at least one allele of at least one polymorphic marker for at least one human subject;
b) a routine stored on the computer readable medium and adapted to be executed by a processor to determine risk of developing the condition for the at least one polymorphic marker;
wherein the at least one polymorphic marker is selected from the group consisting of rs7025486, and markers in linkage disequilibrium therewith, as characterized by values of r² of greater than 0.2.

31. The computer-readable medium of claim 30, wherein the medium contains data indicative for at least two polymorphic markers.

32. An apparatus for determining a genetic indicator for a condition selected from the group consisting of abdominal aortic aneurysm, myocardial infarction, peripheral arterial disease and venous thromboembolism, in a human individual, comprising:
a processor;
a computer readable memory;
wherein the computer readable memory has computer executable instructions adapted to be executed on the processor to analyze marker information for at least one human individual with respect to at least one polymorphic marker selected from the group consisting of rs7025486, and markers in linkage disequilibrium therewith, as characterized by values of r² of greater than 0.2, and generate an output based on the marker information, wherein the output comprises a measure of susceptibility of the at least one marker or haplotype as a genetic indicator of the condition for the human individual.

33. The apparatus of claim 32, wherein the marker information comprises sequence data identifying the presence or absence of at least one allele of the at least one marker in the genome of the individual.

34. The apparatus according to claim 33, wherein the computer readable memory further comprises data indicative of the risk of developing the condition associated with at least one allele of the at least one polymorphic marker, and wherein a risk measure for the human individual is determined based on a comparison of the marker information for the human individual to the risk of the condition associated with the at least one allele of the at least one polymorphic marker.

35. The method of claim 1, wherein the myocardial infarction is early onset myocardial infarction.

36. The method of claim 35, wherein the onset of myocardial infarction is before age 50 for men and before age 60 for women.

37. The method of claim 1, wherein the venous thromboembolism is pulmonary embolism.

38. The method according to claim 11, wherein the communication comprises making the determination of susceptibility available to the at least one person via a secure web site.

39. The method of claim 1, wherein the step of analyzing the nucleic acid sample comprises at least one nucleic acid analysis technique selected from: polymerase chain reaction, sequence analysis, analysis by restriction digestion, specific hybridization, single stranded conformation polymorphism assays (SSCP), electrophoretic analysis and expression analysis.

40. A method of using a nucleic acid sample isolated from a human individual to calculate a risk for developing breast cancer, the method comprising:
analyzing polymorphic marker rs7025486 in the nucleic acid sample, and
calculating a risk score for breast cancer for the individual that includes a genetic risk factor based on whether or not allele A of polymorphic marker rs7025486 is present in the sample.

41. The method of claim 40, wherein the step of analyzing the nucleic acid sample comprises at least one nucleic acid analysis technique selected from: polymerase chain reaction, sequence analysis, analysis by restriction digestion, specific hybridization, single stranded conformation polymorphism assays (SSCP), electrophoretic analysis and expression analysis.

42. The method of claim 41, further comprising reporting the susceptibility to at least one entity selected from the group consisting of the individual, a guardian of the individual, a genetic service provider, a physician, a medical organization, and a medical insurer.
